(19) United States
(12) Patent Application Publication
(10) Pub. No.: US 2014/0017301 A1
(43) Pub. Date: Jan. 16, 2014

(54) DRUG-FREE COMPOSITIONS AND METHODS FOR DIMINISHING PERIPHERAL INFLAMMATION AND PAIN

(71) Applicant: Gregor Cevc, Gauting (DE)

(72) Inventor: Gregor Cevc, Gauting (DE)

(21) Appl. No.: 14/006,948

(22) PCT Filed: Mar. 21, 2012
(86) PCT No.: PCT/EP2012/055039

§ 371 Date: Sep. 23, 2013

Related U.S. Application Data
(60) Provisional application No. 61/454,734, filed on Mar. 21, 2011.

(51) Int. Cl. A61K 9/127

(52) U.S. Cl. CPC A61K 9/1272
USPC 424/450; 514/552; 514/723; 514/718; 514/473; 514/54; 514/23; 514/53; 514/148; 514/517

(57) ABSTRACT

The present invention provides drug-free adaptable aggregate compositions, typically having a form of bilayer vesicles suspended in a polar, optionally thickened, fluid comprising different pharmaceutically acceptable excipients for use in or on a mammal for any medical indication, specifically for non-invasive treatment of local inflammation and the associated pain, in particular for use on the skin and underlying tissues, including muscles and/or superficial joints. Accompanying guidelines for selecting components to thereby optimizing the formulations are also provided.

### 1. FIELD OF THE INVENTION

This invention relates to the use of multi-component formulations useful for the non-invasive treatment of local inflammation and associated pain, in particular for use on the skin and underlying tissues, including muscles and/or superficial joints.

### 2. BACKGROUND OF THE INVENTION

Treatment of local inflammation and associated pain remains a medical challenge. To ameliorate these conditions, patients typically use pharmacological anti-inflammatory agents, frequently from the class of non-steroidal anti-inflammatory drugs (NSAIDs), despite the common resulting adverse side effects. To date, no practically attractive and satisfactory solution to this problem is known. In particular, there is no known drug free preparation that can be applied locally to treat localized inflammation and pain that provides meaningful clinical benefits comparable to known compositions, including NSAID preparations. Furthermore, there are no drug-free formulations proven to be more clinically effective in alleviating pain and inflammation than known locally applied negative controls that would present a favourable alternative to known pharmacological options.

One potent NSAID available in a semi-solid form (diclofenac in Voltaren® Emulgel, Novartis) has been experimentally shown to be superior to a drug-free negative control preparation, but only when this NSAID was used frequently and abundantly. Less frequent application of a different NSAID (ketoprofen) has also produced clinical benefits on par with an oral selective NSAID (a celecoxib), but only when the drug was associated with ultradeformable vesicles based on soybean phosphatidylcholine. The same study confirmed advantages of the vesicular NSAID product over the corresponding drug-free vesicles. In a different study, ketoprofen-loaded vesicles gave inconclusive results when compared with another oral NSAID (naproxen) or the corresponding drug-free vesicles.

Local anti-inflammatory, or antiphlogistic, activity of phosphatidylcholine was described some time ago, in particular the beneficial effects of a topically applied liposome formulation against dermatitis, composed of phosphatidylcholine with 60% linoleic (i.e. polyenyl) chains. The therapeutic activity of polyenylphosphatidylcholine (“PPC”) formulated in a topical composition comprising about 0.1% to about 10% by weight PPC, preferably from soybean, in a dermatologically acceptable carrier has also been discussed. Other studies have generally examined anti-inflammatory effects of certain phospholipids including one study relating to vesicular formulations having one or more phospholipids (including lysolipids) and one or more nonionic surfactants for treating deep tissue pain, e.g. osteoarthritis and other joint or muscle pain. But, these compositions required a surfactant/phospholipid weight ratio of 1/1 to 1/5 w/w.

What is needed are improved compositions and related methods of use for diminishing and/or treating peripheral pain and/or inflammation that imparts minimal side effects to a subject and is optimally easy to use. Such compositions should ideally afford drug-free alternatives, including preparations with fewer or no phospholipids compared to existing preparations, while providing stability and/or other commercial advantages. The compositions should moreover ideally comprise a variety of chemical substances for improved treatment versatility.

### 3. SUMMARY OF THE INVENTION

The present invention discloses various amphipat combinations, preferably in form of bilayer vesicles, which effectively suppress inflammation and commonly associated pain. Such combinations are drug-free but can nonetheless favourably affect symptoms associated with local inflammation, including (osteo)arthritis, when the amphipats are applied locally in the form of sufficiently adaptable bilayer vesicle aggregates. As explained herein, the effectiveness of these combinations surprisingly appears to relate to physical and structural considerations rather than to chemical characteristics of the disclosed vesicular aggregates. Moreover, most of the vesicles and associated beneficial effects do not require phosphatidylcholine/phospholipid and/or may optionally include a phospholipid component, but not in certain concentration ranges previously known in the art.

A further goal of the invention is the identification of compositions that yield aggregates in the form of deformable, adaptable bilayer vesicles such that they can physically interact with the skin and underlying tissues to ameliorate undesirable conditions, in particular inflammation and associated pain. To meet the goal, the invention provides three selection criteria useful for establishing the desired vesicle formulations.

The first criterion identifies and links certain limiting average area per hydrophobic chain aspects with sufficient bilayer deformability of the vesicular compositions. The second criterion identifies certain ranges of amphipat headgroup polarities that ensure the corresponding amphipat bilayer deformability to be high, and therefore sufficient for the desired adaptable vesicle interaction with the skin and/or penetration through the cutaneous barrier. The third criterion defines certain ranges of Hydrophilicity-Lipophilicity-Balance (HLB) numbers corresponding to amphipat mixtures that beneficially afford highly adaptable (vesicular) aggregates with the desired anti-inflammatory activity. Any of these criteria can be used independently to select suitable amphipats and their relative concentrations with sufficient precision for purposes of the invention, considering the underlying information about molecular structure and/or amphipat packing in the described formulations. However, while all of the criteria are useful, the first criterion appears to be the most accurate and the third criterion is the least accurate amongst the three, when applied to amphipats having a similar chain length. This invention thus effectively eliminates the previous, and often tedious, requirement of identifying therapeutically useful formulations via extensive, often trial and error, experimentation.

An additional aim of the present invention is to widen the spectrum of adaptable vesicles with a highly deformable bilayer, useful for localized application and treatment of peripheral inflammation and pain, beyond the known vesicle preparations that are based on phosphatidylcholine components. The invention thus provides a ready means to identify particularly effective amphipat combinations from the many potentially suitable choices, by describing assessment techniques supporting the prediction and/or confirmation of the beneficial effect of said combinations on a local inflammation and pain. These techniques are convenient, relatively inexpensive, and suitable for an easy comparison of the new with the known formulations used for local inflammation and pain treatment, the latter requiring either drug components and/or narrowly defined phospholipid components.

A further aim of the invention is to provide various therapeutically beneficial amphipat combinations for vesicles development, including combinations of single- and multi-chain amphipats, of amphipats with different headgroups (polyoxyethylene, polysorbate, polyglyceride) and of amphipats with 1, 2, or 3 double-bonds in the aliphatic chain. The presented biological evidence and analyses imply that anti-inflammatory effects of locally applied adaptable aggregates do not rely on any particular molecular species. In contrast, the same evidence suggests that relatively rigid drug-free vesicles (such as empty liposomes) are inactive, whereas highly adaptable vesicles can, surprisingly, be nearly as biologically active as drug-loaded aggregates or commercial topical NSAID preparations.

The invention further discloses suitable manufacturing processes, dosages, and suggested schedules for the described formulations applications that ensure a consistent and sufficient therapeutic effect. The invention thus offers unprecedented drug- and side-effect-free options widely suitable for treating mammalian subjects, in particular humans.

### 4. BRIEF DESCRIPTION OF THE DRAWINGS

FIG. 1 is a graphical representation of experiments carried out to show a suppression of mustard oil induced human skin erythema by amphipat mixtures differing in chain unsaturation (C18:1, C18:2, C18:3), headgroups (partially ionic (C18:1, C18:2, C18:3), zwitterionic (C18:2/C18:2 PC), non-ionic (S80/T80, T81/80, T85/80, EmOG/T80)) which all share an ability to form adaptable vesicles in aqueous media (grey columns). Topical NSAIDs (Voltaren® Emulgel® (Novartis) and Ketoprofen gel were employed as positive controls (shown as black columns). The simpler lipid vesicles (a drug-free Liposome gel) and commercial hydrocortisone solution were used as negative controls (shown as white columns). The short horizontal lines identify the mean values of the different independently conducted tests. The vertical bar illustrates the estimated intra-experiment standard deviation. The dashed horizontal line identifies 100% treatment success and the dotted horizontal lines indicate the negative control variability.

### 5. DETAILED DESCRIPTION OF THE INVENTION

Unless defined otherwise, all technical and scientific terms used herein have their plain, general meaning understandable to one of ordinary skill in the art in the relevant technical field.

The term “about”, or “around” when used with a numerical value, means a range surrounding the corresponding numerical value, including the typical measuring error associated with a particular experiment. Unless specifically stated to be, e.g., ±1%, ±2%, ±3%, ±4%, ±5%, ±10%, ±15%, ±20%, ±25%, ±30%, ±35%, ±40% or any other percentage of the numerical value, the term “about” or “around” used in connection with a particular numerical value generally means±25%. For imprecisely known or not uniquely defined quantities, this term implies a range of ±50%.

The term “acyl” means a linear hydrocarbon radical with 2 to 30 (C₂₋₃₀), 2 to 24 (C₂₋₂₄), 2 to 22 (C₂₋₂₂), 2 to 20 (C₂₋₂₀), 2 to 18 (C₂₋₁₈), 2 to 16 (C₂₋₁₆), 2 to 14 (C₂₋₁₄), 2 to 12 (C₂₋₁₂), or 2 to 10 (C₂₋₁₀) C-atoms. Standard nomenclature is as follows: a chain with C30:0=triacontyl, C24:0=tetracosanoyl (or lignoceryl), C23:0=tricosanoyl, C22:0=docosanoyl (or behenyl), C21:0=heneicosanoyl, C20:0=eicosanoyl (or arachidoyl), C19:0=nonadecanoyl, C18:0=octadecanoyl (or stearoyl), C17:0=heptadecanoyl (or margaroyl), C16:0=hexadecanoyl (or palmitoyl), C15:0=pentadecanoyl, C14:0=tetradecanoyl (or myristoyl), C13:0=tridecanoyl, C12:0=dodecanoyl (or lauroyl), C10:0=decanoyl (or capryl), C8:0=octanoyl (or capryloyl), C7:0=heptanoyl, C6:0=hexanoyl (or caproyl), C5:0=pentanoyl, C4:0=butanoyl/butyric), C3:0=propanoyl (or propionyl), C2:0=acetyl.

The term aggregate “adaptability” is herein practically synonymous with bilayer “deformability” and can be measured with previously described methods (e.g. Wachter et al., 2008, J. Drug Targeting 16: 611). In principle, these methods assess the penetration of a nanoporous, semipermeable barrier by the tested aggregates in a suspension, presuming no significant aggregate fragmentation. In an alternative method, the kinetics of aggregate fragmentation under external stress is studied, e.g., during ultrasonication. An aggregate is considered to be ultra-adaptable (or ultradeformable) for purposes of the invention if its adaptability is close to the highest value achievable without an appreciable, and normally spontaneous, aggregate fragmentation into smaller structures, e.g. micelles. An alternative criterion useful for the purpose is achieving at least 5-times, more preferably 10-times, or even more preferably, 20-times shorter enforced vesicularisation time compared with more conventional lipid bilayer vesicles (e.g. with the reference fluid-phase liposomes made of >95% pure phosphatidylcholine) under comparable conditions. Confirmation of functional similarity between any newly tested formulation and a formulation previously shown to yield ultradeformable vesicles can prove the point as well.

To confirm aggregate stability, one can check the average aggregate size before and after pore crossing, or before and after another kind of external stress application. Photon correlation spectroscopy (PCS or dynamic light scattering) or turbidity spectrum analysis (e.g. in Elsayed & Cevc, 2011, Pharm. Res. 28: 2204) can be used for such purpose. The simplest option is comparing opalescence of the test preparation with opalescence of a suitable reference suspension containing stable small aggregates to detect differences, if any, after correction for light absorption, if any. To confirm the existence of a bilayer, if necessary, one can use an osmotic swelling-test, X-ray, or neutron scattering, for example, or any other method known in the art to reveal presence of an inner aggregate volume and its segregation from the outer volume (such as vesicle leakage). To test preparation stability under more physiological conditions, one can measure the skin surface conductivity and/or hydration as a function of time after the test formulation application on the skin surface: appreciable residual superficial water content, measured after a practically relevant drying time (e.g. 10 min), indicates aggregate stability sufficient for purposes of the invention. Label content or application-dependent activity in—or even beyond—the treated skin may also demonstrate the tested formulation functionality.

The term “aliphatic” chain herein means a non-aromatic straight or branched hydrocarbon chain joined together by single bonds (alkanes) and/or double bonds (alkenes), and/or less preferably triple bonds (alkynes). Examples include straight or branched alkenyl, alkyl, and alkynyl chains with 1, 2, 3, 4, 5, or 6 double and/or 1, 2, or 3 triple bonds and/or alkoxy or polyoxy-alkylenes with 1, 2, 3, 4, 5, 6, or 7 hydroxy side groups. Each such chain may moreover have 0, 1, 2, 3, 4, 5, or 6 side branches. An aliphatic chain can moreover contain one or more non-aromatic rings, as in cycloalkanes and heterocyclyl residues. Many aliphatic chains may be derived from oils, e.g. by alkaline hydrolysis. For purposes of this invention, the term aliphatic also includes suitable fluorohydrocarbon analogues to any of the amphipathic compounds described herein.

The term “alkanoyl” is a synonym for “acyl”.

The term “alkenoyl” means a —C(O)-alkenyl.

The term “alkenyl” means a linear or branched monovalent hydrocarbon radical containing one or several carbon-carbon double bonds in either (the more preferred) “cis” or (the less preferred) “trans” configuration, which can also be written as “Z” or else “E”, respectively. (Such preference for the cis-configuration is not generally transferable to other kind of molecules, which can thus be used in either of the two configurations, unless stated otherwise.) The radical can be substituted with one or several chemically suitable substituents. The alkenyl is typically a linear monovalent hydrocarbon radical with 2 to 30 (C₂₋₃₀), 2 to 24 (C₂₋₂₄), 2 to 22 (C₂₋₂₂), 2 to 20 (C₂₋₂₀), 2 to 18 (C₂₋₁₈), 2 to 16 (C₂₋₁₆), 2 to 14 (C₂₋₁₄), 2 to 12 (C₂₋₁₂), 2 to 10 (C₂₋₁₀), or 2 to 8 (C₂₋₈) C-atoms. When branched, the alkenyl typically contains 3 to 30 (C₃₋₃₀), 3 to 24 (C₃₋₂₄), 3 to 22 (C₃₋₂₂)_{,} 3 to 20 (C₃₋₂₀), 3 to 18 (C₃₋₁₈), 3 to 16 (C₃₋₁₆), 3 to 14 (C₃₋₁₄), 3 to 12 (C₃₋₁₂), 3 to 10 (C₃₋₁₀), or 3 to 8 (C₃₋₈) carbon (C) atoms. The shorter alkenyl chains with 3 to 6 (C₃₋₆) C-atoms are not particularly attractive for purposes of the invention as a lipid component, but may be valuable as part(s) of an organic ion. The preferred short-chain alkenyl radicals especially useful as parts of organic ions incorporated into a preparation include, but are not limited to allyl, butenyl, ethenyl, 4-methylbutenyl, propen-1-yl, and propen-2-yl radicals.

A mono-alkenyl or alkenoyl contains one carbon-carbon double bond. If not specified as a mono-alkenyl or -alkenoyl, an alkenyl- or alkenoyl can be a dialkenyl or -alkenoyl, and then contain two carbon-carbon double bonds, or an oligo- or poly-alkenyl or -alkenoyl (i.e. polyenyl), and then contain more than two, and preferably 3 or 4, carbon-carbon double bonds. Mono-alkenoyls with longer chains include, but are not limited to 15c-24:1=C24:1(n-9) or nervonic, 13c-22:1=C22:1(n-9) or erucic, 11c-20:1=C20:1(n-9) or gondoic, 6c-18:1=C18:1(n-12) or petroselinic, 9c-18:1=C18:1(n-9) or oleic, 11c-18:1=C18:1(n-7) or cis-vaccenic, or the less preferred 9t-18:1 or elaidic and 11t-18:1 or vaccenic, furthermore 7c-16:1=C16:1(n-9)=cis-7-hexadecenoic, 9c-16:1=C16:1(n-7) or palmitoleic, or the less preferred 3t-18:1=trans-3-hexadecenoic, and finally 9c-14:1=C14:1(n-5) or myristoleic radical. The oligo-alkenoyl radicals of C22-class of note include 13c,16c-22:2=C22:2(n-6)=13,16-docosadienoic, 13c,16c,19c-22:3=C22:3(n-3)=13,16,19-docosatrienoic, 10c,13c,16c-22:3=C22:3(n-6)=10,13,16-docosatrienoic, 7c,10c,13c,16c-22:4=C22:4(n-6)=7,10,13,16-docosatetraenoic (or adrenic), 4c,7c,10c,13c,16c,19c-22:5=C22:6(n-3)=4,7,10,13,16,19-docosahexaenoic, 4c,7c,10c,13c,16c-22:5=C22:5(n-6)=4,7,10,13,16-docosapentaenoic acid. The main oligo-alkenoyls with 20-C-atoms are 14c,17c-20:2=C20:2(n-3)=14-cis,17-cis-eicosadienoic, 11c,14c-20:2=C20:2(n-6)=11-cis,14-cis-eicosadienoic, 11c,14c,17c-20:3=C20:3(n-3) or dihomo-α-linolenic, 8c,11c,14c-20:3=C20:3(n-6) or dihomo-gamma-linolenic, 5c,8c,11c-20:3=20:3(n-9) or ‘Mead's’, 5c,8c,11c,14c-20:4=C20:4(n-6) or arachidonic, 8c,11c,14c,17c-20:4=C20:4(n-3)=8,11,14,17-all-cis-eicosatetraenoic, and 5c,8c,11c,14c,17c-20:5=C20:5(n-3)=5,8,11,14,17-all-cis-eicosapentaenoic acid. Interesting C18 oligo- and poly-alkenoyls, include but are not limited to 12c,15c-18:2=C18:2(n-3) or alpha-linoleic, 10c,12t-18:2=C18:2(n-6)=trans-10,trans-12-octadecadienoic, 9c,12c-18:2=C18:2(n-6) or gamma-linoleic, 9c,12c,15c-18:3=C18:3(n-3) or alpha-linolenic, 6c,9c,12c-18:3=C18:3(n-6) or gamma-linolenic, 9c,11c,13t-18:3 or alpha-eleostearic, 8t,10t,12c-18:3 calendic, 6c,9c,12c,15c-18:4=C18:4(n-3) or stearidonic, 3c,6c,9c,12c-18:4=C18:4(n-6)=3,6,9,12-octadecatetraenoic, 3c,6c,9c,12c,15c-18:5=C18:5(n-3)=3,6,9,12,15-octadecapentaenoic acid. The main oligo-/poly-alkenoyls with C16 are 10c,13c-16:2=C16:2(n-3)=10-cis,13-cis-hexadecadienoic, 7c,10c-16:2=C16:3(n-6)=7-cis,10-cis-hexadecadienoic, 7c,10c,13c-16:3=C16:3(n-3)=7-cis,10-cis,13-cis-hexadecatrienoic acid. The above listing is not exhaustive, as other double bond combinations are possible and useful for the invention. Chains having more than three double bonds per chain, however, are less preferred than mono-, di- and tri-unsaturated chains. Any number of double bonds per chain that is smaller than the maximum possible number indicates “partial saturation”, but the preferential meaning of this term is 1, 2, or three double bonds per chain, preferably in the cis-configuration.

The term “alkyl” refers to a linear or branched saturated monovalent hydrocarbon radical that can include one or several substituents. The alkyl is typically a linear saturated monovalent hydrocarbon radical with 1 to 30 (C₁₋₃₀ or C1-C30:0), 1 to 24 (C₁₋₂₄ or C1-C24:0), 1 to 22 (C₁₋₂₂ or C1-C22:0), 1 to 20 (C₁₋₂₀ or C1-C20:0), 1 to 18 (C₁₋₁₈ or C1-C18:0), 1 to 16 (C₁₋₁₆ or C1-C16:0), 1 to 14 (C₁₋₁₄ or C1-C14:0), 1 to 12 (C₁₋₁₂ or C1-C12:0), 1 to 10 (C₁₋₁₀ or C1-C10:0), 1 to 6 (C₁₋₆ or C1-C6:0), 1 to 4 (C₁₋₄ or C₁-C4:0), or 1 to 2 (C₁₋₂ or C1-C2) C-atoms (the “0” in Cx:0 denoting absence of carbon-carbon double bonds); if branched, alkyl is a saturated monovalent hydrocarbon radical with 3 to 30 (C₃₋₃₀), 3 to 24 (C₃₋₂₄), 3 to 22 (C₃₋₂₂), 3 to 20 (C₃₋₂₀), 3 to 18 (C₃₋₁₈), 3 to 14 (C₃₋₁₄), 3 to 12 (C₁₃₋₁₂), 3 to 10 (C₃₋₁₀), or 3 to 6 (C₃₋₆), C-atoms. The commonly used names for some types of alkyls include: C30:0=triacontanoic, C24:0=lignoceric, C23:0=tricosanoic, C22:0=behenic, C21:0=heneicosanoic, C20:0=arachidic, C19:0=nonadecanoic, C18:0=stearic, C17:0=margaric, C16:0=palmitic, C15:0=pentadecanoic, C14:0=myristic, C13:0=tridecanoic, C12:0=lauric, C10:0=capric, C8:0=caprylic, C7:0=heptanoic, C6:0=caproic, C5:0=valeric, C4:0=butyric, C3:0=propionic, C2:0=acetic. Monobranched (e.g. iso-stearic, iso-palmitic, iso-myristic or even iso-lauric) or multi-branched (e.g. Guerbet alcohols such as butyloctanol with 12 C-atoms, hexyldecanol with 16 C-atoms, octyldodecanol with 20 C-atoms and decyldodecanol with 22 C-atoms) aliphatic chains may be useful in the aggregates of the invention due to their high oxidation stability and low melting point. Chain melting temperatures of all suitable fatty residues are published or can be readily derived.

The linear C₁₋₆ and branched C₃₋₆ alkyl groups are in the context of this invention sometimes described as “lower alkyls.” Examples of alkyl groups include, but are not limited to, methyl, ethyl, propyl (and all its isomers, such as n-propyl, isopropyl), butyl (and all its isomers, such as n-butyl, isobutyl, sec-butyl, t-butyl), pentyl (and all its isomers, such as n-pentyl, iso-pentyl, sec-pentyl, t-pentyl, q-pentyl), and hexyl (and all its isomers) or heptyl (and all its isomers). Lower alkyls play only a limited, if any, role as parts of lipids forming the aggregates of the invention. Lower alkyls can be attractive as parts of the described additives, however, as aggregate-modifying anions or cations, C1 to C8, more preferably C2 to C7 and most preferably C2 to C6 are used.

The term “amphipat” or “amphiphile” (adjective: “amphipathic”) refers to a chemical compound possessing both hydrophilic and lipophilic properties, i.e. an amphipathic molecule. The words “amphipat” and “lipid” are used herein interchangeably.

The terms “anion” and “anionic group” means herein any negatively charged atom or group of atoms, typically soluble in water and having a tendency to migrate to an anode in an electrolytic cell, including combinations and/or substituted forms thereof.

The term “antimicrobial” agent, or microbicide, means at least one, and more frequently a combination of, substance(s) that reduce pathogen count and/or prevent pathogen growth in the preparations if included; pathogens in this context are mainly bacteria, yeast, fungi and mold, plus potentially viruses. Potentially useful microbicides include but are not limited to certain simple acids (such as formic, acetic, propionic, sorbic, lactic, naphtenic or salicylic acid, etc.), their pharmacologically acceptable halogenated derivatives, such as bromoformic, bromoacetic or trifluoroacetic acid, as well as their alkyl, esp. lower-alkyl, such as ethyl- or else benzyl-derivatives, such as alkyl-benzoic acids, but also dehydroacetic acid, edetic acid (EDTA), Br-benzyl-teta; acid releasing substances such as dimethoxane, short-chain (i.e. lower-alkyl, etc.) mono-, di-, and triols (such as ethanol, propanol, propanediol, butanediol, pentanediol, ethylhexyl glycerol, caproyl glycol, etc.), acrolein (i.e. 2-propenal); aryl substituted alcohols, such as 2- or 1-phenylethanol, phenoxyethanol or phenoxyisopropanol, menthol; or aryl- and hetero-aryl-substituted halides, octylisothiazolinone, chlorbenzyl alcohol, chlorbutanol, chlorhexidine, chloroxylenol, dichlorbenzylalcohol, dichlorophene, iodopropynyl butylcarbamate (IPBC); acrolein (2-propenal); N-(hydroxylmethyl)glycine or its salt; biocides acting via bromonitromethane donation (including but not limited to the commercially available 2-bromo-2-nitroethenyl furan, 2-bromo-2-nitropropane-1,3-diol (Bronopol), 5-brom-5-nitro-1,3-dioxanand (beta-bromo-beta-nitro)styrene, 2-bromo-2-bromo-methylpentane-dinitrile, 2-bromo-2-(bromomethyl)pentanedinitrile, methyldi-bromo glutaronitrile, 2-(2-bromo-2-nitroethenyl)furan), silver chloride on TiO₂, diiodo-methyl-p-tolylsulfone, iodopropynyl butylcarbamate, etc.), benzisothiazolone, bisabolol, dimethoxydimethyl hydantoin, various dibenzamidines, members of the halogen alkenyl azolyl class, hexylresorcinol, methylbenzethonium chloride, benzyl alcohol, eucalyptol, glutaraldehyde, hexachlorophene, menthol, diazolidinyl urea and imidazolidinyl urea or tetra methylol acetylene diurea, parabenes, phenolic compounds, phenoxyethanol, povidon-iodine, phytantriol; sulfanimide, such as 4-aminobenzenesulfonamide, quaternary ammonium compounds, halogen alkenyl azolyl microbicides; triclosan (e.g. irgasan); methylchloroisothiazolinone (MCIT) or methylisothiazolinone (MIT); mercurial compounds; thymol; alkyl-salicylamides, esp. with C4-C14 chains, salicylanilide, intermediate-chain (such as lauryl-) surfactants or -alcohols with a proven anti-microbial activity; quaternium-15; antibiotic peptides, or any other pharmacologically acceptable substance of biological origin, or a suitable mixture thereof. Additional potentially useful antimicrobial compounds are listed e.g. in “Directory of Microbicides for the Protection of Materials. A Handbook (in two parts, W. Paulus, ed.), Springer, Berlin, 2005.

The term “antioxidant” means any substance suppressing oxidation in the formulations, including but not limited to aromatic amines (e.g. diphenylamine), ascorbic, kojic and malic acid and their salts (isoascorbate, (2 or 3 or 6)-o-alkylascorbic acids, or esters, especially of the alkyl- and alkenoyl-type, alkylated, e.g. butylated, hydroxylanisol (BHA) or hydroxytoluene (BHT), moreover, 3,5-di-tertbutyl-4-hydroxybenzyl alcohol and 2,6-ditert-butylphenol; tert-butylhydroquinone (TBHQ), trimethylhydroquinone and its alkyl-derivatives, such as 1-O-hexyl-2,3,5-trimethylquinol (HTHQ); carbazol, ellagic acid, ethylenediamine derivatives, eugenol, gallic acid or one of its esters (e.g. an alkyl-, such as ethyl-, propyl- or butyl-gallate), thioglycerol, nordihydroguaiaretic acid (NDGA), p-alkylthio-o-anisidine, a phenol or a phenolic acid; tetrahydroindenoindol; thymol; tocopherol and its derivatives (lipoates, succinates and —POE-succinates); trolox and the corresponding amide and thiocarboxamide analogues; quinic acid, vanillin. Also useful are the preferentially oxidizable compounds, such as sodium bisulphite, sodium metabisulphite, thiourea, as well as chelating agents, such as EDTA, EGTA, -bis-N,N′-tetraacetic acid, triglycine, EDDS, BAPTA, desferoxamine, etc., any of which may be suitably used as a secondary “antioxidant”. Further useful antioxidants include endogenous defense systems, such as cearuloplasmin, heamopexin, ferritin, haptoglobion, lactoferrin, transferrin, ubiquinol-10), enzymatic antioxidants; the less complex molecules including but not limited to N-acetylcysteine, bilirubin, caffeic acid and its esters, beta-carotene, cinnamates, flavonoids, glutathione, mesna, tannins, thiohistidine derivatives, triazoles, uric acid; spice extracts; carnosic acid, carnosol, carsolic acid; rosmarinic acid, rosmaridiphenol; oat flour extracts, gentisic acid and phytic acid, steroid derivatives (e.g., U74006F); tryptophan metabolites, and organochalcogenides.

The term “area per chain”, or Ac, means herein the average molecular area divided by the number of hydrophobic (most often aliphatic) chains per molecule. Experimental Ac values are typically method and readout dependent, and should therefore be compared on ‘like-with-like’ basis.

The term “aryl” means a monocyclic aromatic group and/or a multicyclic monovalent aromatic group containing at least one aromatic hydrocarbon ring. The aryl will thus typically contain from 6 to 30 (C₆₋₃₀), from 6 to 24 (C₆₋₂₄), from 6 to 22 (C₆₋₂₂), from 6 to 20 (C₆₋₂₀), from 6 to 18 (C₆₋₁₈), from 6 to 16 (C₆₋₁₆), from 6 to 14 (C₆₋₁₄), from 6 to 12 (C₆₋₁₂), or from 6 to 10 (C₆₋₁₀) atoms. This includes, but is not limited to, anthryl, azulenyl, biphenyl, fluorenyl, naphthyl, phenanthryl, phenyl, pyrenyl, and terphenyl. Aryl may also mean a bicyclic or tricyclic carbon ring, where one of the rings is aromatic and the other may be saturated, partially unsaturated, or aromatic. Examples of such polycyclic aryls include but are not limited to dihydronaphthyl, indanyl, indenyl, or tetrahydronaphthyl (tetralinyl), or any of their chemically suitable substituents.

The term “bilayer” or “amphipat bilayer” or “lipid bilayer” means a molecular arrangement in which two monolayers of amphipats adhere together in a tail-to-tail fashion so that the hydrophilic “headgroups” face the polar fluid medium on either side. Any non-confined bilayer is consequently tension-free. Far from an interfering surface, a lipid bilayer typically closes into a vesicle, which is most often quasi-spherical (and typically large and thus locally quasi-planar) and only locally or exceptionally more curved, e.g. when it takes a tubular, form. A vesicle can have several bilayers.

The term “branched”, when applied to a fatty chain in the context of the invention, means a chain with at least one methyl side-group, e.g. in an iso- or anteiso-position of the fatty acid chain, but near the middle of the chain, e.g. an 10-R-methyloctadecanoic acid or tuberculostearic chain, or in several chain locations (e.g. a multi-branched meadow-foam fatty acid). The group of branched alkyls for purpose of this invention can also include isoprenoid fatty acids, such as 2,6-dimethylheptanoic to 5,9,13,17-tetramethyloctadecanoic acid and more often 3,7,11,15-tetramethyl-hexadecanoic (phytanic), 2,6,10,14-tetramethylpentadecanoic (pristanic) or 4,8,12-trimethyltridecanoic acid. Combinations of double bonds and side groups on the same hydrophobic chain can be additionally advantageous.

The terms “cation” and “cationic” means herein any positively charged atom or group of atoms, typically soluble in water and prone to migrate to a cathode in an electrolytic cell, including combinations and/or substituted forms thereof.

The term “co-solvent” herein includes but is not limited to the group of short- to medium chain alcohols, such as C1-C8 alcohols, e.g. ethanol, glycols, such as glycerol, propylene glycol, 1,3-butylene glycol, dipropylene glycol or polyethylene glycols, preferably comprising ethylene oxide (EO) units in the range from about 4 to about 16, e.g., from about 8 to about 12.

The term “fragrance” means herein any pharmaceutically acceptable compound which, if incorporated into an embodiment, assists in masking and/or improving the formulation odor. Popular examples include but are not limited to linalool, menthol, cis-3-hexene-1-ol, geraniol, neroll, citronellol, myrcene and myrcenol, nerolidol, benzaldehyde, eugenol, 1-hexanolhexyl acetate or dihydrojasmone.

The term “halo” or “halide” refers to a bromine, chlorine, fluorine, or iodine.

The term “heteroaryl” means a monocyclic aromatic group and/or multicyclic aromatic group containing at least one aromatic ring which contains at least one, but can contain several, heteroatoms selected independently from nitrogen, oxygen or sulphur. A ring of the heteroaryl group can contain 1 to 2 (1-2) oxygen atoms, 1-2 sulphur atoms, or 1-4 nitrogen atoms, or any chemically acceptable combination thereof, such that the total number of heteroatoms per ring is ≦4 with at least one C-atom per ring. The heteroaryl may be attached to the main structure at any heteroatom or C-atom providing a stable compound. Typical numbers of atoms per heteroaryl are 5-20, 5-15, or 5-10. As part of an ion of this invention, a heteroaryl typically has 5-10 atoms.

Examples of monocyclic heteroaryl groups include, but are not limited to furanyl, imidazolyl, isothiazolyl, isoxazolyl, oxazolyl, oxadiazolyl, pyrazinyl, pyrazolyl, pyrazolinyl, pyridyl, pyridazinyl, pyrimidinyl, pyrrolyl, thiazolyl, thiadiazolyl, thienyl, and triazinyl. Examples of bicyclic heteroaryl groups include but are not limited to benzimidazolyl, benzofuranyl, benzopyranyl, benzothiazolyl, benzothienyl, benzoxazolyl, chromonyl, cinnolinyl, coumarinyl, dihydroisoindolyl, furopyridinyl, indazolyl, indolyl, indolizinyl, isobenzofuranyl, isoquinolinyl, purinyl, pyrrolopyridinyl, quinolinyl, quinoxalinyl, tetrahydroisoquinolinyl, tetrahydroquinolinyl, and thienopyridinyl. Examples of tricyclic heteroaryl groups include but are not limited to acridinyl, benzindolyl, carbazolyl, phenanthridinyl, phenanthrollinyl, and xanthenyl. Chemically suitable substituents of any of the listed heteroaryls are also suitable with the disclosed formulations. Furanyl, imidazolyl, isothiazolyl, isoxazolyl, oxazolyl, pyrrolyl, thiazolyl and thienyl are especially preferred heteroaryl groups in forming an anion or a cation.

The term “heterocyclic” or “heterocyclyl” means a monocyclic non-aromatic ring system or a multicyclic ring system containing at least one non-aromatic ring in which one or more of the non-aromatic ring atoms are independently selected heteroatoms of nitrogen, oxygen or sulphur, the remaining ring atoms being C-atoms. In certain embodiments, the heterocyclyl or heterocyclic group has from 3-20, 3-15, 3-10, 3-8, 4-7, or from 5-6 ring atoms. Some rings may be partially or fully saturated, or aromatic. The heterocyclyl may be a mono-, bi-, tri-, or tetra-cyclic ring system. The heterocyclyl may include a bridged or a fused ring system, optionally containing oxidised nitrogen or sulphur atoms; the nitrogen atoms may moreover optionally be quaternised. The heterocyclyl may be attached to the main structure at any heteroatom or carbon atom providing a stable resulting compound. For purposes of ion substitution, the heterocyclyl or heterocyclic group has from 3-10, from 3-8, from 4-7, or from 5-6 ring atoms.

Examples of preferred heterocyclic radicals include, but are not limited to acridinyl, azepinyl, benzimidazolyl, benzindolyl, benzoisoxazolyl, benzisoxazinyl, benzodioxanyl, benzodioxolyl, benzofuranonyl, benzofuranyl, benzonaphthofuranyl, benzopyranonyl, benzopyranyl, benzotetrahydrofuranyl, benzotetrahydrothienyl, benzothiadiazolyl, benzothiazolyl, benzothiophenyl, benzotriazolyl, benzothiopyranyl, benzoxazinyl, benzoxazolyl, benzothiazolyl, 6-carbolinyl, carbazolyl, chromanyl, chromonyl, cinnolinyl, coumarinyl, decahydroisoquinolinyl, dibenzofuranyl, dihydrobenzisothiazinyl, dihydrobenzisoxazinyl, dihydrofuryl, dihydropyranyl, dioxolanyl, dihydropyrazinyl, dihydropyridinyl, dihydropyrazolyl, dihydropyrimidinyl, dihydropyrrolyl, dioxolanyl, 1,4-dithianyl, furanonyl, furanyl, imidazolidinyl, imidazolinyl, imidazolyl, imidazopyridinyl, imidazothiazolyl, indazolyl, indolinyl, indolizinyl, indolyl, isobenzotetrahydrofuranyl, isobenzotetrahydrothienyl, isobenzothienyl, isochromanyl, isocoumarinyl, isoindolinyl, isoindolyl, isoquinolinyl, isothiazolidinyl, isothiazolyl, isoxazolidinyl, isoxazolyl, morpholinyl, naphthayridinyl, octahydroindolyl, octahydroisoindolyl, oxadiazolyl, oxazolidinonyl, oxazolidinyl, oxazolopyridinyl, oxazolyl, oxiranyl, perimidinyl, phenanthridinyl, phenathrolinyl, phenarsazinyl, phenazinyl, phenothiazinyl, phenoxazinyl, phthalazinyl, piperazinyl, piperidinyl, 4-piperidonyl, pteridinyl, purinyl, pyrazinyl, pyrazolidinyl, pyrazolyl, pyridazinyl, pyridinyl, pyridopyridinyl, pyrimidinyl, pyrrolidinyl, pyrrolinyl, pyrrolyl, quinazolinyl, quinolinyl, quinoxalinyl, quinuclidinyl, tetrahydrofuryl, tetrahydrofuranyl, tetrahydroisoquinolinyl, tetrahydropyranyl, tetrahydrothienyl, tetrazolyl, thiadiazolopyramidinyl, thiadiazolyl, thiamorpholinyl, thiazolidinyl, thiazolyl, thienyl, triazinyl, triazolyl, and 1,3,5-trithianyl. It may be also desirable to substitute a heterocyclic radical in one or more locations in the system.*

When part of a lipid, the cyclic groups of the invention are preferably located in the middle or towards the end of the lipid chain and can include but is not be limited to cyclopropane (or cyclopropene), cyclohexyl, or cycloheptyl rings. When associated with an ion, the cyclic group of the invention is typically small (comprising preferably only one 3-7-member ring and at most two such rings) and preferably carries a large percentage of polar segments near to each other and near to the charged group(s).

The term “HLB” refers to the Hydrophilic-Lipophilic Balance number and the commonly used Griffith-nomenclature, which is used herein, and is in 0-20 range. Amphipat polarity, and thus hydrophilicity, increases with increasing HLB, and vice versa. Amphipats with a high HLB consequently disperse/form micelles in water readily; they also support oil-in water emulsion (o/w) formation. Amphipats with a low HLB, in turn, tend toward water-in-oil (w/o) emulsions or poorly hydrated inverse or lamellar phases, if they hydrate at all. (For HLB calculations see e.g., Pasquali et al., 2008, Int J Pharma 356: 44).

The HLB of many common surfactants is tabulated (see e.g. “Handbook of Pharmaceutical Excipients”; “Handbook of Detergents, Part A: Properties”; G. Broze, Ed., Marcel Dekker, New York, 1999: “Handbook of Industrial Surfactants”; M. Ash & I. Ash, Synapse Information Resources, 2008 [4th edit.]; Pasquali et al., op. cit.). Another source, is “Gardner's commercially important chemicals: synonyms, trade names, and properties”, George W. A. Milne, ed., Wiley, New York, 2005.

The term “homogeneous” means herein that a formulation or a preparation shows no visible sign of irreversible separation of the components or colloid. Unless stated otherwise, this may be confirmed visually, i.e. macroscopically. In borderline cases, where visual inspection is uncertain, more detailed investigation (e.g. using a phase-contrast or polarisation microscope) may be necessary. Non-homogeneity, if any, must be confirmed by re-inspecting the studied preparations after gentle remixing.

The term “humectant”, or moisturiser, means herein a compound that helps maintain and ideally improves hydration, e.g. of the skin. Nonlimiting examples are glycerol, propylene glycol and glycerol triacetate, butylenes glycol, other polyols (such as sorbitol, xylitol and maltitol, and polydextrose), acetamide and lactamide; natural extracts (e.g. quillaia, alpha-hydroxy acids (such as lactic acid), hyaluronic acid, pyrrolidine carboxylic acid (pyroglutamate), biphosphate, hexamethaphosphate, (tri)polyphosphates, sucrose, trehalose, and urea, or their pharmacologically acceptable salts and derivatives (such as lower-alkyl-sorbates or polyoxyethylenes, alkylated, e.g. butylated, polyoxymethylene urea, etc), and ectoin.

The term “hydroxy” in the framework of this application means a hydroxy group on a fatty acid, unless specified otherwise. Chain-lengths for the preferred hydroxy-fatty acids vary from about C10 to about C30, more preferred from about C12 to about C22, and even more preferred from about C12 to about C20. Such fatty acids are normally saturated but can also be monoenoic.

The term “inflammation” as used herein relates to any inflammatory condition, including but not limited to arthritic conditions, such as osteoarthritis and rheumatoid arthritis, the inflammatory side effects of various viral or bacterial infections, chemical, physical, or radiation-induced trauma, etc. Perhaps the most known biochemical marker of inflammation is the increased activity of cyclo-oxygenase-1, -2, or -3 and/or lipoxygenase, which can be confirmed using standard assays. On a more macroscopic level, one can alternatively measure the side effects of such an activation, including edema, erythema, hyperalgesia, algesia, and the like.

The term “ion” refers to an anion or a cation, with one, two three, four, and occasionally more, negative or positive net charges, respectively. Molecules having an unequal number of positive and negative charges may be ions for purposes of the invention as well. “Ionic”, “anionic”, “cationic”, etc. have the corresponding meaning.

The term “lipid” means herein a substance with at least one fatty segment. Each lipid of the invention thus has at least one extended lipophilic (i.e. hydrophobic and water-insoluble, apolar) group, called the “chain” or “tail” (which is often but not necessarily linear). A lipid may moreover contain at least one hydrophilic segment (i.e. lipophobic and more water-than fat-soluble, polar), termed the “headgroup”. A simple lipid can be represented with the following Formula:

Xₖ—Yₗ—Zₘ (I)

wherein at least one of the three counting-indices (k, l, m), which refers to the number of hydrophilic segments, is non-zero. The other two indices are then positive or zero. (If X and Y or Y and Z are lipophilic, lipid is of double-chain type; otherwise it is a block-copolymer.) A particularly simple lipid has one positive index (e.g. k>0 for the lipophilic tail and l=m=0 for the lacking hydrophilic headgroup) and is thus apolar. A lipid with several lipophilic chains (e.g. k>0 and l>0) is normally relatively apolar, even if it contains one small hydrophilic group (m>0). The latter in any case makes a lipid amphiphilic, namely lipo- as well as hydrophilic.

The term “lysophospholipid” (or short “lysolipid”) means a particular form of the phospholipid described by Formula (IV) below wherein a proton replaces one of the two aliphatic chains (R¹ or R²). A “lysophospholipid analogue” is a phospholipid of the Formula (IV) in which the proton is replaced by a short-chain aliphatic chain with fewer than 4 C-atoms.

The term “mammal” herein refers to any of various warm-blooded vertebrate animals of the class Mammalia, most preferably a human.

The term “membrane” is herein a synonym for the term “bilayer” or “lipid bilayer”, unless specified otherwise.

The term “molecular area” means the average area occupied by a molecule in a locally flat molecular aggregate, such as a monolayer at the air-water or air-oil interface, a large vesicle bilayer, a stack of quasi-planar bilayers, or a lamellar phase. Molecular heterogeneity (e.g. headgroups or tails distribution within the studied molecular class) can preclude a molecular area definition at a single molecule level. Moreover, even for a mono-substance the measured molecular area is nearly constant in a crystalline phase merely. Various reported or independently determined areas for the fluid-crystalline (e.g. (quasi)lamellar L-alpha phase) often differ by around 25% or more, due to changing molecular area definitions and experimental choices. However, where the Ac comparison relies on a similar definition and experimental method, the result is reasonably constant and practically useful.

A molecular area can be determined experimentally e.g. with X-ray, neutrons, or light scattering/diffraction (typically relying on the so-called Luzzati-method); using monolayer studies (e.g. in a Langmuir-Blodgett film-balance, and then typically reading-off Ac shortly before monolayer collapse, at adsorption saturation, or alternatively, and in some cases preferably, at the crystalline-liquid-condensed phase transition or even a moderately higher pressure, chosen to suit the designated final system); using an interfacial adsorption study (e.g. using the Gibb's equation to calculate the saturation area, whilst avoiding oil penetration into a monolayer at an oil-water and not at the air-water interface); using the dropping bubble or vibrating drop method (again reading-off Ac in sufficiently compact but not yet crystallised state); using NMR (if necessary using isotope-labelled chains to allow area calculation from the tail order parameter profile or value, etc. However, whichever method is chosen, one should finally compare “like with like” data, e.g. NMR data with NMR data or else correct results for experimental difference.

In addition, one may consult the published mathematical expressions relating experimental Ac values to the underlying molecular structure. Molecular area (and consequently Ac) is not a fixed number but rather a function, however, i.e. it resembles the difference between a HLB number and HLB value. It may therefore be necessary to correct a reported or calculated Ac result to account for chain-length effects, or else to determine Ac in direct comparison. The phenomenological expression Ac(nC)/A∝(n′−nC) can meet the former goal, wherein nC refers to the number of C-atoms per unit. (The proportionality factor is headgroup and potentially chains number dependent. n′ is an adjustable parameter, often between 20 and 30.) One can then start, e.g., with two well-characterised compounds (e.g. one of BL- and one of ML-type, as defined herein). One then mixes the two compounds in proportion(s) slightly above the known lamellar-to-non-lamellar (e.g. micellar) phase transition (as indicated, e.g., by the involved isotropic suspension clarity). One then diminishes relative concentration of the ML- compound in typically not more than 3-5 steps until (a turbid) lamellar/vesicular phase appears, owing to molecular area lowering (ΔAc<0), which is known for the particular compound. Subsequently, one titrates the previously uncharacterised compound to the turbid suspension in suitable aliquots to restore the original preparation transparency. From the total amphipat amount added, one calculates the solubilising molar ratio of the previously known and the newly tested, unknown, compound. Division of ΔAc with this molar ratio and addition of the known Ac of BL component leads to Ac value of the unknown compound.

The sonication method described elsewhere herein is another option for Ac assessment. In brief, one: 1 measures the time needed to make small bilayer vesicles from several different mixtures of one known and one unknown compound that together form a lamellar phase. 2. One compares the resulting experimental vesicularisation times with the vesicularisation time measured with a reference data set, e.g. in a semi-logarithmic plot (phosphatidylcholine with purity above 90% sonicated in an aqueous medium is, e.g., a useful reference). 3. One optionally checks that the tested preparation contains exclusively/predominantly, non-micellar aggregates. 4. One assigns the appropriate Ac value to the previously unknown compound via linear inter- or extrapolation. When no consensus Ac value exists, and there is no basis to select one value over the other, the smallest reliably measured Ac value is chosen.

The term “NSAID” for the purposes of this invention refers to a compound commonly recognised to be a non-steroidal anti-inflammatory drug, or class of drugs imparting an analgesic, antipyretic and/or anti-inflammatory effects. Such compounds typically act as non-selective inhibitors of the enzymecyclooxygenase, e.g. the cyclooxygenase-1 (COX-1) and cyclooxygenase-2 (COX-2) isoenzymes and include, but are not limited to salicylates, arylalkanoic acids, 2-arylpropionic acids (profens), N-arylanthranilic acids (fenamic acids), oxicams, coxibs, and sulphonanilides.

The term “oil” means herein, first, the group of fatty acid esters of polyols, such as liquid triglycerides from natural sources, including but not limited to avocado oil, bergamot oil, borage oil, cade oil, Camelina sativa oil, caraway oil, castor beans oil, cinnamon, coconut, corn, cotton and grape seeds oil; evening primrose, hazelnut, hyssop, jojoba, linseed and marrow oil; Moring a concanensis and meadowfoam oil; olive, palm kernel, peanut, primula and pumpkin oil; rapeseed or canola, saffron (safflower), sesame, soybean and sunflower oil; sea buckthorn and various fish oils, chicken fat, purcellin oil and tallow; plant and animal oils of formula R₉—COOR₁₀, in which R₉ is chosen from fatty acid residues comprising from 7 to 29 C-atoms and R₁₀ is an aliphatic chain comprising from 3 to 30 C-atoms, such as alkyl and alkenyl, e.g.; glyceryl tricaprocaprylate; a natural and synthetic essential oil, such as, e.g., eucalyptus, lavandin, lavender, vetiver, Litsea cubeba, lemon, sandalwood, rosemary, camomile, savory, nutmeg, orange and geraniol oil, or a synthetic oil defined further in the text.

Second, the term oil can refer to a mineral or synthetic oil. The former group includes alkanes ranging from octane to hexadecane, and liquid paraffin. Synthetic oils include fluorinated oils (e.g. fluoroamines, such as perfluorotributylamine), fluorohydrocarbons (e.g. perfluorodecahydronaphthalene), fluoroesters and fluoroethers, as well as lipophilic esters of at least one mineral acid and of at least one alcohol or else liquid carboxylic acid esters or volatile and non-volatile silicone oils. The synthetic oils suitable for the invention may also be chosen, e.g., from polyolefins, such as poly-a-olefins, e.g. poly-a-olefins from the classes of hydrogenated and nonhydrogenated polybutene poly-a-olefins, such as hydrogenated and nonhydrogenated polyisobutene poly-a-olefins.

A third group of oils suitable for purposes of the invention are volatile and non-volatile silicone oils, which can be combined with oil(s) lacking Si-atoms. When used, the total amount of silicone oils ranges from 5-50 wt.-% relative to total oil weight.

The term “pharmacologically acceptable” herein means that a compound, a preparation, an analytical or manufacturing method has already received or else is eligible for receiving marketing authorisation approval by a competent regulatory authority, such as the US Food and Drugs Administration (FDA), the European Medicines Agency (EMEA), the corresponding Swiss authority (Swissmedic) or the like. The preparation or component should ideally be free from unacceptable biological effects, such as irritation at the site of application or elsewhere in body, which can be confirmed using conventional methods known to the skilled person.

The term “pharmacological agent” means herein a substance or a combination of substances that is/are registered as pharmaceutically active agent(s) by a competent regulatory authority for use in or on mammals for any or for the specified indication(s), as the case may be.

The term “phase diagram” in the context of this application means a ternary, or pseudo-ternary, quaternary or pseudo-quaternary, and rarely quinternary phase diagram. Typically, such a phase diagram pertains to only one or a few temperatures, but can encompass a broader range of temperatures. If no suitable phase diagram is available, a person skilled in the art will know how to construct one using standard laboratory procedures including but not limited to polarizing microscopy, spectroscopic, and in rare cases, scattering methods. To generate an acceptable phase diagram, it may suffice to inspect preparations optically (e.g., under a microscope) after a proper equilibration, which can be accelerated by transient heating, stirring, or centrifugation.

The term “polar fluid” refers to a substance that flows under a directed stress, such as a protic fluid, e.g. water, ethyleneglycol, glycerol, or at least a medium that may homogeneously mix with water, which adequately supports the amphipat(s) suspensions and adaptable vesicles formulations of the invention.

The term “polarity units number”, or nP, defines herein the number of at least partially hydrophilic repetitive units, typically within the polymeric polar headgroup of an amphipat, which corresponds to one oxyethylene (EO) unit in the polar headgroup attached to a linear-chain polyoxyethylene (PEG)-fatty-ether. Amphipats of the Formula (Ila) have thus, by definition, n polarity units per head when R″ is a hydrogen atom; a fatty alcohol consequently carries no polarity unit. Each carbonyl group or nitrogen atom at the headgroup attachment site(s) reduces the nominal polarity units count by around −0.5. Each oxypropylene segment corresponds to around ⅓ polarity units. Each oxyethylene or oxypropylene segment attached stochastically to a sorbitan-ring that is also coupled to at least one fatty residue (as in the amphipats of the Formula (IIb)) contributes effectively 0.59/n polarity units to the headgroup attached to n hydrophobic chains. E.g., polysorbate 80 (with nEO=20 and R=C18:1, R′=R″=protons, i.e. n=1) has a similarly polar headgroup as a linear PEG-ether with the same hydrophobic chain length and nEO 11.8; polysorbate 85 (with nEO=20 and R=R′=R′=C18:1, i.e. n=3) roughly corresponds to a linear PEG-ether with nEO˜3.9 and C18:1. Neglecting possible sugar stereochemistry effects, a mono-aliphatic hexose-ester or -amide carries around 3.8 polarity units. Most commercial sugar-derivatives have n>1 hydrophobic chains attached to each sugar residue, however. This affects the resulting amphipat polarity, which is then “distributed over” n chains, giving nP˜3.8/n as the effective polarity units count. The second sugar segment in a headgroup (as in maltose vs. glucose) typically increases the effective polarity units number with lesser effect, normally by no more than around 10-20%.

A polyglyceride polarity units number is also sensitive to distribution and total number of hydrophobic chains on the headgroup and ranges from around 1.65 for an essentially linear mono-aliphatic-oligo- or -polyglyceride through 0.8 down to around 0.2 polarity units per C18:1 hydrocarbon chain in a stochastic oligo-fatty-ester-oligo- or -polyglyceride. (A commercial fatty-pentaglyceride thus can correspond to a PEG-fatty-ether with nEO˜3 and its nominally similar kin from a different manufacturer to a PEG-fatty-ether with nEO˜0.3). N,N-dimethylamine-N-oxide corresponds to around 5 polarity units. A glycerophosphocholine or a charged, but electrostatically screened, glycerophosphoglycerol on a double-chain lipid correspond to around 2 polarity units per fatty chain and to around 4.5 units per hydrocarbon chain of the corresponding lysophospholipid. A double-chain glycero-phosphate-monomethyl-ester or glycerol-phosphoethanolamine-(N,N)-dimethyl carry around 1.4 polarity units per fluid fatty chain each. The corresponding mono-charged, but screened, phosphatidic acid contributes zero polarity units to a bilayer, which is thus controlled only via chains. Exchanging a phosphate headgroup on an amphipat with a sulphate group does not affect the resulting molecular polarity. Based on these values, one can assign polarity unit equivalents to other relevant headgroups based on published, or otherwise readily obtainable, information.

The term “polysiloxane” is herein synonymous with “silicone”.

The term “preferred chain(s)” means herein one or more acyl, alkyl, alkenyl, alkynyl or alkenoyl hydrocarbon radical(s) with C8 to C24, more likely with C12 to C22, preferably with around C14 to around C20. Any preferred chain should be disordered (i.e. a fluid) at least at body surface temperature (i.e. typically around 30-32° C. and more broadly between 25° C. and 37° C.). However, chain fluidity above 0° C. is desirable. As the preferred chains should resemble chains, or at least chain lengths, that are prevalent in skin tissue, fluid C18 or C20 chains at the specified temperature range are the most preferred for the purposes of the invention. Side-chains, such as branches, or side-groups, including oxo-residues, or double bonds, especially in cis-configuration, promote hydrocarbon chains fluidity. The number of side-chains, side-groups, or double bonds per chain is ideally 1-3, lower number of double bonds being preferable. This includes, but is not limited to, the particularly useful mono-unsaturated oligo-alkenoyls with 18 C-atoms per radical cis-6-octadecenoic (=petroselinic=6c-18:1=C18:1(n-12)), cis-9-octadecenoic (=oleic=9c-18:1=C18:1(n-9)) and cis-11-octadecenoic (=cis-vaccenic=11c-18:1=C18:1(n-7)), in addition to desirable di-unsaturated 9-cis,12-cis-octadecadienoic (=linoleic or gamma-linoleic=9c,12c-18:2=C18:3(n-6)), 15-cis-octadecadienoic (=alpha-linoleic=12c,15c-18:2=C18:2(n-3) 12-cis). The preferable shorter mono-alkenyl is C16:1(n-9) or a palmitoleic chain. The preferred longer mono-alkenoyls are of the cis-11-eicosenoic or gondoic and 11-cis,14-cis-eicosadienoic (=11c,14c-20:2=C20:2(n-6)) type.

Potentially useful but less attractive for the invention are the tri-unsaturated linolenic 6-cis,9-cis,12-cis-octadecatrienoic (=gamma-linolenic=6c,9c,12c-18:3=C18:3(n-6)), 9-cis,12-cis,15-cis-octadecatrienoic (=alpha-linolenic=9c,12c,15c-18:3=C18:3(n-3)) or else 8-cis,11-cis,14-cis-eicosatrienoic (or dihomo-gamma-linolenic=8-c,11-c,14-c-20:3=20:3(n-6)) chains, due to their oxidation sensitivity, or else chains with the double bonds in trans-configuration, such as 9t-18:1=trans-9-octadecenoic (=elaidic) and 11t-18:1=trans-11-octadecenoic (=vaccenic)), due to relatively poor biotolerability. For similar reasons, the alpha-variants are more preferred than the gamma-variants, and the 5c,8c,11c,14c-20:4=C20:4(n-6)=5-cis,8-cis,11-cis,14-cis-eicosatetraenoic (or arachidonic) chain type should be avoided owing to its pro-inflammatory activity. The 15-hydroxy-hexadecanoic and 17-hydroxy-octadecanoic ricinoleic, i.e. D-(−)12-hydroxy-octadec-cis-9-enoic chains also deserve special consideration for purposes of the invention. The latter type of chains typically comes from castor oil and may be used in hydrogenated form.

The term “range” used in connection with numerical values means that the numerical value can be any value in said range. For the purposes of this invention, it also means that within the broadest range specified, any narrower range can be chosen using 50%, 33%, 25%, 22.5%, 20%, 17.5%, 15%, 12.5%, 10%, 9%, 8%, 7%, 6%, 5%, 4%, 3%, 2%, or 1% of the entire range. For example, a range of 1 to 10 is thus divisible and/or limited to 1 to 2, 2 to 3, 3 to 4, 4 to 5, 5 to 6, 6 to 7, 7 to 8, 8 to 9 and 9 to 10 or else to 1 to 3.33, 3.33 to 6.66 and 6.66 to 9.99 or 3.33 to 9.99, or from 1 to 4, 4 to 7, 7 to 10, 1 to 7 or 4 to 10; or else from 1 to 3.25, from 3.25 to 5.5, from 5.5 to 7.75, from 7.75 to 10, from 1 to 5.5, from 1 to 7.5, from 3.25 to 7.5 from 3.75 to 10, or from 5.5 to 10.

The term “simple or complex, organic or inorganic salt” means herein an anion or a cation. Common exemplary anions include dissociated acids, hydroxy-acids, halides (such as chloride, bromide, and iodide), nitrates, phosphates, or alkyl phosphates or alkyl aryl phosphonates, alkyl sulphates (such as methyl sulphate), alkyl sulphonates (such as methanesulphonate) and alkyl aryl sulphonates. Cations include but are not limited to alkali or alkali earth ions, various amines, etc. Combinations of a plurality of simple or complex, organic or inorganic salts are also contemplated.

The term “substituent” or “substitute” indicates that a group, including alkenyl, alkyl, alkynyl, aralkyl, aryl, cycloalkyl, heterocyclyl, and heteroaryl, may be optionally substituted with typically 1 to 4 substituents. For any heteroaryl, one, two, three or four substituents are independently selected from the group consisting of cyano, halo, oxo, nitro, C₁₋₆ alkyl, halo-C₁₋₆ alkyl, C₂₋₆ alkenyl, C₂₋₆ alkynyl, C₃₋₇ cycloalkyl, C₆₋₁₄ aryl, C₇₋₁₄ aralkyl, heteroaryl, heterocyclyl, —C(O)R′, —C(O)OR′, —C(O)NR″R′″, —C(NR′)NR″R′″, —OR′, —OC(O)R′, —OC(O)OR′, —OC(O)NR″R″, —OC(═NR′)NR″R′″, —OS(O)R′, —OS(O)₂R′, —OS(O)NR″R′″, —OS(O)₂NR″R′″, —NR″R′″, —NR′C(O)R″, —NR′C(O)OR″, —NR′C(O)NR″R′″, —NR′C(═NR″″)NR″R′″, —NaS(O)R″, —NR′S(O)₂R″, —NR′S(O)NR″R′″—NR′S(O)₂NR″R′″, —SR′, —S(O)R′, —S(O)₂R′, and —S(O)₂NR″R′″, wherein each a, R″, R′″, and R″″ independently hydrogen, C₁₋₆ alkyl, C₂₋₆ alkenyl, C₂₋₆ alkynyl, C₃₋₇ cycloalkyl, C₆₋₁₄ aryl, C₇₋₁₄ aralkyl, heteroaryl, or heterocyclyl; or R″ and R″ together with a nitrogen atom, to which they are attached, form a heterocyclyl.

The term “sufficient”, when used in the context of adaptability or stability tests, means that the experimental test result falls within ±50%, preferably within ±33%, more preferably within ±25%, most preferably within ±20% of acceptable error limits.

The terms “therapeutically effective” or “therapeutic effect” mean that the effect of an application of any of the claimed formulations on a mammalian, human or animal, body, is deemed to be beneficial enough to the treated subject to warrant additional applications of the formulation on the same or different subject. The conclusion is typically based on observation of an appreciable alleviation, decrease and/or mitigation of at least one clinical symptom by the treatment. Clinical symptoms associated with the conditions claimed to be treatable by the methods of this invention are well-known. Further, those skilled in the art will appreciate that the therapeutic effect(s) need not be complete or curative, as long as the benefit provided to the treated subject is meaningful from the standpoint of supervising individual or a person applying said treatment.

The term “thickener” means any pharmaceutically acceptable substance, or a mixture thereof, that increases the viscosity of a given formulation to a desired level. Examples include, but are not limited to, pharmaceutically acceptable hydrophilic polymers, such as partially etherified, semi-synthetic cellulose derivatives (e.g. carboxymethyl-, hydroxyethyl-, hydroxypropyl-, hydroxypropylmethyl- or methylcellulose, of which hypromellose (INN), short for hydroxypropyl methylcellulose (HPMC) and methyl cellulose find broadest usage); fully synthetic hydrophilic polymers (such as polyacrylates (a leading trade mark: Carbopol® (Gattefosse), polymethacrylates, poly(hydroxyethyl)-, poly(hydroxypropyl)-, poly(hydroxypropylmethyl)methacrylate; polyacrylonitrile/methallyl-sulphonate; polyethylenes; polyoxyethylenes; polyethylene glycols; polyethylene glycol-lactides; polyethylene glycol-diacrylate; polyvinylpyrrolidone; various polyvinyl alcohols; poly-(propylmethacrylamide), poly(propylene fumarate-co-ethylene glycol; polyaspartamide; (hydrazine cross-linked) hyaluronic acid; natural gums (including agarose, alginates, carrageenan, chitosan, collagen, gelatin, guar-gum, (amidated) pectin, tragacanth, and xanthan); silicone; as well as pharmaceutically acceptable and practically useful mixtures and further derivatives or co-polymers of such compounds.

The term “vesicularisation time” is herein defined as the time required to transform an originally opaque suspension (i.e. optical density>>3) to an opalescent/transparent suspension with a much lower optical density via external stress, e.g. generated with an ultrasound transducer, high-shear homogeniser (Ultra-Turrax®, IKA) or rotor-stator homogeniser. For comparative purposes, the final optical density can be chosen arbitrarily, so long as it is at least 3-4-times lower than the starting optical density, and the compared suspensions are tested under similar conditions in terms of total amphipat concentration, temperature, total volume, etc. Transformation into small vesicles can be identified, roughly, with the final optical density of a non-absorbing sample around 0.8±0.4 (1 cm light-path; 800 nm incident light wavelength).

A person skilled in the art can readily prepare equivalents to the specific formulations and procedures used in the present invention. Such equivalents therefore fall within the contemplated scope of the invention and the claims. The contents of all cited references, patents, and patent applications are hereby incorporated by reference. The appropriate components, processes, and methods of these cited disclosures may be suitably selected for use in the embodiments of the present invention.

### 5.1. Amphipats, Amphiphiles, Lipids

Any amphipat with sufficiently prominent hydrophobic segment capable of aggregation into an entity that is not merely a small oligomer can be a lipid according to the invention. To characterise and well differentiate between the potentially useful lipids, it is advantageous to distribute them in 3 classes: i) monolayer and micellar phase or (quasi)isotropic aqueous “bulk phase” formers (“ML”); ii) bilayer and lamellar phase formers (or “BL”); and iii) inverse-micellar and (quasi)isotropic (oily) bulk phase formers (or “IM”). These three classes can be related to the average area per chain (“Ac”):

ML:Ac/nm²>0.35-0.50 (for large molecules up to 0.55);

BL:0.18-0.28≦Ac/nm²≦0.35-0.50; and

IM:Ac/nm²≦0.18-0.22 (in a gel phase), 0.26-0.30 nm² (in a fluid phase).

The need to specify the lower and upper bounds instead of a single value, which would ideally be in the range around 0.45 nm² for the fluid phase BL-amphipats and around 0.195 nm² (ordered chains) or around 0.28 nm² (fluid chains) for IM amphipats, is partly due to the described definitions and experimental variability. Another reason relates to the diversity of molecular packing, especially in amphipat mixtures, which typically causes single-chain amphipats to have Ac values in the upper part of the described ranges, and double-chain amphipats to tolerate a broader range of Ac values, including such in the lower part of the specified ranges. Further reasons are the intrinsic polydispersity of molecules with multiple hydrophobic chain and/or polar-groups, as well as the fact that commercial products may contain undesirable traces of unreacted or improperly reacted molecular components.

The molecular area requirement of ML- and BL-class amphipats is mostly, but not entirely, governed by the hydrated headgroup effective size. One can calculate the Ac from the molecular structure (duly allowing for the system's main sensitivities to molecular heterogeneity and the boundary conditions).On the calculated Ac basis one can then select the amphipats meeting the bilayer formation criteria defined in the previous text. Similar rules and limiting Ac estimates apply to molecular mixtures, individual (average) Ac values in the first approximation then adding-up in relative molar proportions. For a mixture of two components with areas Ac1 and Ac2, blended in molar fractions x1 and x2, for example, the result is approximately Ac,mix˜Ac1*x1+Ac2*x2, assuming a uniform mixing of both components and headgroups. Increasing the molecular mismatch between different components or their segments within a bilayer creates an unstable bilayer conformation, by widening the interface, which causes a negative deviation from the calculated average molecular, if not phase separation.

It is moreover permissible, in the first approximation, to identify the effective polarity units number and the effective HLB number of a mixture with the appropriate weighted averages. For two types of amphiphiles with HLB1 and HLB2 combined in molar fractions x1 and x2, e.g., this means that HLBmix˜HLB1*x1+HLB2*x2. This formula, however, only suitably applies to the mixed amphipats with sufficiently similar structure, chain length, and/or degree of unsaturation and/or branching, to ensure at least the desired quasi-uniform molecular mixing.

Addition of ML-class lipids to BL-class lipids pushes the mixed aggregates toward a micellar configuration, i.e. out of the bilayer region, and vice versa. In turn, the addition of IM-class to BL-class lipids increases the propensity for bilayer diversion into an inverse non-lamellar form (which can still contain bilayer-like segments or else be bicontinuous, e.g.). Balanced addition of ML- and IM-lipids to BL-class lipids is normally effect-neutral, assuming a uniform distribution of additives.

### 5.1.1 Amphipat Aggregation

One or more embodiments of the present invention involve fatty esters or ethers of non-ionic polyethylene glycol (i.e. PEG)=polyoxyethylene=polyethylene-oxide (i.e. poly-EO) with a strong aggregation tendency. The most common ether-type amphipats have the general Formula:

R′—(O—CH₂—CH₂)ₙ—OR″ (IIa)

in which R′ is an aliphatic tail comprising from about 8 to about 30 C-atoms; R″ is a hydrogen atom, a linear or branched, saturated or unsaturated alkyl group with from about 1 to about 30 C-atoms, or a linear or branched, saturated or unsaturated alkyl or alkenyl group with from about 1 to about 30 C-atoms. n is a number in the range from 1 to about 150.

In one embodiment, the compound of Formula (IIa) is a BL-class lipid with R′ being an alkyl or alkenyl group with from about 8 to about 24 C-atoms, preferably from about 12 to about 22 C-atoms, and most preferably about 18 C-atoms. R″ is then typically hydrogen or a lower chain alkyl, in particular a methyl. n≡nEO ranges from 1 to about 150, preferably from about 2 to about 20, and even more preferred from about 3 to about 8, depending on aliphatic chain length as explained below. In another exemplary embodiment R″ is an alkyl or alkenyl group with from about 8 to about 24 C-atoms, preferably from about 12 to about 22 C-atoms, and more preferably about 18 C-atoms. nEO then ranges from about 4 to about 150, preferably from about 6 to about 40, and even more preferred from about 7 to about 20, again depending on the chosen aliphatic chain-length. The preferred double-chain BL-class lipids have about 8 to around 12-14 PEG units per headgroup, often with a broad distribution, smaller values typically pertaining to shorter chains. Higher nEO-values correspond to ML-class amphipats:two C18 chains should be coupled to nEO>12-14 and typically nEO 16 to yield a ML-class lipid.

More specifically, when R′ is a linear, fully saturated chain with 12 C-atoms a surfactant of the Formula (IIa) is a BL-class lipid if 2.5≦nEO≦3 (or at most 4.25). The limiting nEO value increases moderately with increasing C-atoms number in R′ and decreases, but less strongly, with aliphatic chain unsaturation, branching, or derivatisation. The acceptable range of nEO values for a BL-class oleyl-EOₙ ether is e.g. 3.5-7.5. At higher temperatures, which lower the average area per chain and HLB value, moderately higher relative nEO values may be acceptable. Higher than the specified, BL-ensuring, nEO values afford ML-like lipids (which destabilise bilayers but can stabilise aggregates in a suspension). Such destabilising effect can be compensated, where necessary, by combining a high nEO value amphipat with an amphipat having a longer and potentially less unsaturated chain and a similar headgroup, or with an amphipat having a similar aliphatic chain and a shorter headgroup, to yield a tolerable overall average nEO value. Moreover, the relative hydrophobicity can be boosted without headgroup shortening or aliphatic chain prolongation by e.g. halidation or silanisation. Trisiloxane surfactants, often denoted as M(D′EOₙ)M (where M means the trimethylsiloxy group, (CH₃)₃—SiO_{1/2}— and D′ means —O_{1/2}Si(CH₃)(R)O_{1/2}—, where R is e.g. a polyoxyethylene group attached to the silicon through a propyl spacer), tend to form spontaneously a lamellar phase from which bilayer vesicles can be made in presence or absence of an oil. M(D′EO₆)M is an example.

Linear fatty PEG esters resemble molecules of the Formula (IIa) and obey qualitatively similar rules of selection as are specified in the previous paragraph. Quantitatively, however, they require around 10-20% longer headgroups to match the packing properties of their ether-bonded relatives.

The indirect PEG-esters (i.e. polysorbates) can have the general Formula:

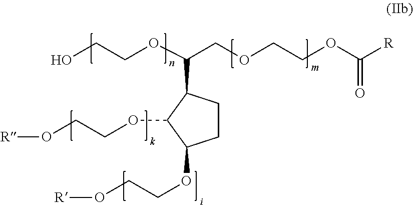

wherein R is a linear or branched, saturated or unsaturated fatty residue, which is ideally a preferred chain as defined herein. R′ and R″ are each either a proton or a fatty residue, in the latter case ideally a preferred chain. i, k, n, m are integer numbers, wherein l, k, n, can be zero as well (as in Span series). For this class of molecules, nEO is the sum l+k+n+m, and should consequently be higher than previously specified to match properties of the molecules of the Formula IIa. The needed “excess” is often around 60-90% (dependent on molecular purity), and tends to increase with nEO value.

Instead of polyethyleneglycol, polypropyleneglycol (PO or PPG) groups can be used that are coupled directly or indirectly to at least one fatty chain. PPG is less polar than PEG, causing the optimum number of PPG units per headgroup to exceed the optimum number of PEG units specified previously, if otherwise similar molecules are used. Also suitable are the relatively simple block-co-polymers with one or more polyoxypropylene (PPG) chains attached anywhere in or between the oxyethylene chain and the hydrophobic anchor. One preferred option is to insert a PPG segment between the hydrophobic R′ and polyoxyethylene chain in compounds derived from the original Formula (I). This brings the technical advantage of enlarging the amphipat area per chain whilst decreasing rather than increasing molecular hydrophilicity.

Instead of aliphatic chains, (poly)cyclic, such as aryl and heteroaryl, segments or a mixture of aliphatic and cyclic or aromatic groups can be used to anchor polar, e.g., PEG or PPG, oligomers or polymers into aggregates. Non-limiting examples include the water soluble tocopheryl PEG glycol esters or tocopheryl PEG glycol succinic acid esters.

PEG-aryl-ethers are typically employed mainly in industry and in biochemistry applications. However, PEG-aryl ethers are in principle useful for the invention as well. The preferred aryl groups in surfactants of the class are octylphenol, nonylphenol, decylphenol, dodecylphenol, or dinonyl.

PEG-glycerol-esters or PPG-glycerol-esters are another class of amphipats suitable for the invention. PEG/PPG-glycerol-monoesters are relatively more water soluble than the direct fatty PEG/PPG esters, on equal PEG/PPG-number and chain-length/type basis. The commercial PEG- or PPG-glycerol-esters are typically mixtures of mono-, di-, and triacyl derivatives, however, which makes them relatively less polar. Manufacturer specifications may be considered in selecting suitable PEG-glycerol-ester or PPG-glycerol-ester for purposes of the invention.

Lipophilic polyglyceryls, such as the intermediate to long chain poly-glyceryl-fatty esters, ethers, amines, or polyglyceryl N-fatty acyl aminoacid esters are particularly useful for purposes of the invention, owing to their biological origin and small temperature sensitivity. The BL-class molecules of this kind have typically 2 to 3 repetitive units per chain, but can carry many more in the multi-chain compounds. Suitable members of the group thus include but are not limited to 2-, 3-, 4-, 5-,6-, 7-, 8-, 9-, or even 10-glyceryl-derivatives with at least one acyl, alkenyl, alkyl, alkynyl, aralkyl, aryl, cycloalkyl, heterocyclyl, heteroaryl, or any other biologically acceptable chain, whether the latter is straight or branched, saturated or unsaturated. The alternative N-fatty acyl-neutral amino acids have mostly 12-C22 C-atoms per hydrophobic chain. The neutral amino acid may be any short chain (i.e., C2 to C4) amino acid such as alanine, beta-alanine, aminobutyric acid, alpha-aminobutyric acid, glycine, glutamic acid, N-methyl-beta-alanine, and, preferably, N-methyl-glycine. When using the latter, the long chain acyl group is N-fatty acyl-sarcosyl, and the polyglyceryl ester is a polyglyceryl N-fatty acyl-sarcosinate. Accordingly, examples of suitable polyglyceryl N-fatty acyl amino acid esters include, but are not limited to, polyglyceryl-acyl-sarcosinates.

Also suitable for the invention are the mixed esters derived from (i) at least one fatty acid, at least one carboxylic acid, and glycerol, and the mixed esters derived from (ii) at least one fatty alcohol, at least one carboxylic acid, and glycerol, wherein said at least one carboxylic acid is chosen from the class of hydroxy acids and succinic acid, including, e.g., (i) mixed esters derived from at least one fatty acid comprising at least one alkyl chain ranging from about C8 to about C30, at least one a-hydroxy acid, and glycerol; (ii) mixed esters derived from at least one fatty acid comprising at least one alkyl chain with about 8 to about 30 C-atoms, succinic acid, and glycerol; (iii) mixed esters derived from at least one fatty alcohol comprising at least one about C8 to about C30 alkyl chain, at least one a-hydroxy acid, and glycerol; and (iv) mixed esters derived from at least one fatty alcohol comprising at least one alkyl chain comprising about C8 to about C30 chains, succinic acid, and glycerol. The alpha-hydroxy acid can be, e.g., citric acid, lactic acid, glycolic acid, malic acid, etc.

Additional C3-C8-alkylene triol-ethers or -esters include mixed ethers or esters, i.e. components including other ether or ester ingredients, e.g. transesterification products of C3-C8-alkylene triol esters with other mono-, di- or polyols. Particularly suitable alkylene polyol ethers or esters include the mixed C3-8-alkylene triol/poly-(C2-4-alkylene) glycol fatty acid esters, especially the mixed glycerol/poly-ethylene- or polypropylene-glycol fatty acid esters. Suitable alkylene polyol ethers or esters include products obtainable by transesterification of glycerides, e.g. triglycerides, with poly-(C24-alkylene) glycols, e.g. poly-ethylene glycols and, optionally, glycerol. Suitable polyglycerol ethers are preferably aliphatic ethers, characterized by a high proportion of linear (i.e., acyclic) monoaliphatic compounds (i.e. oligoglycerol-mono-aliphatic ethers, such as diglycerol-, triglycerol-, tetraglycerol-, and potentially pentaglycerol-fatty ether, often of a preferred chain);
tetra- to deca-glycerol dialiphatic ethers are interesting for the invention too, as are decaglycerol trialiphatic ethers and higher polyglycerol polyaliphatic ethers.

Esters of propylene glycol and fatty acids may be suitable surfactants for the invention if their area per chain or nP or HLB value is properly chosen. However, most commercial surfactants of this class have insufficient Ac, and thus nP or HLB values.

A further suitable amphipats class are sugars, including pentoses, hexoses, homo- or hetero-di-, -tri-, or -tetra-hexoses, and of the corresponding heptoses, or their lactones. Any such polar headgroup can be substituted with alkyl, alkenyl, alkynyl, aralkyl, aryl, cycloalkyl, heterocyclyl, heteroaryl-chains, or some other pharmaceutically acceptable hydrophobic anchor, via an ester, ether, thioester, or amide bond, e.g. The attachment may be direct (as, e.g., in alkyl-alpha- or -beta-D- or -L-glucoside; in alkyl-lactoside, -maltoside, -saccharosid or -sophoroside (in lactone or acid form); in alkyl-lactobionamide or -maltobionamide, etc.) or else indirect (especially when several hydrophobic chains are attached, e.g., through a shared glycerol backbone, as in 1,2-0-diacyl-3-0-β-D-glucosy/-sn-glycerol). The sugar may also be substituted, and then contain, e.g., an amino or sialic group. Possible groups thus include glucosides, -galactosides, -maltosides, -fucosides, -fructosides, -sucrosides (i.e. -saccharosides), such as beta-D-glucopyranoside or D-maltopyranoside, but the L-forms of said carbohydrates are acceptable as well. A general Formula for an alkyl saccharide is:

(R′—Z)ₘ—R″ₙ (III)

R′ is a hydrophobic group, such as a linear or branched aliphatic chain with 8-30 C-atoms and 0-5 double bonds, optionally substituted by one or more aromatic, cyclo-aliphatic or hydrophilic groups. R″ is a group derived from any saccharide containing 4-7 C-atoms. Z is either —O—, a carboxyl-, amide-, phosphate-, or sulphide-group to which R″ is covalently bound; n is an integer from 1-10 and m is an integer smaller than the number of —OH groups on R″. Controlling the number of hydrophobic or partially hydrophobic chains attached to each sugar residue is important, as can be observed from specific examples disclosed in U.S. Pat. No. 7,008,930. Such control allows beneficially keeping the relative polarity and area per chain of the amphiphile in the desired range, greater m/n ratio normally producing a lower Ac value.

Typical sugar-based surfactants are sucrose-based. This includes but is not limited to palmitate, which is slightly above the ML/BL borderline, and sucrose-dipalmitate, which is normally a BL-type amphipat. Sugar lipids with relatively short hydrophobic chains like octyl- to dodecyl-alpha- or -beta-glucoside chains, e.g., are ML-lipids and thus membrane destabilisers and/or solubilisers.

Also useful in the invention are thioglucosides, such as alkylthioglucosides, including but not limited to those with about C10 to around C24 aliphatic-chains. The corresponding long, straight or branched, saturated or (poly)unsaturated fatty chain derivatives of 2,3,4,6-tetra-O-acetyl-b-D-glucopyranosyl ethylxanthate, 1-thio-b-D-glucose, 2,3,4,6-tetra-O-acetyl-1-thio-b-D-glucopyranoside and 2,3,4,6-tetra-O-acetyl-1-thio-b-D-galactopyranoside are also useful in the invention.

The fatty alcohol ethers of sugars useful as surfactants of the invention may be chosen, e.g., from ethers of at least one C8-C30 fatty alcohol and of glucose, of at least one C8-C30 fatty alcohol and of maltose, of at least one C8-C30 fatty alcohol and of sucrose, and of at least one C8-C30 fatty alcohol and of fructose, and ethers of at least one C14-C30 fatty alcohol and of methylglucose. A non-limiting example of such ether is alkylpolyglucosides. Further non-limiting examples include alkylglucosides, such as decylglucoside and laurylglucoside.

Well known and often preferred vesicle forming substances are the double-chain amphipats. The group includes but is not limited to double chain polyglycerides, double chain polyethyleneglycols, double chain sugar lipids (such as digalactosyldiacylglycerols) and the commonly double-chain phospholipids. The related sulpho- or arseno-lipids may be suitable for use with the invention as well.

Glycerophospholipids are generally describable with the Formula:

R¹—CH₂—CHR²—CR³H—O—XR⁵O—O—R⁴ (IV)

wherein R¹ and R² are typically, and independently, aliphatic chains (most often derived from a fatty acid or a fatty alcohol) but cannot both be hydrogen, OH or a C₁-C₃ alkyl groups; acyl or alkyl, n-hydroxyacyl or n-hydroxyalkyl, or branched chains are most preferred. R³ is generally hydrogen. R⁵ is either an —OH or an ═O group. X is typically phosphorus or sulphur, but could also be an arsenic atom. The OH-group of the phosphate/sulphate/arsenate is a hydroxyl radical or hydroxyl anion (i.e., hydroxide) form, depending on the group ionisation degree. Furthermore, R⁴ may be a proton or a short-chain alkyl group, substituted by a tri-short-chain alkylammonium group, such as a trimethylammonium group, or an amino-substituted short-chain alkyl group, such as 2-trimethylammonium ethyl group (cholinyl) or 2-dimethylammonium short alkyl group.

The related sphingophospholipids, in which sphingosine replaces glycerol as the bridging segment, have the general Formula:

R¹-sphingosine-O—XHO₂—O—R⁴ (V)

wherein R¹ is a fatty-acid attached via an amide bond to the nitrogen of the sphingosine and R⁴ as well as X have the same meaning as in Formula (IV). R¹ and R² of the Formula (IV) can be similar or different and R¹ and R² of the Formula (IV) and R¹ of the Formula (V) can be of the acyl, alkenyl, alkyl, alkynyl, aralkyl, aryl, cycloalkyl, heterocyclyl, heteroaryl, or any other biologically acceptable type. The chains for the radicals R¹ and R² of the Formulae (IV) or (V) may be selected from the class of preferred chains as defined herein. In short, R¹ and/or R² in Formulae (IV) or (V) are acyl or alkyl, n-hydroxyacyl or n-hydroxyalkyl, or branched chains with one or more methyl groups attached at almost any point of the chain (usually and preferably, the attachment point is near the end of the chain, however, in the iso- or anteiso-configuration). The radicals R¹ and R² may either be saturated or unsaturated (mono-, di- or poly-unsaturated, or branched). R³ is hydrogen and R⁴ is 2-trimethylammonium ethyl (the latter corresponding to phosphatidylcholine head group) or 2-dimethylammonium ethyl (less preferably 2-methylammonium ethyl or 2-aminoethyl, in the latter case giving phosphatidylethanolamine head group). R⁴ may also be a proton, a short chain alkyl, such as methyl or ethyl, a serine, a glycerol, inositol, or an alkylamine group. Phosphatidylethanolamine analogues can carry one or two methyl groups on terminal amine. Additional polar phosphate or sulphate esters (i.e. other radicals, R⁴) having a preference for bilayer formation and alternative chain types attached to such headgroups are described herein.

A preferred uncharged (zwitterionic) phospholipid of the Formula (IV) is phosphatidylcholine. R⁴ in the Formula (IV) is then 2-trimethylammonium ethyl and R¹ and R² are two similar or dissimilar aliphatic or cyclic (and even aromatic) chains. Natural phosphatidylcholine is preferably used in purity above 50%, more often above 70% and preferably above 80%. It may be advantageous to use phosphatidylcholine with purity above 90% or even above 95%. Another zwitterionic phospholipid suited particularly for epicutaneous applications is sphingomyelin (cf. Formula V), which can, e.g., be extracted from eggs or brain tissue, or can be made synthetically.

A preferred anionic phospholipid of the BL type (but close to being a ML-amphipat) is phosphatidylglycerol (R⁴ in Formula (IV) is glycerol). Another anionic phospholipid of BL type (close to being an IM-amphipat) is phosphatidic acid (R⁴ in Formula (IV) is a proton). To eliminate pH sensitivity of the latter phospholipid in a neutral pH range, R⁴ may be chosen to be a short-chain alkyl, such as methyl or ethyl. Phosphate- or sulphate-diesters with two similar or dissimilar, linear or branched, saturated or (poly)unsaturated, sufficiently long aliphatic chains covalently attached to the sulphate/phosphate group are synthetic analogues to phosphatidic acid, as is a not too charged AOT, or docusate.

Suitable sulphate-esters of dialkyl sulphosuccinate type are generally described with the Formula:

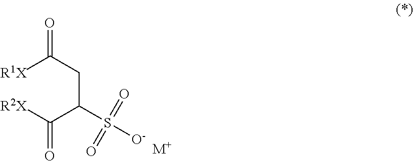

wherein R¹, R² independently of one another and identically or differently are H, an unsubstituted or substituted C1-C30 hydrocarbon radical, such as C1-C30 alkyl, or a (poly)alkylene oxide adduct, M⁺ is a cation, and X, Y are independently of one another identical or different and either O or R⁴N (or R³R⁴N+ or R⁴HN+). R⁴ is hydrogen, an unsubstituted or substituted C1-C30 hydrocarbon radical, such as C1-C30 alkyl, C1-C30 alkyl-C6-C14 aryl or poly(C6-C14-aryl-C1-C30-alkyl)phenyl, dicarboxyethyl or a (poly)alkylene oxide adduct.

Natural lipids having a net positive charge are rare. More suitable for the present invention are artificial cationic lipids, which must normally carry at least two hydrophobic segments to be of BL-type. The corresponding single-chain derivatives, with exception of those with many C-atoms per chain, are typically of the ML-type. The positively charged group normally contains a nitrogen atom, typically in the form of an ionised amino-group, but can comprise an -onium cation as well. N-fatty-residue-1,1′-iminobis-2-propanol, as in N-oleyl-1,1′-iminobis-2-propanol is another option. The hydrophobic residue is ideally a preferred chain as defined herein.

A useful example for the permanently cationic lipids based on the quaternary phosphonium compounds has the general Formula:

[R¹(R²)₂P⁺R³]X⁻

wherein R¹ is a proton, a C1-C6 alkyl radical, a C1-C6 hydroxyalkyl radical, or a C1-C6 aryl radical. In ML-class amphipats of this type, R² is a proton, a C1-C6 alkyl radical, or a C1-C6 hydroxyalkyl radical. R² radical extension to a C8-C18 alkyl, aryl, or heteroaryl radical gives a BL class amphipat. R³ is in either case a C8-C18 alkyl, aryl, or heteroaryl radical. X⁻ is typically a halide atom, but can also be another anion kind. A sufficiently hydrophobic molecule of this type can act as a microbicide. Similar formulas pertain to sulphonium cation as well, in like fashion.

Notwithstanding the tendency of many single chain amphiphiles to separate from the suspending medium, and then to form bi-, tri- or even multi-phasic systems, some such amphipats are practically useful adaptable aggregate forming entities. They can be held together by a hydrophobic interaction and hydrogen bonds, for example, and in some situations be supported by electrostatic interactions. This is true for, e.g., about stoichiometric fatty acid/fatty soap or fatty alcohol/fatty soap mixtures. An optimal pH range to prepare bilayers from such two-component fatty acid/fatty soap mixtures is therefore in the range from about 7 to about 9.5, preferably in the range from about 7.5 to about 8.5 (Walde et al., op. cit.), whereby longer chains typically require somewhat higher preferred pH values. Further examples of single-chain surfactant pairs that belong to BL-class, and thus spontaneously form fluid and sufficiently flexible bilayers, are about stoichiometric fatty-acid or -alcohol/lysophospholipid mixtures. Such fatty-acid or -alcohol/lysophospholipid combinations can involve similar or different types of chains.

Surfactants suitable for making and using preparations of the invention are also compounds from the polysaccharide betainate family of the following Formula:

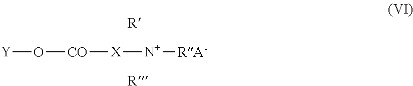

wherein R′, R″, and R′″ may be identical or different and are either linear or branched, saturated or unsaturated C1-C6 hydrocarbon radicals optionally interrupted by at least one heteroatom (chosen from nitrogen, oxygen, or sulphur) or else optionally substituted with at least one entity being either —OH, a halide (such as chlorine, bromine and iodine) or a C6-C24 aryl radical. X is a linear or branched, saturated or unsaturated divalent C1-C30 hydrocarbon radical, optionally interrupted by at least one hetero-atom chosen from nitrogen, oxygen, or sulphur, and optionally substituted with at least one hydroxyl radical; A⁻ is an anion and Y is a polysaccharide residue. Excluding compounds of Formula (VI) in which Y represents a polymeric starch structure, X is —CH₂—, and R′=R″=R″ is a methyl. For example, the identical or different R′, R″, and R′″ may be linear or branched, saturated C1-C6 hydrocarbon radicals, such as C2-C4 radicals, or a methyl radical.

In one embodiment, R′, R″, and R′″ are identical and, e.g., can be chosen from linear or branched, saturated C1-C6 hydrocarbon radicals, such as a methyl radical. In another embodiment, X is a linear or branched, saturated, divalent C1-C4 hydrocarbon radical, such as methylene, ethylene, propylene or butylene.

Also useful in the invention are the imidazoline-derived amphoteric surfactants. Most of these compounds can be described as fatty acid/amino-ethylethanolamine condensates with the following general structure:

R—CO—O—CH₂CH₂—NH—CH₂CH₂—NR′R″

wherein R is a fatty acid residue and R′ and R″ can be any of the functional units described previously (and the free tertiary amine can be alkylated to produce a quaternary ammonium compound with a permanent positive charge). The four main classes of the resulting compounds are: (i) amine/carboxylic acids containing both free amine (—NR₂) and free acid (—COOH) functionalities; (ii) quaternary ammonium/carboxylic acids, which contain a permanent cationic site (—N⁺R₃) and the carboxyl group; (iii) amine/sulphonic acids (or sulphate esters), which form internal salts and are essentially isoelectric in very acidic media; (iv): quaternary ammonium/sulphonic acids (or sulphate esters) and the highly ionizing strong acids.

A useful and unique form of the ring-opened imidazoline-surfactants are the so-called betaines, with alkyl- and alkylamidopropyl-betaines as the most universally used subtypes. One typical formula for a betaine is:

Y—(CH₃)₂—N⁺CH₂—R

wherein R can be a carboxy- or sulpho-group, Y is a C6-C30 aliphatic chain. (A molecule with C₁₂ and R=COOH can thus be called dodecylbetaine or N-dodecyl-N,N-dimethylglycine or dimethyldodecylammonioacetate or ethanoate; a molecule with C18:1 N-octadecanoyl-N,N-dimethylglycine or N-oleyl-N,N,dimethylglycines.)

Amphoteric alkyl amine oxides are potentially useful for the invention too. They can turn into cationic surfactants after amino-group protonation near and below their pK, as can the other related amphipats (including alkamidoalkylamine oxide (e.g. alkylamidopropylamine oxide, including but not limited to lauryl, myristyl-, palmityl, and oleyl-amidopropylamine oxide) Dimethyl (2-hydroxy-3-sulfopropyl)-acylammonium hydroxide (hydroxysultaine) represents another interesting amphoteric surfactant. Polyethoxylated amides are also useful for the invention.

Additional examples of the useful amphoteric lipids include aliphatic or aromatic derivatives of imino acids, which contain carboxylic and imino groups. Related entities include multi-ionic alkyl ethylenediaminetriacetate; the alkyl residue is a preferred chain.

A unique class of polymeric surfactants are POE-POP block copolymers, having the generic name “poloxamer” and the general formula:

HO(C₂H₄0)ₐ(C₃H₆0)_{b}(C₂H₄0)ₙH

wherein “a” hydrophilic POE and “b” hydrophobic POP segments are combined in certain ratios and positions to generate a variety of surfactants useful for the invention.

Relatively more polar components, and thus normally of the ML-type, are the partially or completely ionised monocarboxylic acid esters, such as alkyl-lactate, dicarboxylic acid esters, such as alkyl succinate, tricarboxylic acid (di)esters, such as (di)alkyl citrate, and tetracarboxylic acid (di)esters of (preferred) chains. Further useful esters are derived from the C8-C24 dicarboxylic acids and C8-C24 alcohols, from C8-C22 tricarboxylic acids and C8-C22 alcohols, higher degrees of polyacid ionisation requiring higher C-number for the formulations of the invention; furthermore, esters derived from mono-, di-, and tricarboxylic acids and alcohols chosen from C7-C26 di-, tri-, tetra- and pentahydroxy alcohols. Representative acyl-alkyl citrates of the invention include, but are not limited to, at least one alkyl ether citrate chosen from monoesters and diesters formed from citric acid and at least one C8-C30 oxyethylenated fatty alcohol. When used, the alkyl ether citrates can be neutralised with suitable simple or complex, inorganic or organic salts.

The alkenyl succinates useful for the invention include, but are not limited to, alkoxylated alkenyl succinates, alkoxylated glucose alkenyl succinates, and alkoxylated methylglucose alkenyl succinates of the following Formulae:

HOOC—CHR—CH₂—COO-Eₙ (VII)

HOOC—CHR—CH₂—COO-Eₙ-O—CO—CH₂—CHR′—COOH (VIII)

wherein R and R′ may be identical or different and are each chosen from linear or branched alkenyl C6 to C24 radicals. The number n of oxyethylene or oxypropylene units (in either case “E”) ranges from around 2 to around 100. In a random and block copolymer Eₙ is comprised of oxyethylene chains of formula (C₂H₄O)ₙ and oxypropylene chains of formula (C₃H₆O)_{n′} (such as oxyethylenated glucose copolymers, oxyethyleneatedmethylglucose copolymers, oxypropylenated glucose copolymers, and oxypropylenated methylglucose copolymers) such that the sum of n and n′ ranges from about 2 to about 100 and more preferably from about 4 to about 20, the oxyethylenated and oxypropylenated glucose groups of said oxyethylenated and oxypropylenated glucose copolymers have on average from about 2 to about 100 units and more preferably from about 4 to about 20 units, respectively, oxyethylene or oxypropylene units distributed on all hydroxyl groups, and the oxyethylenated and oxypropylenated methylglucose groups of said oxyethylenated and oxypropylenated methyl glucose copolymers have on average from about 2 to about 100 oxyethylene or oxypropylene units and more preferably from about 4 to about 20 units distributed on all hydroxyl groups. Hydrophobic chains can act as preferred chains as defined herein.

Suitable anionic amphiphilic amphipats of the invention are also alkyl and alkoxylated glucose alkenyl succinates, and alkoxylated methylglucose alkenyl succinates.

In addition to anionic carboxylates, one can advantageously use alkyl- or alkenoyl-organic group salts (such preferred chain-phosphate, phosphonate, or phosphinate salts, or else the corresponding alkyl aryl ether phosphate and alkyl ether phosphate, phosphonate or phosphinate salts. The related-sulphate or sulphonate, as well as the corresponding alkyl aryl sulphonates salts are useful for the invention too.

Some of the suitable alkylsulphonic- or phosphonic derivatives are described by the formula:

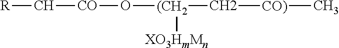

wherein R is a C6-C24 alkyl chain, M is a suitable salt, which can be a preferred ion, m is 0 or 1, n is 1 or 2, and X is either a sulphur or a phosphorous atom. Further non-limiting examples of sulphonates or phosphonates include 3-(long fatty chain-dimethylammonio)-alkane-sulphonates or -phosphonates, e.g. 3-(acyldimethylammonio)-alkanesulphonates, the long fatty chain derivatives of sulphosuccinates described with the general formula (*) and the sulpho- and phosphor-mono or diesters, mentioned elsewhere in the text, with around 8 to around 40 C-atoms in total.

Yet another interesting group of ionic sulphonic amphiphiles, including several BL class lipids, are alkylbenzene sulphonates. A particularly well known representative of BL-class is dodecylbenzene sulphonate, but other alkyl lengths are useful for the invention too. Further useful ionic surfactants include the dissociated salts of gall-acids including but not limited to simple or complex, organic or inorganic salts of cholate, deoxycholate, glycocholate, glycodeoxycholate, taurodeoxycholate, and taurocholate.

The long-chain quaternary ammonium salts, fatty amines, and salts thereof are useful for the invention as cationic lipids in addition to those defined herein. The former group includes, but is not limited to, the single fatty-chain ammonium salts, such as alkyl- or alkenoyl-trimethyl-, -dimethyl- and -methyl-ammonium salts, fatty chain dimethyl-aminoxides, such as alkyl-, alkenoyl-, or alkanoyl dimethyl-aminoxides, fatty chain, e.g., alkyl-, alkenoyl-, or alkanoyl-N-methylglucamides, N-long fatty chain-N,N-dimethylglycines, e.g., N-alkyl-N,N-dimethylglycines, which are normally of ML-type for not too long fatty chains.

A quaternary ammonium salt can have the general Formula:

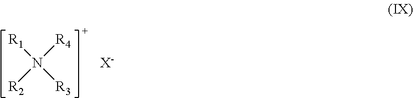

wherein R₁, R₂, R₃, and R₄ may be identical or different and are either aliphatic groups comprising from 1 to 30 C-atoms and/or aromatic groups, such as aryl and alkylaryl groups. The aliphatic groups can comprise hetero atoms, e.g., oxygen, nitrogen, and sulphur. The aliphatic groups can be chosen, e.g., from alkyl, alkoxy, polyoxy(C2-C6)-alkylene, alkylamide, (C12-C24)-alkylamido(C2-C8)-alkyl, (C12-C24)-alkylacetate, and hydroxyalkyl groups with from 1 to about 30 C-atoms. X⁻ is an anion.

Also suitable for the invention is a quaternary ammonium salt of imidazolinium, e.g., a salt described by the Formula:

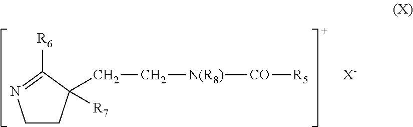

wherein R₅ is a C1-C4alkyl group and R₆ is a hydrogen atom or a C1-C4alkyl group. In one embodiment, e.g., R₅ and R₆ are chosen from alkenyl and alkyl groups with from about 12 to about 21 C-atoms, e.g., alkenyl and alkyl groups derived from a suitable oil as defined herein, and wherein said R₅ and R₆ are chosen such that said quaternary ammonium salts of imidazolinium comprise at least one alkenyl group and at least one alkyl group, R₇ being methyl, and R₈=H. X⁻ is a suitable anion.

The quaternary ammonium salt can moreover be, e.g., a diquaternary ammonium salt of Formula:

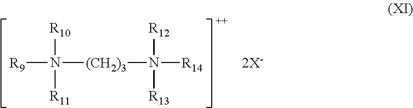

wherein R₉ is chosen from aliphatic groups with about 16 to 30 C-atoms. R₁₀, R₁₁; R₁₂, R₁₃ and R₁₄, which may be identical or different, are each chosen from H-atom and alkyl groups with 1 to 4 C-atoms, and X⁻ is a suitable anion.

The quaternary ammonium salt can also include at least one ester function having the general Formula:

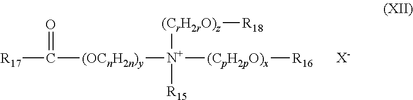

wherein R₁₅ is a C1-C6 alkyl group, a C1-C6 hydroxyalkyl group or a C1-C6 dihydroxyalkyl group, and R₁₆ is an acyl group of the following Formula:

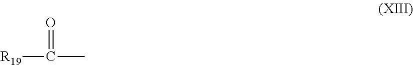

wherein R¹⁹ is an aliphatic chain, or a hydrogen atom, and R₁₈ is an acyl group of the following Formula:

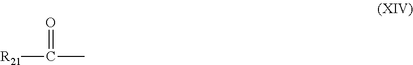

wherein R₂₁ is an aliphatic chain or a hydrogen atom. R₁₇, R₁₉ and R₂₁ of Formula (XII) may be identical or different and are each an aliphatic C7-C21 chain; n, p and r may be identical or different and are integers with values between 2 and 6; y is an integer with a value between 1 and 10, and x and z, which may also be identical or different, are also integers ranging from 0 to 10. R₁₆ is a C7-C24 aliphatic chain whenever 1<x+y+z<15 and x=0. When z=0, then R₁₈ is a C1-C6aliphatic chain. The sum x+y+z can range, e.g., from 1 to 10. When R₁₆ is a C1-C24aliphatic chain, R₁₆ can be long and have from about 12 to about 24 C-atoms, or short and have 1 to 3 C-atoms. When R₁₈ is a C1-C6aliphatic chain, R₁₈ can have from 1 to 3 C-atoms. The R₁₅ alkyl group may also be linear or branched; e.g., R₁₅ may be linear and from the group including a methyl, an ethyl, a hydroxyethyl or dihydroxypropyl group, with some preference for methyl and ethyl groups. In Formula (XII), X⁻ denotes an anion. R₁₇, R₁₉ and R₂₁ may be identical or different and each an aliphatic C11-C21 chain, e.g., x and z may be identical or different and can each take value of 0 or 1; y, e.g., may be equal to 1. n, p and r, which may be identical or different, can, e.g., each have the value of 2 or 3 and in one embodiment are both equal to 2. Further exemplary ammonium salts of Formula (XII) are those in which R₁₅ is a methyl or an ethyl group, x=1, y=1, z=0 or z=1, and n, p, r are all equal to 2. R₁₈ is, e.g., an acyl group of the Formula (XIII) wherein R₂₁=H. R₁₇, R₁₉ and R₂₁ may be identical or different C13-C17 aliphatic chains, e.g. a linear or branched, saturated or unsaturated C13-C17 alkyl and alkenyl, linear or branched chains. When the compound is comprised of several acyl groups, such independently selectable groups may be identical or different.

It is also possible to use the ammonium salts with at least one ester function, or at least one hexosamine in the present formulations.

Additional ionic surfactants suitable for use in the invention are salts of acylated amino acids and their derivatives, including salts of C6-C22 acylated amino acids, e.g., the preferred chain sarcosinates.

### 5.2. Compositions

The present invention relates to certain amphipat, or surfactant, ratios that are considered when developing the formulations of the invention. Said ratios can be expressed as mol-per-mol (or mol/mol or mol:mol) or as weight-per-weight ratios. In the ratio calculation, each compound associated with an aggregate bilayer is accounted for. The most elementary embodiments according to the invention concern aggregates formed from at least one commercial surfactant with a sufficiently broad distribution of molecular species to allow partial localized molecular ‘demixing’ supporting aggregate deformation. (Note that increasing molecular weight/size/headgroup and potentially tail-lengths distribution, or difference, affects the effective Ac or nP or HLB, and typically results in a relatively higher final effective Ac or nP or HLB requirement.)

In other exemplary but non-limiting embodiments, the chosen composition includes two kind of molecules, one from BL class (or MFC, previously described) and another from the ML class (or MDC, previously described); molecular distribution width again plays a role (as is evident from the higher adaptability of the aggregates of Example 36 vs. Example 32 herein). The former molecule can have two hydrophobic tails, e.g., and the second then typically has one such tail. The first and often more abundant amphipat, correspondingly, has a lower area per chain and a lower polarity units number than the second, less abundant amphipat. The preferred mixture of these amphipats is such that the weighted sum of Ac and/or polarity units number and/or HLB also corresponds to BL-class, but is close to its upper limit. In several embodiments, the targeted area per fluid chain with 18 C-atoms (e.g. C18:1) is therefore in the range Ac˜0.43-0.47 nm², on the average. The calculated target Ac value for C12 may be around 10-20% lower. Correspondingly, the targeted final combined HLB number should be between 6.5-7.5 and 13.5-12.5, more preferably between around 8 and around 13 and most preferably around 10.5±2.5. This can, but need not, yield the 1^{st} and the 2^{nd} amphipat molar ratio from about 20:1 to about 1:10, and more often in the range from about 5:1 to about 1:3. The preferred molar ratio decreases, i.e. more of the second amphipat is needed, if the first amphipat Ac and/or HLB number is closer to the lower BL-class criterion limit, and vice versa.

For amphipats with polymeric heads one can specify the preferred repetitive units number per hydrophobic chain as well. For fluid-chain polyoxyethylene-fatty ethers, e.g., the preferred repetitive units number per hydrocarbon chain is between around 5nC/24 and around 8.5nC/24. Such preferred at least one first amphipat in the formulations of the invention can optionally be supplemented with a second amphipat having a similar or different, but typically more polar (i.e. for the similar structures longer) headgroup(s); the repetitive units number in the first amphipat should then be lowered to maintain the overall polarity units number in the specified range, or only moderately above; the tolerable excess increases with the chosen headgroups length difference and with fatty-chains length. To select different preferable headgroups, the “polarity unit” concept introduced herein is useful. In any case, a polarity unit value near the upper specified limit yields practically more effective formulations than those polarity unit values nearer to the lower specified limit. It is noteworthy some vesicles that form quasi-spontaneously, i.e. with essentially zero vesicularisation time as defined herein, can become unstable upon storage.

When more than two amphipats are combined in one formulation, the given ranges apply, very broadly, to the ratio of the more lipophilic surfactants grouped together (with a lover average HLB value) and of the more hydrophilic surfactants grouped together (with a higher average HLB value).

In some exemplary but non-limiting embodiments of the invention the ratio for the blends of relatively different amphipats (Ac_surfactant>>Ac_lipid) ranges from about 1:1 to about 2:1, from about 2:1 to about 3:1, from about 3:1 to about 4:1, from about 4:1 to about 5:1 or from about 5:1 to about 10:1. In some specific embodiments, the lipid to surfactant ratio is about 1:1, about 1.25:1, about 1.5:1, about 1.75:1, about 2:1, about 2.5:1, about 3:1, about 4:1 or about 5:1. When both amphipats are relatively similar (Ac_surfactant˜Ac_lipid), the lipid to surfactant ratio is often about 1:1, about 1:1.25, about 1:1.5, about 1:1.75, about 1:2, about 1:2.5, about 1:3, about 1:3.5 or about 1:4. When one amphipat is a phospholipid, its molar ratio to the second amphipat may be about 1:1.25, about 1:1.5, about 1:2, about 1:2.5, or even higher.

In those embodiments comprising at least one amphipat with more than two aliphatic chains, such amphipat is typically characterised by a low area per chain and a relatively low polarity units and/or HLB number. A relative high concentration of the amphipat with higher Ac value/polarity units/HLB number (the surfactant proper or surfactantsgroup) may then be necessary to ensure aggregate functionality according to the invention. Ideally, relative concentration of the surfactant(s) with a relatively high polarity units/HLB number in a multi-component blend should be low, normally<about 30 rel. mol-%, preferably<about 20 rel. mol-% and even more preferably≦10 rel. mol-%. The same applies to the surfactants characterised by relatively low polarity or HLB numbers, especially to oils, unless such components are specifically desired.

In the embodiments comprising several amphipats held together by ionic and/or hydrogen bonds, the paired-components are preferentially used in about stoichiometric ratio, i.e. in the molar ratio about 1:1 for monovalent surfactants, and 2:1 or 1:2 for the mono- and divalent amphipats combinations, respectively. Examples 115 and 116 exemplify such non-limiting technological solutions.

The at least one vesicle stabilising amphiphilic lipid that is selected from nonionic amphiphilic lipids may be included into the preparations of the invention in a range from 0.1% to 30% by weight relative to the total weight of the preparation, e.g., especially from about 0.5% 1% to about 20% and preferably from about 5% to about 10% by weight relative to the total weight of the preparation. The at least one vesicle stabilising amphipat can moreover be chosen from either BL-type cationic amphipats or anionic amphipats, other than the anionic amphipats described above. Practically useful examples include but are not limited to, the salts of diacyl phosphate or its lower alkyl monoester, phosphonate, sulphate or sulphonate, especially if attached to similar or dissimilar preferred chains; salts of cholesteryl phosphate or sulphate; long fatty soap or amino acid salts, such as monosodium and disodium acyl-glutamate or -sarcosinate, for instance the mono- or disodium salt of N-oleoyl-L-glutamic acid or phosphatidylglycerol. Such additive concentrations range from about 0.01% to about 50% by weight relative to the total mass of all amphipats in the formulation, more often from about 0.5% to about 35% by such relative weight, and more preferably from about 1% to about 25% such relative weight.

Some embodiments are chosen to contain between 0.1 wt.-% and up to 50 wt.-% of combined amphipats mass; more typical concentrations range between about 0.5 wt.-% and about 25 wt.-% and even more preferably between about 1 wt.-% and about 15 wt.-%. The combined amphipats quantity is preferably lower for cutaneous indications (typically up to 15 wt.-%) than for deep tissue indications (typically above about 1 wt.-%). Again, the rules of establishing and subdividing ranges apply as defined herein.

Any formulation of the invention may optionally contain antimicrobials and other preservatives, antioxidants, chelators, co-solvents (such as short-chain, i.e. lower alkyl alcohols), emollients/humectants (such as glycerol), enzyme inhibitors, fragrances and even flavours, as well as thickeners, either each of them alone or in any suitable and pharmaceutically acceptable combination. Inclusion of antimicrobials is often mandatory, unless single-use primary packaging material is used. The typical concentration range is in the range from about 0.05 wt.-% to about 5 wt.-% relative to total surfactant mass that is typically about 10 wt.-%. Where possible, no antioxidant and/or chelator is included, to minimise the number of components. If an additive is needed, it is preferably hydrophilic. If it must be lipophilic, its total concentration should ideally be in the range up to 10 wt.-%, and more preferred up to about 5 wt.-%, relative to total amphipat mass in the formulation. Ideally, no additive relative concentration should exceed 5 wt.-% of the collective amphipats concentration. The hydrophilic antioxidants concentration is often used in similar preferred range of total weight concentrations.

Any formulation according to this invention may contain a fragrance, to increase the appeal of the final preparation, improve patient compliance and/or mask the natural odor of the composition components. Fragrance concentration should be low but sufficient, since fragrance partitioning into mixed amphipat aggregates can diminish a desirable olfactory effect. In some embodiments of the invention, the fragrance concentration ranges between about 0.1% and about 5% and more preferably between 0.5% and 2.5% by weight relative to the combined weight of amphipats.

If buffering capacity of the employed amphipats or thickeners is insufficient to keep formulation pH-stable and near the desired value, a buffer should be included into a preparation to adjust and/or to maintain the preparation pH constant. Unless specified otherwise, the bulk pH is typically chosen in the range from about pH=2.5 to about pH=9.5, from about pH=3 to about pH=8.5, or from about pH=4 to about pH=7.5. Neutral formulations have preferably values about 6.5, cationic formulations a lower pH value and anionic formulations a higher pH value, the difference increasing with increasing charge density on the mixed amphipat bilayers. Preparations for use on skin may be more acidic, to match the skin surface pH, which is normally around pH=5±1.

Methods for adjusting the required buffer concentration to the given formulation needs are generally known. Useful buffers include but are not limited to acetate, lactate, phosphate, sulphate, and propionate and are normally selected depending on the desired final pH value. The added buffer concentration is typically in the 5-250 mM range, preferably from about 15 mM to about 150 mM and preferably not higher than 50 mM.

The suspending medium of the formulations is typically an aqueous solution, which advantageously permits the composition to be a suspension or dispersion, which may be sprayable. The preparations of the invention may additionally contain excipients useful for the spraying process, or subsequent distribution of the formulation over the site of application. Formulations of the invention can also be incorporated into a suitable emulsion, cream, lotion, ointment, gel, or a film forming solution, as desired. Formulations may have to be adjusted to optimize the therapeutic effect, especially if said emulsion, cream, lotion, or ointment presents a large surface area and/or contains a significant proportion of dissolved amphipats or ions.

To form a hydrophilic gel a thickener may have to be included into a formulation, typically in a concentration range from about 0.25 wt.-% to about 5 wt.-% relative to total preparation weight; more preferably a thickener is employed in the range from about 0.5 wt.-% to about 2.5 wt.-%, as is necessary to increase the viscosity of the aggregate preparations to between around 0.05 Pa s and around 10 Pa s, preferably between around 0.15 Pa s and around 5 Pa s, and most preferred between around 0.3 Pa s and around 2.5 Pa s. The generally preferred types and amounts of the different ingredients that can act as optional thickeners, unless reported specifically herein, are known. They may have to be adjusted moderately in the embodiments of this invention to compensate the viscosity-modifying effects of the inventive aggregates or their components, if any. For example, the relatively lipophilic additives concentration may have to be modified relative to that useful in an essentially aqueous preparation, to compensate for such additives association with /binding to the bilayers.

Some embodiments may be also comprised of at least one co-solvent. When included in a preparation, the at least one co-solvent concentration then generally ranges, e.g., from about 0.01 wt.-% to about 30 wt.-%, relative to the total weight of the preparation. If the at least one solvent is a mono- or diol, with predominantly polar character, e.g., ethanol, propanol, propane-diol, etc., its concentration is often chosen in the range from about 1 wt-% to about 15 wt.-% and preferably below 10 wt.-% and most preferably below 5 wt.-%. When the formulation contains such an alcohol in the relative weight concentration of at least 5% and more often of at least 10% to 15% by weight relative to total preparation weight, the product may require no additional antimicrobial agent. If the at least one solvent is glycol or polyethylene glycol, its concentration is advantageously in the range from about 1 wt.-% to about 30 wt.-% and preferably between around 5 wt.-% and around 10 wt.-%.

Use of a suitable acid, such as sorbic acid, benzoic acid, acetic acid, formic acid, or propionic acid, e.g. (antimicrobials, defined above) as a buffer at a sufficiently high free-substance concentration may eliminate the need to add further antimicrobials to the preparation. Other alternative components of the present formulations such as anti-oxidants or surfactants can be selected and used at concentrations that effectively eliminate microbial action, optimally at a pH 5 (see e.g. W. Paulus, ed. op. cit.).

Typical aggregates of the invention can be microscopic, i.e., up to a 5 μm large, but preferably are sub-microscopic, i.e., have an average diameter of between 20 nm and 750 nm. (A preferred range is, e.g., from about 25 nm to about 250 nm, and even more preferably is from about 30 nm to about 200 nm.) To quantify the average aggregate size, one can analyse, e.g., the dynamic light scattering on a preparation using a photon-correlation spectrometer (e.g. a Zetasizer® or Autosizer®, Malvern). Alternatively, a UV/vis spectrophotometer can be used, e.g. by analysing the turbidity- or the wavelength-exponent-spectrum, as described by Elsayed & Cevc (op. cit.).

### 5.3 Aggregate Preparations

Depending on the desired composition, the prevailing physical properties and presentation form, the components suitable for making compositions of the invention can be solid, waxy or fluid. To ensure proper mixing, all components should preferably be in a liquid form prior to combining them. This step may be done separately for the lipophilic/amphipathic components and the water-soluble components.

### 5.3.1 Preparation of an Aqueous Mixture

A person skilled in the art will know how and in which sequence to mix water-soluble components in an acceptable, typically aqueous, medium for subsequent fluid or dissolved amphipats blending-in. A judicious choice of pH, salt(s) kind/concentration, and temperature, will promote fast and complete solubilisation of all hydrophilic components in such medium. Visual homogeneity checks normally suffice for monitoring the dissolution. pH measurements provide additional insight. Preparation thickeners, if any, are preferably introduced as late as possible during the manufacturing process.

### 5.3.2 Preparation of an Organic Mixture

Unless all the chosen amphiphiles are fluid, or completely liquefied through heating, it is prudent to dissolve as many lipophilic components as possible in the prevailing fluid organic component of the formulation (e.g. polysorbate 80, Brij 98, an unsaturated fatty acid, a mixture of phospholipid and a co-solvents, or the like). One useful temperature range is from about 5° C. to about 95° C., preferably from about 15° C. to about 60° C. and even more preferably from about 20° C. to about 45° C. Exceptions are amphipats with a low cloud point, which are better processed at low temperatures.

Solubilisation of all non-fluid lipophilic compounds in fluid lipophilic components may be assisted by pharmaceutically acceptable co-solvents (preferably glycerol, ethanol, propanol, or iso-propanol). Concentration of co-solvent used just for the purpose should be as low as possible, but typically in the range from about 1 wt.-% to about 80 wt.-%, preferred from about 2 wt.-% to about 50 wt.-%, and more preferred from about 3 wt.-% to about 25 wt.-%. Vessel agitation supports the mixing. To facilitate uniform amphipat hydration during the next step, the solubilised amphipats should be brought into contact with the suspending medium more or less instantaneously, and at least in a controlled fashion. One can introduce the organic mixture into the aqueous medium gradually, e.g., by dripping, injecting, or drawing the former into the latter.

Carefully chosen rate of organic mixture addition to the well stirred aqueous mixture can improve the formation of acceptably small and/or uniformly sized aggregates. Stirring devices supporting the process include but are not limited to simple mixers, blade mixers, flow-through (i.e. in-line) mixers, and homogenisers (such as high-shear, e.g. rotor-stator devices, or high-pressure devices). A preferred method for introducing one mixture into the other is the injection of one (e.g. organic) mixture through sufficiently fine nozzles into the other (e.g. aqueous) bulk. If pouring is used instead, the stream of added (organic) mixture should not be too thick/too fast. Another preferred method is drawing one (e.g. organic mixture) into the other (e.g. aqueous) mixture through an inlet under reduced pressure. Powders can be added in like manner.

If the average aggregate/vesicle size of the resulting formulation does not meet the desired specifications after the mixing, then the crude dispersion can be homogenised further by exerting sufficiently high-stress on it, frequently using a high-shear mixer or extrusion through a (set of) porous filter(s) in a convenient holder.

### 5.3.3 Functional Testing of the Preparations of the Invention

In Vivo Testing.

The precise mechanism(s) of action underlying the invention is still open to speculation, but arguably involves physical effects. The most definitive and conclusive support of the invention are thus biological assays conducted in vivo in mammals, especially humans. Many provoked-inflammation type tests are known. They normally assess and quantify anti-inflammatory effects visually, and occasionally evaluate an anti-pain effect in parallel. Mustard-oil induced inflammation of human skin has been used to test preparations of the invention, due to the diversity of the underlying reactions. (For preclinical characterisation of mustard-oil in a murine model see: Inoue et al., 1997 Eur J Pharmacol 333: 231. One model used to test the compositions of this invention in humans is published (Cevc, 2012, J Contr Rel, see http://dx.doi.org/10.1016/j.jconrel.2012.01.005). In brief, such compositions and reference products were pre- or post-applied on the skin challenged with an individually standardised amount of mustard oil. The resulting suppression of skin redness (erythema) and swelling (edema) was observed and recorded over time. Any difference between a cumulative effect of the various treatments and non-treatment was assessed to determine the relative therapeutic efficiency of the tested preparations.

In Vitro Testing:

Each tested formulation was checked for the following: aggregate suspension homogeneity; the homogeneous preparations, if any, were then subjected to aggregates adaptability test (normally the described sonication test and occasionally to pore penetration test); adaptable aggregates size in suspension (by checking its optical density either in absolute terms with a UV/vis spectrophotometer or in relative terms by comparison with an equally concentrated “standard” preparation using a similar optical path); suspension stability (by confirming that the tested preparation retained its essential characteristics during storing). Moreover, the water retention ability of each formulation tested in vivo was assessed by haptically monitoring the formulation drying on an open skin or a clean organic surface.

### 5.3.4 Skin or Deep Tissue Pain Treatments

To affect subcutaneous tissue, even if only indirectly, the epicutaneously deposited quantity of the formulations according to this invention should be in the range from about 0.5 mg amphipat per cm² to about 2 mg amphipat per cm² and preferably around 1 mg cm⁻², distributed uniformly (without the need for rubbing). For more superficial tissue treatment a smaller material quantity suffices, but should be ≧50 μg cm⁻².

### 6. EXAMPLES

### 6.1 Preparations without a Net Charge

Table 1 provides over one hundred representative, but not limiting, examples relying on at least one cyclic hydrophobic (Examples 1, 2) or a linear aliphatic chain attached via an ether or ester bond (Examples 3-22, commercially available Brij® (Uniqema), Emalex® (Nihon Emulsion), Emulsogen® LP (Clariant), etc.) or through an amide bond directly to at least one polar headgroup (Example 23; commercially available Ethomid®, Akzo Nobel). The latter compound in the first group of embodiments typically comprises a PEG chain, i.e. is a chain of ethyleneglycol (“EG”) units.

### Examples 1, 2

Highly adaptable bilayer vesicles can be manufactured from nonionic surfactants of the aryl-type without further additives. Commercial examples (octyl-: Triton® (Dow), Macol® (BASF), Igepal CA® (Rhodia), etc.; nonyl-: Tergitol® (Dow), Hostapal®, (Clariant) Igepal COO, Trycol® (BASF), etc.) for nominally similar molecules are usually broadly interchangeable; Table 1 therefore specifies in columns 11-14 (identified through headings “Head 1” to “Head 4”) the nominal average number of EG units per molecule rather than using specific tradenames, or EG/n₁. Chain-length is specified in column 2=“L1′”, since all amphipats used in the compositions of this group have similar nominal chain length, unless specified otherwise in column 6. The corresponding number of double bonds is indicated in the 3^{rd} or 7^{th} column (i.e. “DBₓ”, where x identifies the sequential number of the used amphipat). The average number of hydrophobic chains per amphipat is specified in the 4^{th} to 6^{th} (=“nₓ”) column. The three columns headed “Bond type” (columns 8-10) in Table 1 identify the 1^{st} to 2^{nd} (and, possible 3^{rd}) amphipat-bond type. Columns 15-17 define individual formulation compositions, expressed either in terms of weight concentrations (w+w, column 15), molar fractions (M+M, column 16, not shown) or molar ratio (M/M, column 17, not shown), where #x in the top-row gives the appropriate index for the entire column below: #x w≡wₓ and #x M≡Mₓ. Columns 23, 24 and 25 disclose the calculated Ac value, polarity units number (nP), and HLB number for each specific composition. nP is calculated from the known molar fractions (column 16) of single components with nEOx (cf. EG/nₓ in columns 11-14) A blend with M₁+M₂=0.75+0.25 and EG/n₁=2, EG/n₂=10 thus yields nEOeff=0.75×2+0.25×10=4.0 (see Example 10), as each EG corresponds to 1 polarity unit. Unless otherwise specified, the chosen electrolyte is 0.1 M sodium phosphate buffer with the pH given in column 19 and total amphipat concentration is 10 wt.-%. Illustrative Example 1 thus corresponds to a blend of two single-chain (columns 4, 5) octylphenol (columns 2, 3) ethoxylates with 3 and 7.5 EO units (columns 11, 12) ether-bonded (columns 8, 9) to phenol-ring (column 3). The first amphipat has molar mass 338 (column 19) and is used at concentration of 40 g L⁻¹ (left part of column 15) and the second amphipat has a molar mass 536 (column 20) and is used at concentration of 60 g L⁻¹ (right part of column 15), corresponding to respective molar fractions of 0.51 and 0.49 (column 16, not shown) giving a molar ratio of 2.38/1 (column 17, not shown). (CAVE:molar ratio “9999/1” in all tables means 1/0) The effective area per chain is 0.44 nm² (column 23), the effective number of polarity units pertaining to the blend is 0.7×3×+0.3×7.5=4.3 (column 24) and the corresponding HLB number is 9 (column 25). Unlabelled columns between 14 and 15 show exemplary commercial names for the amphipats used.

### Examples 3-23

Further illustrative Examples shown in Table 1 involve mainly PEG-fatty-ethers, and follow the same nomenclature as used in Examples 1 and 2. E.g. 10, e.g., the first amphipat, an octadecenyl-(═Oleyl)ether with nominally 2 EO units per headgroup #1 (e.g. Brij® 93 (Uniquema) or Volpo® N2 (Croda)) was used at an absolute concentration of 60 g L⁻¹. The second amphipat, an octadecenyl-ether with nominally 10 EO units per headgroup #2 (e.g. Brij® 97 or Emalex® 710 (Nihon) or Volpo® N10) was used at concentration of 40 g L⁻¹, corresponding to 0.75 and 0.25 respective molar fractions and to molar ratio of 2.97/1, giving the effective area per chain of 0.4 nm², the effective number of polarity units of 0.75×2+0.25×10=4 (as ether bonds require no additional correction), and HLB=6.8. The suspending medium was again phosphate buffer (100 mM) with a pH of about 7.4. In each group of related preparations, merely differing in molar ratio between the first and second amphipat, those preparations with the higher second amphipat relative concentration were found to contain more adaptable vesicular aggregates.

### Examples 24-44

Additional illustrative embodiments were prepared using commercial surfactants with several PEG chains and one (Examples 24-41, column 4) or more (Examples 42-44, column 4) hydrophobic chains attached via ester bonds to a common sorbitan ring (cf. columns 8, 9). In Example 41, e.g., an oleyl-sorbitan-ethoxylate with 5EO groups per head and chain, on the average (commercial examples: Tween® 81 or Tween® 82 (Croda) or Montanox® 81 (Seppic) was used as the first surfactant at a concentration of 50 g L⁻¹ (left part of column 15). An oleyl-sorbitan-ethoxylate with nEO˜20 per head and chain (commercial examples Tween® or Montanox® 80) in the embodiment as the second amphipat at a concentration of 50 g L⁻¹ (right part of column 15), giving molar fractions of 0.67+0.33 (column 16, not shown) and molar ratio 2/1 (column 17, not shown). The net effect is a heterogeneous amphipat bilayer with an average calculated polarity units count of 5.9=(0.67×5+0.33×20)/1.7 per fatty-chain, the average area per chain of 0.45 nm², and HLB=11.7.

Likewise, increasing the headgroup mismatch requires longer average headgroups to achieve the same degree of bilayer softening. Hydrophobic chain-length shortening has the opposite effect. (Preparations with the smallest Ac value, the lowest effective polarity units count and the lowest HLB number thus did not contain stable vesicle suspensions, but were rather ‘phase separated’ into at least one oil-rich (“microemulsion”) and one watery compartment, especially if aliphatic chains were relatively long. Intermediate Ac values, polarity unit counts, or HLB values (implying a too low concentration of bilayer-softeners) yielded relatively “stiff” vesicles. The preparations listed in each series with the highest relative concentration of the more polar amphipat contained the most adaptable aggregates (e.g. Examples 35, 40, 41, 43, 44). The notation used in Table 1 to describe the three-component Example 39 is explained below when discussing Table 2.

### Examples 45-59

Another illustrative group of embodiments involves polyglyceridic amphipats, which can nominally carry several hydrophobic “anchors” attached stochastically to the glyceride portion “G” (e.g. in Examples 48, 58, 59). Others have nominally a single fatty-chain (e.g. Examples 45-47, 49-57), but appear to contain a proportion of oligo-fatty derivatives as well. The resulting molecular polydispersity requires an a priori check of the actual molecular composition and/or an ad hoc determination of the effective polarity units number for each chosen polyglyceride brand. This notwithstanding, polyglyceride amphipats are valuable for making preparations of the invention, especially owing to their low sensitivity to temperature changes, biological origin, and mildness.

Table 1 specifies various compositions of the instant preparations made from relatively short and fully saturated (lauroyl-, see Examples 45, 46) or relatively long and (mono)unsaturated (oleoyl-, Examples 47-59) -polyglycerides (commercial example: Dermofeel® (Dr. Straetmans)). Example 45 specifies a preparation comprising lauroyl-pentaglyceride, nG=5, that forms micellar suspensions in an aqueous buffer. Example 46 pertains to a mixture of such polyglyceride with oleyl-alcohol, nG=0, yielding adaptable vesicles. In Example 47a pentaglyceride is coupled to nominally one, and in Example 48, to around 2 (calculated: 1.6) chains. In Example 49 the two amphipats are combined. Examples 50-56 relate to an oleoyl-diglyceride (nG=2, commercial example Emulsogen®) mixed in various proportions with a long-headed (nEO=20) oleyl-polysorbate ester. In Example 57, the former is combined with the amphipat of Example 47. The last two preparations in the group comprise a decaglyceride (commercial example Caprol® (Abitec)) coupled to around 1.5 oleyl-chains and used either alone (Example 58) or blended with nEO=20 oleoy-ethoxylated-polysorbate (Example 59). Experiments revealed that sufficient adaptability of the aggregates of the series depends on a relatively high molar concentration of the more polar chemically different amphipat (cf. Examples 56, 59), unless nG is close to the upper specified polarity unit limit. Mixtures with too low relative concentration of such second amphipat do not form stable bilayer vesicles, but instead ultimately gather in an oily upper phase (as in Examples 50-53).

### Examples 60-66

An additional, relatively temperature insensitive, group of surfactants has sugar headgroups, to which more than one hydrophobic chains can be, and often are, attached. Ester and amide bonds are most popular for the purpose. Table 1 lists several compositions employing relatively short chain (lauryl, Examples 60-64) or longer chain (oleyl, Examples 64-66) fatty residues attached to a mono-hexose (glucose, Example 60) or a disaccharide, saccharose (Examples 61-66). Example 60 contains a non-ethoxylated sorbate (lauroylsorbitane) as the less polar component. The series addresses effect of multiple hydrophobic anchors as well, which diminish relative potency of the sugar headgroup in comparison with the corresponding single-chain sugar surfactant.

### Examples 67-107

Compositions 71-80 and 84-91 all comprise a double-chain phosphatidylcholine having an Ac of about 0.33-0-35 nm² and thus resemble known formulations (which are useful as a control), leading to the highly adaptable vesicular preparations of Examples 77 and 87-89. Examples 78 and 90 are on the verge of being stable formulations and Examples 79, 80 and 90, 91 each contain an appreciable proportion of amphipats in an undesirable micellar form. Other Examples reflected in Table 1 expand on previously known highly adaptable vesicle suspensions, and involve either a combination of several non-synergetic bilayer-softening amphipats (Examples 81, 92-98, 100-103) plus an uncommon, typically synthetic, phosphatide (phosphatidyl-(N,N)-dimethylethanolamine, Examples 99-103) or the use of a single chain phosphatide (lyso-phosphatidylcholine, Examples 104-107), which does not spontaneously form bilayers and is therefore, from a stability vantage point, quite difficult to manage. An explanation of how to interpret the three-component Examples is provided in Table 2.

### 6.2 Formulations Comprising Charged Amphipats

Table 2 lists illustrative charged formulations, mostly derived from the preparations specified in Table 1. To demonstrate the broadly applicable nature of the invention, single- and double-chain, biological and synthetic amphipats are included.

### Examples 108-122

The first illustrative group of Examples in this section relates to adaptable aggregates of the compounds reported in Table 1, that are supplemented either with charged fatty-sulphate or -phosphate molecules of different hydrocarbon length and type. The first four Examples listed in Table 2 include an amphipat with saturated chains (the first two C12=lauryl and the second two C16=cetyl/hexadecyl) in addition to two original aggregate formers. The subsequent four Examples involve mono-unsaturated C18:1 chain(s) on all amphipats. Hydrophobic anchor attachment is either direct (as in Examples 109-113) or through a spacer, which may assume various shapes and have various compositions. The charge-spacer of Example 116 is cyclic and hydrophobic, e.g., and in Examples 108 and 114, the charge-spacer is linear and more hydrophilic (EG₆ or EG₅, respectively). Aggregates of the invention can be imparted a charge using fatty-amino-acids as well, e.g., by using a sarcosine headgroup (as in Example 115).

In detail, in Table 2, Example 108 is comprised of a blend of several single-chain (columns 4, 5, 8), ethoxylated (heading of the pertinent block in the table) lauryl (columns 2 and 6) and ethers (columns 9-11). To produce the formulation of the examples, 0.9 molar parts (=molar fraction in the left part of column 19) of the first amphipat are mixed with the “Average mix” of the second and third amphipat specified in columns 16 and 17 as well as 25 and 26, MW-wise. To prepare the overall mixture, one should use 89.59 g L⁻¹ of the first amphipat (left part of column 21) with 4EG (column 12) and MW=362 (column 23) plus 10.4 g L⁻¹ (right part of column 21) of the “Average mix”. The latter should consist of a 0.85 molar fraction (column 16, not shown) of the second amphipat (which, in this Example, is identical to the first amphipat (compare column 14 with column 12; column 9 with 11; and column 23 with 25) and a 0.15 molar fraction (column 17, not shown) of the third, charged, amphipat, which in this embodiment is also a single-chain (column 8) lauryl (column 6) ether (column 11) with 6EG (column 15) and a sulphate group (cf. column 1) attached thereto. To calculate the required absolute concentration of the third amphipat, one needs to multiply its molar fraction, 0.15 (column 16, not shown), with its relative molar mass (i.e. the ratio of values given in columns 26 and 24). Correspondingly, to achieve the required (extra) concentration of the second amphipat, one must multiply its molar fraction in the “Average mix”, 0.85 (column 16, not shown), with the second amphipat relative molar mass (the ratio of values in columns 25 and 24, not shown). This yields the effective molar ratio of the first and the putative “Average mix” amphipat of 9/1 (column 20).

Additional embodiments of the invention are specified and prepared similarly. For avoidance of doubt, and as another detailed example, Example 112, is made of: i) a first amphipat (a mono-oleoyl-EG2-ether at concentration of 73.44 g L⁻¹, wide column 21); ii) as part of the “Average mix”, a second amphipat (a mono-oleoyl-EG10-ether at concentration of 24.27 g L⁻¹=0.83×26.56×710/648, the dividend corresponding to the second amphipat molar mass and the divisor to the average molar mass of the putative “Average mix”); iii) also as part of the “Average mix”, a third amphipat (a mono-oleyl-phosphate is used at an absolute concentration of 1.74 g L⁻¹=0.17×26.56×335/648). (Column 13 gives the calculated resulting effective headgroup length in the mix, but only provides a general orientation as the employed fatty-phosphate is not ethoxylated.) Provision of other embodiments according to the invention follows identical rules.

If the chosen charged (third) amphipat compromises bilayer stability, such charged amphipat of the single-chain type (such as oleylphosphate or hexadecyl-sulphate of Examples 110, 112-115) can be replaced by the corresponding, or some other, charged double-chain amphipat (such as dicetylphosphate of Examples 111 and 117). If an amphipat cannot be used at the desired pH (e.g. owing to insufficient ionisation at the pH), a different ionisable group with a higher or lower pKa, as required, is chosen. For example, a glutamate-, sarcosinate-, carboxylate, etc. is used instead of phosphate or sulphate headgroup of the non-limiting Examples provided in Table 2.

Considering the common use of phosphatides in vesicular formulations, a charged phospholipid, i.e. phosphatidylglycerol, is included in Examples 120, 123 and 124. This phosphatide is also attractive due to its quasi-ideal miscibility with phosphatidylcholine. When analyzing a suspension prepared from hydrolysable (e.g., ester-based) lipids, one should consider charged lipid degradation products, amongst which fatty acids are the most prominent. As an example for a potentially resulting suspension, an equimolar blend of lysophosphatidylcholine and oleic acid is provided in Table 2.

### 6.3 Oil containing Formulations

Tables 1 and 2 also report several embodiments containing one relatively apolar (oily) substance, a fatty alcohol or a fatty acid in acidic preparations. Other Examples containing oils can be designed according to the guidance provided herein.

### 6.4 Preparations with Various Additives

The Examples specified in Tables 1 and 2 are minimalistic in that they contain a few select amphipats and a buffer, with constant total amphipats and buffer concentration. Suspensions of otherwise similar compositions made with various buffers or somewhat higher or lower total amphipat concentration, or select co-solvents, can produce similar results, provided that the average area per chain and/or polarity units count and/or HLB value are in the range specified herein. All the preparations specified in Table 1 and all the preparations with pH=7.5 specified in Table 2 can be made in a 15 mM or 150 mM phosphate buffer, for example, presuming charged amphipat ionisation. Inclusion of at least up to around 1 wt.-% ethanol or up to around 0.5 wt.-% benzyl alcohol does not detrimentally affect the proposed formulations stability, although it does increase aggregate adaptability and somewhat lowers formulation stability. Higher alcohol concentration or addition of further co-solvents can require lowering some other formulation component concentrations. Ac-increasing compounds are especially useful in the context. In turn, inclusion of rather bulky additives with a high octanol-water partition coefficient into a formulation is apt to diminish the effective area per amphipat chain and thus disadvantageously stiffen the formulation bilayers. Propyl- or butyl-parabene, e.g., have this tendency if incorporated into aggregates of the invention in an appreciable quantity.

Further additives introduction into a preparation of the invention can cause similar phenomena. Table 3 provides an overview of the most suitable concentrations for a series of popular and broadly useful additives according to the invention, and refers to several Examples included in Table 1. Collectively, the data provided in Tables 1 to 3, together with the rules and guidance described herein, advantageously permit rapid production of pharmaceutically acceptable formulations capable of imparting therapeutic benefit to a subject, in particular in the treatment of pain and inflammation.

TABLE 1
Table 1A. Non-limiting exemplary compositions of nonionic aggregates of the invention, as described in the text (PART A),
illustrating composition effect on the effective area per hydrophobic chain, Ac, the HLB number, and the calculated effective number
of polar segments per headgroup, “nEGe”.

| | | | | | | | | | | | | | | | | | | | |
|---|---|---|---|---|---|---|---|---|---|---|---|---|---|---|---|---|---|---|---|
| Column | 1 | 2 | 3 | 4 | 5 | 6 | 7 | 8 | 9 | 10 | 11 | 12 | 13 | 14 | 15 | 18 | 23 | 24 | 25 |

| | | | | | |
|---|---|---|---|---|---|
| Amphipat characteristics | Amphipats | Aggregate compos. | Ac | nP | HLB |

| | | | | | | | | |
|---|---|---|---|---|---|---|---|---|
| # | 1 | 2 | 3 | 4 | 1 | 2 | 3 | 4 |

| | | | | | | | | | | |
|---|---|---|---|---|---|---|---|---|---|---|
| Chains length, number, type | Head | Head | Hea [symbol] | Hea [symbol] | Head | Head | Head | Head | #1/#2 | pH |

| | | | | | | |
|---|---|---|---|---|---|---|
| L₁ | DB₁ | n₁ | n₂ | n₃ | DB_{2.3}^{a} | w + w |

| | | | | | | | | |
|---|---|---|---|---|---|---|---|---|
| Emb. | Polyoxyethyleneglycol (pEG) | Bond type | EG/n₁ | EG/n₂ | [symbol] G/n₃ | [symbol] G/n₄ | g L⁻¹ | g L⁻¹ |

| | | |
|---|---|---|
| Nr. | direct aryl chain attachment*, linear headgroup | “Average” mix |

| 1 | C | 8phenol | 1 | 1 | | | ETc | ETc | 3.0 | 7.5 | Triton X-35 | Triton X-114 | 60.0 | 40.00 | 7.4 | 0.44 | 4.3 | 9.2 |
|---|---|---|---|---|---|---|---|---|---|---|---|---|---|---|---|---|---|---|
| 2 | C | 9phenol | 1 | 1 | | | ETc | ETc | 4.0 | 7.0 | Tergitol NP-4 | Tergitol NP-7 | 60.0 | 40.00 | | 0.46 | 4.9 | 9.7 |
| | direct aliphatic chains attachment*, linear headgroup | | | | | | | | | | | | | | | | | |
| 3 | C | 12 | 1 | 1 | | | ET | ET | 0.0 | 9.0 | Dodecanol | Emalex 709 | 20.0 | 80.00 | 7.4 | 0.40 | 4.6 | 6.7 |
| 4 | C | 12 | 1 | 0 | | | ET | | 4.0 | [symbol] | Brij 30 | | 100.0 | 0.00 | 7.4 | 0.42 | 4.0 | 9.6 |
| 5 | C | 12 | 1 | 1 | | | ET | ET | 3.0 | 5.0 | Emalex 703 | Emalex 705 | 60.0 | 40.00 | 7.4 | 0.41 | 3.7 | 8.2 |
| 6 | C | 16/18 | 1 | 1 | | | ET | ET | 5.0 | 10.0 | Emalex 1605 | Emalex 1810 | 70.0 | 30.00 | 7.4 | 0.47 | 6.1 | 10.6 |
| 7 | C | 18 i | 1 | 1 | | | ET | ET | 5.0 | 10.0 | Emalex 1805 | Emalex 1810 | 80.0 | 20.00 | 7.4 | 0.46 | 5.7 | 9.5 |
| 8 | C | 18:1 | 1 | 1 | | | ET | ET | 0.0 | 10.0 | Cteyl alcohol | Brij 97 | 24.0 | 76.00 | 7.4 | 0.39 | 5.5 | 10.4 |
| 9 | C | 18:1 | 1 | 1 | | | ET | ET | −0.5 | 10.0 | Oleic acid | Brij 97 | 24.0 | 76.00 | 6.5 | 0.38 | 5.0 | 8.9 |
| 10 | C | 18:1 | 1 | 1 | | :1 | ET | ET | 2.0 | 10.0 | Brij 93 | Brij 97 | 60.0 | 40.00 | 7.4 | 0.40 | 4.0 | 6.8 |
| 11 | C | 18:1 | 1 | 1 | | :1 | ET | ET | 2.0 | 10.0 | Brij 93 | Brij 97 | 40.0 | 60.00 | 7.4 | 0.44 | 5.4 | 8.1 |
| 12 | C | 18:1 | 1 | 1 | | :1 | ET | ET | 2.0 | 10.0 | Brij 93 | Brij 97 | 29.0 | 71.00 | 7.4 | 0.47 | 6.4 | 9.0 |
| 13 | C | 18:1 | 1 | 1 | | :1 | ET | ET | 2.0 | 10.0 | Brij 93 | Brij 97 | 25.0 | 75.00 | 7.4 | 0.48 | 6.8 | 9.4 |
| 14 | C | 18:1 | 1 | 1 | C12 | :1 | ET | ET | 2.0 | 5.0 | Brij 93 | Emalex 705 | 15.0 | 85.00 | 7.4 | 0.42 | 4.2 | 7.9 |
| 15 | C | 18:1 | 1 | 1 | | | ES | ES | 4.0 | 13.0 | C18:1EO4 | C18:1EO13 | 60.0 | 40.00 | 7.4 | 0.44 | 5.9 | 7.7 |
| 16 | C | 18:1 | 1 | 1 | | | ES | ES | 4.0 | 13.0 | C18:1EO4 | C18:1EO13 | 50.0 | 50.00 | 7.4 | 0.46 | 6.6 | 8.0 |
| 17 | C | 18:1 | 1 | 1 | | | ES | ES | 4.0 | 13.0 | C18:1EO4 | C18:1EO13 | 40.0 | 60.00 | 7.4 | 0.47 | 7.5 | 8.4 |
| 18 | C | 18:1 | 1 | 0 | | | ET | | 5.5 | [symbol] | Emuil [symbol] LP | | 100.0 | 0.00 | 7.4 | 0.46 | 5.5 | 9.3 |
| 19 | C | 18:1 | 1 | 1 | | :1 | ET | ET | 5.5 | 10.0 | Emuil [symbol] LP | Brij 97 | 70.0 | 30.00 | 7.4 | 0.48 | 6.6 | 10.0 |
| 20 | C | 18:1 | 1 | 1 | | :1 | ES | ES | 3.0 | 6.0 | Emalex 218 | Ematex OE6 | 10.0 | 90.00 | 7.4 | 0.43 | 5.1 | 9.2 |
| 21 | C | 18:1 | 1 | 1 | | :1 | ES | ES | 3.0 | 8.0 | Emalex 218 | Cithrol 4MO | 30.0 | 70.00 | 7.4 | 0.44 | 5.5 | 9.1 |
| 22 | C | 18:OH | 1 | 1 | | CH | ET/S | ET/S | 5.0 | 10.0 | Emalex HC-5 | Emalex HC-1 [symbol] | 60.0 | 40.00 | 7.4 | 0.45 | 6.6 | 10.1 |
| 23 | C | 18:1 | 1 | 1 | | | AD | ET | 7.0 | 5.0 | Ethomid O/17 | Emalex 1805 | 10.0 | 90.00 | 7.4 | 0.43 | 5.2 | 9.1 |

| | indirect aliphatic chain(s) attachment | | | | | | | | | | | | | | | | | |
|---|---|---|---|---|---|---|---|---|---|---|---|---|---|---|---|---|---|---|
| | sorbitan, branched headgroup; stochastic aliphatic (“anchor”) chains distribution | | | | | | | | | | | | | | | | | |
| 24 | C | 12 | 1 | 1 | | | SES | SES | 0.0 | 20.0 | Span 20 | Tween 20 | 50.0 | 50.00 | 7.4 | 0.34 | 2.6 | 10.4 |
| 25 | C | 12 | 1 | 0 | | | SES | | 4.0 | [symbol] | Tween 21 | | 100.0 | 0.00 | 7.4 | 0.41 | 2.4 | 11.0 |
| 26 | C | 12 | 1 | 1 | 18 | :1 | SES | SES | 4.0 | −0.5 | Tween 21 | Oleic acid | 87.0 | 13.00 | 7.8 | 0.40 | 1.7 | 8.6 |

Table 1B. Non-limiting exemplary compositions of the invention, as described in the text (PART B)

| | | | | | | | | | | | | | | | | | | | |
|---|---|---|---|---|---|---|---|---|---|---|---|---|---|---|---|---|---|---|---|
| Column | 1 | 2 | 3 | 4 | 5 | 6 | 7 | 8 | 9 | 10 | 11 | 12 | 13 | 14 | 15 | 18 | 23 | 24 | 25 |

| | | | | | |
|---|---|---|---|---|---|
| Amphipat characteristics | Amphipats | Aggregate compos. | Ac | nP | HLB |

| | | | | | | | | |
|---|---|---|---|---|---|---|---|---|
| # | 1 | 2 | 3 | 4 | 1 | 2 | 3 | 4 |

| | | | | | | | | | | |
|---|---|---|---|---|---|---|---|---|---|---|
| Chains length, number, type | Head | Head | Hea [symbol] | Hea [symbol] | Head | Head | Head | Head | #1/#2 | pH |

| | | | | | | | |
|---|---|---|---|---|---|---|---|
| Emb. | L₁ | DB₁ | n₁ | n₂ | n₃ | DB_{2.3}^{a} | w + w |

| Nr. | Polyoxyethyleneglycol (pEG) | | | | | Bond type | | | EG/n₁ | EG/n₂ | [symbol] G/n₃ | [symbol] G/n₄ | | | | | | | g L⁻¹ | g L⁻¹ | | | | |
|---|---|---|---|---|---|---|---|---|---|---|---|---|---|---|---|---|---|---|---|---|---|---|---|---|
| | indirect aliphatic chain(s) attachment | | | | | | | | | | | | | | | | | | | | | | | |
| | sorbitan, branched headgroup; stochastic aliphatic (“anchor”) chains distribution | | | | | | | | | | | | | | | | | | | | | | | |
| 27 | C | 18:1 | 1 | | | SES | | | 0.0 | 20.0 | | | Span 80 | | | | | | 100.0 | 0.00 | 7.4 | 0.18 | 0.0 | 4.3 |
| 28 | C | 18:1 | 1 | 1 | :1 | SES | SES | | 0.0 | 20.0 | | | Span 80 | Tween 80 | | | | | 90.0 | 10.00 | 7.4 | 0.19 | 0.4 | 4.7 |
| 29 | C | 18:1 | 1 | 1 | :1 | SES | SES | | 0.0 | 20.0 | | | Span 80 | Tween 80 | | | | | 75.0 | 25.00 | 7.4 | 0.22 | 1.2 | 5.4 |
| 30 | C | 18:1 | 1 | 1 | :1 | SES | SES | | 0.0 | 20.0 | | | Span 80 | Tween 80 | | | | | 60.0 | 40.00 | 7.4 | 0.25 | 2.1 | 6.2 |
| 31 | C | 18:1 | 1 | 1 | :1 | SES | SES | | 0.0 | 20.0 | | | Span 80 | Tween 80 | | | | | 50.0 | 50.00 | 7.4 | 0.28 | 2.9 | 6.9 |
| 32 | C | 18:1 | 1 | 1 | :1 | SES | SES | | 0.0 | 20.0 | | | Span 80 | Tween 80 | | | | | 35.0 | 65.00 | 7.4 | 0.34 | 4.5 | 8.4 |
| 33 | C | 18:1 | 1 | 1 | :1 | SES | SES | | 0.0 | 20.0 | | | Span 80 | Tween 80 | | | | | 25.0 | 75.00 | 7.4 | 0.38 | 9.9 | 9.6 |
| 34 | C | 18:1 | 1 | 1 | :1 | SES | SES | | 0.0 | 20.0 | | | Span 80 | Tween 80 | | | | | 17.5 | 82.50 | 7.4 | 0.43 | 7.2 | 10.8 |
| 35 | C | 18:1 | 1 | | | SES | | | 5.0 | | | | Tween 81 | | | | | | 100.0 | 0.00 | 7.4 | 0.39 | 2.9 | 10.0 |
| 36 | C | 18:1 | 1 | 1 | :1 | SES | SES | | 5.0 | 20.0 | | | Tween 81 | Tween 80 | | | | | 80.0 | 20.00 | 7.4 | 0.41 | 3.9 | 10.6 |
| 37 | C | 18:1 | 1 | 1 | :1 | SES | SES | | 5.0 | 20.0 | | | Tween 81 | Tween 80 | | | | | 70.0 | 30.00 | 7.4 | 0.42 | 4.5 | 10.9 |
| 38 | C | 18:1 | 1 | 1 | :1 | SES | SES | ET | 5.0 | 8.0 | 20 | 20 | Tween 81 | Average | Tween 80 | Brij 98 | 0.50 | 0.50 | 9.57 | 90.4 | 7.4 | 0.46 | 7.2 | 14.3 |
| 39 | C | 18:1 | 1 | 1 | :1 | SES | SES | | 5.0 | 20.0 | | | Tween 81 | Tween 80 | | | | | 50.0 | 50.00 | 7.4 | 0.45 | 5.9 | 11.7 |
| 40 | C | 18:1 | 1 | 1 | :1 | SES | SES | | 5.0 | 20.0 | | | Tween 81 | Tween 80 | | | | | 30.0 | 70.00 | 7.4 | 0.50 | 7.7 | 12.7 |
| 41 | C | 18:1 | 3.0 | 1 | :1 | SES | SES | | 20.0 | 0.0 | | | Tween 85 | Span 80 | | | | | 82.0 | 18.00 | 7.4 | 0.30 | 3.4 | 7.7 |
| 42 | C | 18:1 | 3.0 | 0 | | SES | | | 20.0 | 0.0 | | | Tween 85 | | | | | | 100.0 | 0.00 | 7.4 | 0.42 | 3.9 | 11.0 |
| 43 | C | 18:1 | 3.0 | 1 | :1 | SES | SES | | 20.0 | 20.0 | | | Tween 85 | Tween 80 | | | | | 70.0 | 30.00 | 7.4 | 0.48 | 6.9 | 12.5 |
| 44 | C | 18:1 | 3.0 | 1 | :1 | SES | SES | | 20.0 | 20.0 | | | Tween 85 | Tween 80 | | | | | 50.0 | 50.00 | 7.4 | 0.52 | 8.5 | 13.3 |

| Polyglyceryl (pG) | | | | | nG nG/nEG | | | | | | | | | | | | | |
|---|---|---|---|---|---|---|---|---|---|---|---|---|---|---|---|---|---|---|
| | multiple aliphatic “anchor” chain attachment options/(stochastic) distribution | | | | | | | | | | | | | | | | | |
| 45 | C | 12 | 1 | | | | ES-G | | 5.0 | — | Dermofeel G 5 L | | 100.0 | 0.01 | 7.4 | 0.51 | 8.2 | 13.0 |
| 46 | C | 12 | 1 | 1 | C18 | :1 | ES-G | [symbol] | 5.0 | 0.0 | Dermofeel G 5 L | Octedecomol | 90.0 | 10.00 | 7.4 | 0.46 | 6.5 | 12.1 |
| 47 | C | 18:1 | 1 | | | | ES-G | | 5.0 | — | Dermofeel G 5O | | 100.0 | 0.01 | 7.4 | 0.52 | 8.2 | 11.5 |
| 48 | C | 18:1 | 1.6 | | | | ES-G | | 5.0 | [symbol] | Dermofeel G 5 DO | | 100.0 | 0.01 | 7.4 | 0.45 | 5.1 | 8.0 |
| 49 | C | 18:1 | 1 | 1.6 | C18 | :1 | ES-G | ES-G | 5.0 | 5.0 | Dermofeel G 5O | Dermofeel G 5 DO | 75.0 | 25.00 | 7.4 | 0.51 | 7.5 | 10.8 |
| 50 | C | 18:1 | 1 | | | | ES-G | | 2.0 | [symbol] | Emulsogen OG; Er | | 100.0 | 0.00 | 7.4 | 0.25 | 0.3 | 8.0 |
| 51 | C | 18:1 | 1 | 1 | | :1 | ES-G | SES | 2.0 | 20.0 | Emulsogen OG; Er | Tween 80 | 90.0 | 10.00 | 7.4 | 0.26 | 0.7 | 8.3 |
| 52 | C | 18:1 | 1 | 1 | | :1 | ES-G | SES | 2.0 | 20.0 | Emulsogen OG; Er | Tween 80 | 80.0 | 20.00 | 7.4 | 0.28 | 1.2 | 8.6 |
| 53 | C | 18:1 | 1 | 1 | | :1 | ES-G | SES | 2.0 | 20.0 | Emulsogen OG; Er | Tween 80 | 70.0 | 30.00 | 7.4 | 0.29 | 1.8 | 8.9 |
| 54 | C | 18:1 | 1 | 1 | | :1 | ES-G | SES | 2.0 | 20.0 | Emulsogen OG; Er | Tween 80 | 50.0 | 50.00 | 7.4 | 0.34 | 3.2 | 9.8 |
| 55 | C | 18:1 | 1 | 1 | | :1 | ES-G | SES | 2.0 | 20.0 | Emulsogen OG; Er | Tween 80 | 25.0 | 75.00 | 7.4 | 0.42 | 6.1 | 11.5 |
| 56 | C | 18:1 | 1 | 1 | | :1 | ES-G | SES | 2.0 | 20.0 | Emulsogen OG; Er | Tween 80 | 15.0 | 85.00 | 7.4 | 0.47 | 7.9 | 12.6 |

Table 1C. Non-limiting exemplary compositions of the invention, as described in the text (PART C)

| | | | | | | | | | | | | | | | | | | | |
|---|---|---|---|---|---|---|---|---|---|---|---|---|---|---|---|---|---|---|---|
| Column | 1 | 2 | 3 | 4 | 5 | 6 | 7 | 8 | 9 | 10 | 11 | 12 | 13 | 14 | 15 | 18 | 23 | 24 | 25 |

| | | | | | |
|---|---|---|---|---|---|
| Amphipat characteristics | Amphipats | Aggregate compos. | Ac | nP | HLB |

| | | | | | | | | |
|---|---|---|---|---|---|---|---|---|
| # | 1 | 2 | 3 | 4 | 1 | 2 | 3 | 4 |

| | | | | | | | | | | | |
|---|---|---|---|---|---|---|---|---|---|---|---|
| Emb. | Chains length, number, type | Head | Head | Hea [symbol] | Hea [symbol] | Head | Head | Head | Head | #1/#2 | pH |

| | | | | | | | |
|---|---|---|---|---|---|---|---|
| Nr. | L₁ | DB₁ | n₁ | n₂ | n₃ | DB_{2.3}^{a} | w + w |

| Polyglyceryl (pG) | | | | | nG nG/nEG | | | | | | | | | | | | | | | | | | | | |
|---|---|---|---|---|---|---|---|---|---|---|---|---|---|---|---|---|---|---|---|---|---|---|---|---|---|
| | multiple aliphatic “anchor” chain attachment options/(stochastic) distribution | | | | | | | | | | | | | | | | | | | | | | | | |
| 57 | C | 18:1 | 1 | 1 | | :1 | ES-G | ES-G | | 2.0 | 5.0 | | | Emulsogen OG: E [symbol] | Dermofeel G 5O | | | | | 15.0 | 85.00 | 7.4 | 0.46 | 6.5 | 13.5 |
| 58 | C | 18:1 | 1.5 | | | :1 | ES-G | | | 10.0 | — | | | Caprol PGE 860 | | | | | | 100.0 | 0.00 | 7.4 | 0.31 | 1.1 | 11.0 |
| 59 | C | 18:1 | 1.5 | 1 | | :1 | ES-G | SES | | 10.0 | 20.0 | | | Caprol PGE 860 | Tween 80 | | | | | 50.0 | 50.00 | 7.4 | 0.45 | 6.4 | 13.0 |
| | (Poly)Sugar derivatives (here, (poly)hexose) | | | | | | | | | nₕₑₓ | nₕₑₓ | | | | | | | | | | | | | | |
| | multiple “anchor” chain attachment options/(stochastic) distribution | | | | | | | | | | | | | | | | | | | | | | | | |
| 60 | C | 12 | 1 | 1 | | | SES | ES | | 0 | 1.0 | | | Span 20 | C12Glu | | | | | 10.0 | 90.00 | 7.4 | 0.43 | 3.6 | 14.1 |
| 61 | C | 12 | 5.4 | 1.7 | | | ES | ES | | 0.4 | 1.2 | | | Surfhope C-1201/ | Surfhope C-1205/L-595 | | | | | 50.0 | 50.00 | 7.4 | 0.38 | 2.0 | 3.7 |
| 62 | C | 12 | 1.7 | | | | ES | | | 1.2 | — | | | Surfhope C-1205/L-595 | | | | | | 100.0 | 0.00 | 7.4 | 0.41 | 2.8 | 5.0 |
| 63 | C | 12 | 1.7 | 1.2 | | | ES | ES | | 1.2 | 1.7 | | | Surfhope C-1205/ | Surfhope C-1216/Ryoto L-1695 | | | | | 50.0 | 50.00 | 7.4 | 0.45 | 4.5 | 11.0 |
| 64 | C | 18:1 | 5 | 1 | | :1 | ES | ES | | 0.4 | 1.7 | | | Surfhope C-1701 | Surfhope C-1715 | | | | | 75.0 | 25.00 | 7.4 | 0.36 | 3.2 | 7.2 |
| 65 | C | 18:1 | 5 | 1 | | :1 | ES | ES | | 0.4 | 1.7 | | | Surfhope C-1701 | Surfhope C-1715 | | | | | 50.0 | 50.00 | 7.4 | 0.42 | 4.9 | 10.9 |
| 66 | C | 18:1 | 5 | 1 | | :1 | ES | ES | | 0.4 | 1.7 | | | Surfhope C-1701 | Surfhope C-1715 | | | | | 25.0 | 75.00 | 7.4 | 0.46 | 6.0 | 13.3 |
| | Phosphatides | | | | | | | | | | | | | | | | | | | | | | | | |
| | P-head^{c} EO | | | | | | | | | | | | | | | | | | | | | | | | |
| 72 | C | 18:2 | 2 | 1 | | :1 | ES | ET | | 4.0 | 20.0 | | | SPC | Brij 98 | | | | | 87.57 | 12.4 | 7.4 | 0.38 | 3.6 | 8.7 |
| 73 | C | 18:2 | 2 | 1 | | :1 | ES | ET | | 4.0 | 20.0 | | | SPC | Brij 98 | | | | | 84.93 | 15.1 | 7.4 | 0.39 | 4.0 | 8.8 |
| 74 | C | 18:2 | 2 | 1 | | :1 | ES | ET | | 4.0 | 20.0 | | | SPC | Brij 98 | | | | | 80.87 | 19.1 | 7.4 | 0.40 | 4.6 | 9.0 |
| 75 | C | 18:2 | 2 | 1 | | :1 | ES | ET | | 4.0 | 20.0 | | | SPC | Brij 98 | | | | | 77.88 | 22.1 | 7.4 | 0.41 | 5.0 | 9.2 |
| 76 | C | 18:2 | 2 | 1 | | :1 | ES | ET | | 4.0 | 20.0 | | | SPC | Brij 98 | | | | | 73.80 | 26.2 | 7.4 | 0.42 | 5.6 | 9.5 |
| 77 | C | 18:2 | 2 | 1 | | :1 | ES | ET | | 4.0 | 20.0 | | | SPC | Brij 98 | | | | | 67.88 | 32.1 | 7.4 | 0.44 | 6.5 | 9.8 |
| 78 | C | 18:2 | 2 | 1 | | :1 | ES | ET | | 4.0 | 20.0 | | | SPC | Brij 98 | | | | | 58.48 | 41.5 | 7.4 | 0.47 | 8.0 | 10.4 |
| 79 | C | 18:2 | 2 | 1 | | :1 | ES | ET | | 4.0 | 20.0 | | | SPC | Brij 98 | | | | | 41.33 | 58.7 | 7.4 | 0.53 | 11.0 | 11.7 |
| 80 | C | 18:2 | 2 | 1 | | :1 | ES | ET | | 4.0 | 20.0 | | | SPC | Brij 98 | | | | | 26.05 | 74.0 | 7.4 | 0.59 | 14.0 | 12.9 |
| 81 | C | 18:2 | 2 | 1 | 1 | :1 | ES | ES-G | ET | 4.0 | 8.0 | 5.5 | 10 | SPC | Average | Emuilsoger | Brij 97 | 0.44 | 0.56 | 51.02 | 49.0 | 7.4 | 0.44 | 5.3 | 9.7 |
| 82 | C | 18:2 | 2 | 1 | | :1 | ES | ET | | 4.0 | 6.5 | | | SPC | Emalex CE-6 | | | | | 26.70 | 73.3 | 7.4 | 0.46 | 5.6 | 9.3 |

| | | | | | | | | | | | | | | | | | |
|---|---|---|---|---|---|---|---|---|---|---|---|---|---|---|---|---|---|
| 83 | C | 18:2 | 2 | 1 | :1 | ES | ES | 4.0 | 7.5 | SPC | Simulsol-2599; Macrogol 10-oleate | 50.56 | 49.4 | 7.4 | 0.43 | 4.6 | 10.1 |
| 84 | C | 18:2 | 2 | 1 | :1 | ES | SES | 4.0 | 20.0 | SPC | Tween 80 | 88.78 | 11.2 | 7.4 | 0.37 | 2.7 | 8.5 |
| 85 | C | 18:2 | 2 | 1 | :1 | ES | SES | 4.0 | 20.0 | SPC | Tween 80 | 81.22 | 18.8 | 7.4 | 0.38 | 3.2 | 8.9 |
| 86 | C | 18:2 | 2 | 1 | :1 | ES | SES | 4.0 | 20.0 | SPC | Tween 80 | 71.19 | 28.8 | 7.4 | 0.40 | 4.0 | 9.4 |
| 87 | C | 18:2 | 2 | 1 | :1 | ES | SES | 4.0 | 20.0 | SPC | Tween 80 | 64.96 | 35.0 | 7.4 | 0.41 | 6.5 | 9.8 |
| 88 | C | 18:2 | 2 | 1 | :1 | ES | SES | 4.0 | 20.0 | SPC | Tween 80 | 60.70 | 39.3 | 7.4 | 0.42 | 4.8 | 10.0 |

Table 1D. Non-limiting exemplary compositions of the invention, as described in the text (PART D)

| | | | | | | | | | | | | | | | | | | | |
|---|---|---|---|---|---|---|---|---|---|---|---|---|---|---|---|---|---|---|---|
| Column | 1 | 2 | 3 | 4 | 5 | 6 | 7 | 8 | 9 | 10 | 11 | 12 | 13 | 14 | 15 | 18 | 23 | 24 | 25 |

| | | | | | |
|---|---|---|---|---|---|
| Amphipat characteristics | Amphipats | Aggregate compos. | Ac | nP | HLB |

| | | | | | | | | |
|---|---|---|---|---|---|---|---|---|
| # | 1 | 2 | 3 | 4 | 1 | 2 | 3 | 4 |

| | | | | | | | | | | | |
|---|---|---|---|---|---|---|---|---|---|---|---|
| Emb. | Chains length, number, type | Head | Head | Hea [symbol] | Hea [symbol] | Head | Head | Head | Head | #1/#2 | pH |

| | | | | | | | |
|---|---|---|---|---|---|---|---|
| Nr. | L₁ | DB₁ | n₁ | n₂ | n₃ | DB_{2.3}^{a} | w + w |

| | Phosphatides | | | | | | | | | | | | | | | | | | | | | | | | |
|---|---|---|---|---|---|---|---|---|---|---|---|---|---|---|---|---|---|---|---|---|---|---|---|---|---|
| | P-head^{c} EO | | | | | | | | | | | | | | | | | | | | | | | | |
| 89 | C | 18:2 | 2 | 1 | | :1 | ES | SES | | 4.0 | 20.0 | | | SPC | Tween 80 | | | | | 55.27 | 44.7 | 7.4 | 0.43 | 8.0 | 10.3 |
| 90 | C | 18:2 | 2 | 1 | | :1 | ES | SES | | 4.0 | 20.0 | | | SPC | Tween 80 | | | | | 38.19 | 61.8 | 7.4 | 0.47 | 6.9 | 11.5 |
| 91 | C | 18:2 | 2 | 1 | | :1 | ES | SES | | 4.0 | 20.0 | | | SPC | Tween 80 | | | | | 23.60 | 76.4 | 7.4 | 0.51 | 14.0 | 12.7 |
| 92 | C | 18:2 | 2 | 1 | | :1 | ES | SES | SES | 4.0 | 10.0 | 20.0 | 0 | SPC | Average | Tween 80 | Span 80 | 0.50 | 0.50 | 42.66 | 57.3 | 7.4 | 0.42 | 4.2 | 8.9 |
| 93 | C | 18:2 | 2 | 1 | 1 | :1 | ES | SES | SES | 4.0 | 12.0 | 20.0 | 0 | SPC | Average | Tween 80 | Span 80 | 0.60 | 0.40 | 35.44 | 64.6 | 7.4 | 0.44 | 5.1 | 9.6 |
| 94 | C | 18:2 | 2 | 1 | 1 | :1 | ES | SES | SES | 4.0 | 14.0 | 20.0 | 0 | SPC | Average | Tween 80 | Span 80 | 0.70 | 0.30 | 27.89 | 72.1 | 7.4 | 0.47 | 6.2 | 10.5 |
| 95 | C | 18:2 | 2 | 1 | 1 | :1 | ES | SES | SES | 4.0 | 8.0 | 20.0 | 0 | SPC | Average | Tween 80 | Span 80 | 0.40 | 0.60 | 29.27 | 70.7 | 7.4 | 0.41 | 3.9 | 8.4 |
| 96 | C | 18:2 | 2 | 1 | 1 | :1 | ES | SES | SES | 4.0 | 9.0 | 20.0 | 0 | SPC | Average | Tween 80 | Span 80 | 0.45 | 0.55 | 28.15 | 71.9 | 7.4 | 0.43 | 4.4 | 8.8 |
| 97 | C | 18:2 | 2 | 1 | 1 | :1 | ES | ET | ET | 4.0 | 8.0 | 2.0 | 10 | SPC | Average | Brij 93 | Brij 97 | 0.25 | 0.75 | 51.02 | 49.0 | 7.4 | 0.44 | 5.3 | 9.4 |
| 98 | C | 18:2 | 2 | 1 | 1 | :1 | ES | ET | ET | 4.0 | 6.0 | 2.0 | 10 | SPC | Average | Brij 93 | Brij 97 | 0.50 | 0.50 | 31.27 | 68.7 | 7.4 | 0.45 | 5.1 | 8.5 |
| 99 | C | 18:1 | 2 | 1 | | :1 | ES | SES | | 2.8 | 20.0 | | | DOPE2M | Tween 80 | | | | | 35.31 | 64.7 | 7.4 | 0.46 | 6.9 | 10.7 |
| 100 | C | 18:1 | 2 | 1 | | :1 | ES | SES | SES | 2.8 | 11.0 | 20.0 | 5.0 | DOPE2M | Average | Tween 80 | Tween 80 | 0.40 | 0.60 | 25.69 | 74.3 | 7.4 | 0.43 | 4.8 | 12.0 |
| 101 | C | 18:1 | 2 | 1 | 1 | :1 | ES | SES | SES | 2.8 | 11.0 | 20.0 | 0.0 | DOPE2M | Average | Tween 80 | Span 80 | 0.55 | 0.45 | 25.69 | 74.3 | 7.4 | 0.43 | 4.8 | 12.0 |
| 102 | C | 18:1 | 2 | 1 | 1 | :1 | ES | ET | ET | 2.8 | 8.0 | 2.0 | 20.0 | DOPE2M | Average | Brij 93 | Brij 98 | 0.67 | 0.33 | 52.61 | 47.4 | 7.4 | 0.45 | 5.8 | 7.6 |
| 103 | C | 18:1 | 2 | 1 | 1 | :1 | ES | ET | ET | 2.8 | 7.0 | 3.0 | 20.0 | DOPE2M | Average | Brij 93 | Brij 98 | 0.72 | 0.28 | 52.61 | 47.4 | 7.4 | 0.44 | 5.1 | 7.2 |
| 104 | C | 18:2 | 1 | 1 | 1 | :1 | ES | ES | | 4.5 | 2.0 | −0.5 | 0.0 | lyso-SPC | Oleic acid | | | | | 65.19 | 34.8 | 6.5 | 0.39 | 3.0 | 6.5 |
| 105 | C | 18:2 | 1 | 1 | 1 | :1 | ES | (ET) | ET | 4.5 | 6.0 | 0.0 | 20.0 | lyso-SPC | Average | 0.5 Oleic [symbol] | Brij 98 | 0.70 | 0.30 | 62.61 | 37.4 | 6.5 | 0.46 | 5.3 | 11.5 |
| 106 | C | 18:2 | 1 | 1 | 1 | :1 | ES | ES | ET | 4.5 | 6.0 | −0.5 | 20.0 | lyso-SPC | Average | 0.5 Oleic [symbol] | Brij 98 | 0.68 | 0.32 | 52.91 | 47.1 | 6.5 | 0.46 | 5.2 | 10.0 |
| 107 | C | 18:2 | 1 | 1 | 1 | :1 | ES | ES | ET | 4.5 | 4.9 | −0.6 | 0.0 | lyso-SPC | Average | 0.5 Oleic [symbol] | Brij 98 | 0.02 | 0.98 | 42.82 | 57.2 | 6.5 | 0.44 | 4.8 | 14.5 |

*ETc = alkylphenolether;
ET = ether;
ET* = ether with relatively narrow head-length distribution;
ES = ester;
SES = sorbitan-ester;
AD = amide;
ES-G = ester of a polyglyceryl
**C..i = iso-branching: —OH hydrogenated ricin chain; (ET) fatty alcohol +C18:1polysorbate
^{a}If not otherwise specified, all amphipats have similar chains, on the average.
^{b}For a 3 component mixture, Xn2 gives molar sum of the 2nd and 3rd component.
^{c}MW for di-chain phosphatidylcholines (PC/SPC) is given for the prevailing dihydrate [symbol]
indicates data missing or illegible when filed

TABLE 2
Non-exclusive listing of charged aggregate compositions of the invention, as described in the text.

| Column Nr. | 1 | 2 | 3 | 4 | 5 | 6 | 7 | 8 | 9 | 10 | 11 | 12 | 13 | 14 | 15 | | | | | 16 | 17 | 21 | | 22 |
|---|---|---|---|---|---|---|---|---|---|---|---|---|---|---|---|---|---|---|---|---|---|---|---|---|
| | | Amphipat characteristics | | | | | | | | | | | | | | Amphipats | | | Aggregate compositions | | | | | |
| | | | Chains length, number, type | | | | | | | | | Headgroup type, leng | | | | 1 | 2 | 2.3 | 3 | | | weight | | |
| | | | | | | | | | | | # | 1 | 2 | 3 | 4 | Head | Head | Head | Head | | | #1/#2, 3 | | |
| | | L_{1,2} | DB₁ | n₁ | n₂ | L₃ | DB_{2,3} | n₃ | | | | Hea [symbol] | Head | Head | Head | | | | | | | w + w | | pH |
| | | | | | | | | | | | | | | EG/ | EG/ | | | | | | | | | |
| | Polyoxyethyleneglycol (pEG) | | | | | | | | Bond type | | | EG/n₁ | EG/n₂ | n₃ | n4 | | | | | | | g L⁻¹ | g L⁻¹ | |
| Emb. | direct chains attachment*, linear headgroup | | | | | | | | | | | | | | | | | | | “Average” mix | | | | |
| Nr. | Charged 3rd component | | | | | | | | #1 | #2 | #3 | #1 | | #2 | #3 | | | | | #2 | #3 | | | |
| 108 | Sulphate-EG, | C | 12 | 1 | 1 | 12 | | 1 | ET | ET | ET | 4.0 | 4.3 | 4 | 6 | Brij 30 | Av [symbol] | Brij 30 | EMAL 270 | 0.85 | 0.15 | 89.87 | 10.1 | 7.5 |
| | lauryl ether | | | | | | | | | | | | | | | | | | | | | | | |
| 109 | Sulphate, lauryl | C | 12 | 1 | 1 | 12 | | 1 | ET | ET | ET | 4.0 | 4.0 | 4 | 4 | Brij 30 | Av [symbol] | Brij 30 | WITCOLATE | 0.90 | 0.10 | 90.24 | 9.8 | 7.5 |
| | | | | | | | | | | | | | | | | | | | WA [symbol] | | | | | |
| 110 | Sulphate, hexadecyl | C | 18:1 | 1 | 1 | 16 | :1 | 1 | ET | ET | ET | 2.0 | [symbol] | 10.0 | 7.0 | Brij 93 | Av [symbol] | Brij 97 | | 0.67 | 0.33 | 73.42 | 26.6 | 7.5 |
| 111 | Phosphate, dicetyl | C | 18:1 | 1 | 2 | 16 | :1 | 2 | ET | ET | ET | 2.0 | [symbol] | 10.0 | 4.0 | Brij 93 | Av [symbol] | Brij 97 | | 0.83 | 0.17 | 72.39 | 27.6 | 7.5 |
| 112 | Phosphate, oleyl | C | 18:1 | 1 | 1 | 18 | :1 | 1 | ET | ET | ES | 2.0 | [symbol] | 10.0 | 4.0 | Brij 93 | Av [symbol] | Brij 97 | ILCO PHOS 208 | 0.83 | 0.17 | 73.44 | 26.56 | 7.5 |
| 113 | Phosphate, oleyl | C | 18:1 | 1 | 1 | 18 | :1 | 1 | ET | ET | ES | 2.0 | [symbol] | 10.0 | 4.0 | Brij 93 | Av [symbol] | Brij 97 | ILCO PHOS 208 | 0.92 | 0.08 | 72.51 | 27.5 | 7.5 |
| 114 | Phosphate-EG5, oleyl | C | 18:1 | 1 | 1 | 18 | :1 | 1 | ET | ET | ES | 2.0 | [symbol] | 10.0 | 5.0 | Brij 93 | Av [symbol] | Brij 97 | ILCO PHOS 215 | 0.80 | 0.20 | 74.00 | 26.0 | 7.5 |
| 115 | Sarcosinate, oleoyl | C | 18:1 | 1 | 1 | 18 | :1 | 1 | ET | ET | ES | 2.0 | [symbol] | 10.0 | 3.0 | Brij 93 | Av [symbol] | Brij 97 | Crodasinic | 0.86 | 0.14 | 52.08 | 47.9 | 7.5 |
| | | | | | | | | | | | | | | | | | | | O Oleo [symbol] | | | | | |
| 116 | Sulphonate, | C | 18:1 | 1 | 1 | 12-ben | | 1 | ET | ET | ET | 2.0 | [symbol] | 10.0 | 3.5 | Brij 93 | Av [symbol] | Brij 97 | WITCONATE | 0.85 | 0.15 | 81.48 | 18.5 | 7.5 |
| | dodecylbenzen | | | | | | | | | | | | | | | | | | 122 [symbol] | | | | | |
| | indirect linear chains attachment | | | | | | | | | | | | | | | | | | | | | | | |
| | sorbitan, branched headgroup; (stochastic) “anchor” chains distribution | | | | | | | | | | | | | | | | | | | | | | | |
| 117 | Phosphate, dicetyl | C | 18:1 | 1 | 1 | 16 | :1 | 2 | SES | SES | ES | 0.0 | [symbol] | 20.0 | 0.5 | Span 80 | Av [symbol] | Tween 80 | | 0.90 | 0.10 | 18.52 | 81.5 | 7.5 |
| 118 | Phosphate, cetyl | C | 18:1 | 1 | 1 | 16 | :1 | 1 | SES | SES | ES | 0.0 | [symbol] | 20.0 | 5.0 | Span 80 | Av [symbol] | Tween 80 | Crodafos MCA | 0.80 | 0.20 | 20.15 | 79.9 | 7.5 |
| 119 | Phosphate, oleyl | C | 18:1 | 1 | 1 | 18 | :1 | 1 | SES | SES | ES | 5.0 | 17.0 | 20.0 | 5.0 | Tween 81 | Av [symbol] | Tween 80 | ILCO PHOS 208 | 0.80 | 0.20 | 27.50 | 72.5 | 7.5 |
| 120 | Phosphatidylglycerol, | C | 18:1 | 1 | 1 | mix [symbol] | | 2 | SES | SES | ES | 0.0 | 18.0 | 20.0 | 0.5 | Tween 81 | Av [symbol] | Tween 80 | SPG | 0.90 | 0.10 | 25.16 | 74.8 | 7.5 |
| | dialke [symbol] | | | | | | | | | | | | | | | | | | | | | | | |
| 121 | Dimethylammonium, | C | 18:1 | 1 | 1 | 16 | :1 | 2 | SES | SES | A | 0.0 | 18.0 | 20.0 | 0.5 | Tween 81 | | Tween 80 | Aliquat 206 | 0.90 | 0.10 | 25.64 | 74.4 | 7.5 |
| | dihexa [symbol] | | | | | | | | | | | | | | | | | | | | | | | |
| | Polyglyceryl (pG) | | | | | | | | | | | G | | EG | | | | | | | | | | |
| | multiple “anchor” chain attachment options/(stochastic) distribution | | | | | | | | | | | | | | | | | | | | | | | |
| 122 | Phosphate-EG3, oleyl | C | 18:1 | 1 | 1 | | :1 | 2 | ES-G | SES | ES | 2.0 | 4.0 | 20.0 | 0.6 | [symbol] | Av [symbol] | Tween 80 | Crodafos N3A | 0.18 | 0.82 | 29.29 | 70.7 | 7.5 |

| | | | |
|---|---|---|---|
| Phosphatides (charged 1st component blend) | P-head^{c} | COO [symbol] | EG |

| 123 | PC/PG, dialkenoyl*** | C | 18:2 | 2 | 1 | mix [symbol] | | 1 | ET | ET | ES | 0.5 | [symbol] | [symbol] | 10 | SPC | Av [symbol] | Brij 93 | Brij 97 | 0.38 | 0.63 | 35.92 | 64.1 | 7.5 |
|---|---|---|---|---|---|---|---|---|---|---|---|---|---|---|---|---|---|---|---|---|---|---|---|---|
| 124 | PC/PG, dialkenoyl*** | C | 18:2 | 2 | 1 | mix [symbol] | | 1 | SES | SES | ES | 0.5 | [symbol] | 20.0 | 0 | SPC | Av [symbol] | Tween 80 | Span 80 | 0.40 | 0.60 | 29.27 | 70.7 | 7.5 |
| 125 | Lyso-PC/oleate ~1/1 | C | 18:2 | 1 | 1 | 18 | :2 | 1 | ES | ET | ES | 1 | [symbol] | 20.0 | 0.5 | lyso-SPC | Ol [symbol] | Brij 98 | | 1.00 | 0.25 | 48.11 | 51.9 | 8.5 |

| | Carboxylate (charged 2nd component) | | | | | | | | | | | | | | |
|---|---|---|---|---|---|---|---|---|---|---|---|---|---|---|---|
| 126 | Oleic acid/oleate**** | C | 18:1 | 1 | 1 | 18 | :1 | ES | ES | 1 | 1.0 | 0.0 | 50.00 | 50.0 | 8.5 |
| 127 | Linoleic acid/ | C | 18:2 | 1 | 1 | 18 | :2 | ES | ES | 1 | 1.0 | 0.0 | 50.00 | 50.0 | 8.5 |
| | linoleate**** | | | | | | | | | | | | | | |
| 128 | Linolenic acid/ | C | 18:3 | 1 | 1 | 18 | :3 | ES | ES | 1 | 1.0 | 0.0 | 50.00 | 50.0 | 8.5 |
| | linolenate*** | | | | | | | | | | | | | | |

*ET = ether; ES = ester; SES = sorbitan-ester;
A = amine
^{c}MW for di-chain phosphatidylcholines (PC/SPC) is given for the prevailing dihydrate
**C..i = iso-branching; L'gth = L = chain-length
***PC/PG = 9/1 phosphatidylcholine/phosphatidylglycerol mixture [symbol]
indicates data missing or illegible when filed

Table 3A.
Non-exclusive listing of excipients recommended for inclusion into preparations of the
invention (PART A)

| | | | | | | Embod. Nr.: | | | | | |
|---|---|---|---|---|---|---|---|---|---|---|---|
| | | | | | | | 17; 18; 35; | | 16; 34; | | |
| | | | | | | 17; 18; | 41; 48; 56; | 17; 18; | 38; 56; | 17; 18; | 17; 18; |
| | | | | | | 35; 41; | 59; 94; 97; | 35; 41; | 54; 97; | 35; 41; | 35; 41; |
| | | | | | | 48; 56; | 98; 111; | 48; 56; | 111; | 48; 56; | 48; 56; |
| | | | | | | 59; 94; | 114; 115; | 59; 94; | 114; | 59; 94; | 59; 94; |
| | | | | | | 97; 98 | 122, 123 | 97; 98 | 123 | 97; 98 | 97; 98 |
| Nrs. | | | w [% rel.] | [mM] | log P | Concentration used [g L−1] | | | | | |
| | | to aggregate m. | | | | | | | | | |
| | Microbicide | | | | | | | | | | |
| 1 | Acetic acid | 0.1-0.5 | | 10-66* | −0.17 | | | | | | |
| 2 | Benzoic acid | 0.05-0.5 | 0.05-0.5 | | 1.74 | | | | | | |
| 3 | Dehydroacetic acid | 0.2-1.0 | | 10-75* | −0.05 | | | | | | |
| 4 | Formic acid | 0.1-0.5 | | 20-100* | −0.48 | | | | | | |
| 5 | Propionic acid | 0.2 | 0.2 | 20-100 | 0.33 | | | | | | |
| 6 | Sorbic acid | | 0.025-0.1 | (5-25) | 1.38 | | | | | | |
| 7 | 2-(hydroxymethylamino)acetic | 0.1-0.8** | | 10-80* | −2.2 | | | | | | |
| | adic; N-(hydroxymethyl) | | | | | | | | | | |
| 8 | Dimethoxane; (2,6-dimethyl- | | 0.05-0.3 | | 0.87 | | | | | | |
| | 1,3-dioxan-4-yl) acetate | | | | | | | | | | |
| 9 | Benzalkonium | | 0.01-0.2 | | 3.67 | | | | | | |
| 10 | Benzethonium | | 0.01-0.1 | | | | | | | | |
| 10 | 5-Bromo-5-nitro-1,3- | 25-150 m [symbol] | 0.05-0.3 | (0.05-0.3) | 0.44 | 1.000 | | 0.05-0.3 | | | |
| | dioxane, bronidox | | | | | | | | | | |
| 11 | 2-bromo-2-nitropropane-1,3- | 0.05-0.3 | | 25-150 | −0.33 | | | | | | |
| | diol, bronopol | | | | | | | | | | |
| 12 | Diazodinyl Urea (Germall II) | 0.2-0.5 | | 75-175 | −3.2 | | | | | | |
| 13 | 1,3-Dimethylol-5,5- | | 0.15-0.4 | (7.5-30) | 0.6 | | | | | | |
| | dimethylhydantoin; glydant | | | | | | | | | | |
| 14 | Methyl 4-hydroxybenzoate; | | 0.1-5 | | 1.72 | | 1.125 | | | | 0.450 |
| | methylparabene | | | | | | | | | | |
| 15 | Ethyl 4-hydroxybenzoate; | | 0.1-3 | | 2.16 | | | | | | |
| | ethylparabene | | | | | | | | | | |
| 16 | Propyl 4-hydroxybenzoate; | | 0.1-1.5 | | 2.63 | | 1.125 | | | | 0.450 |
| | propylparabene | | | | | | | | | | |
| 17 | Butyl 4-hydroxybenzoate; | | 0.1-3 | | 3.07 | | 0.250 | | | | 0.100 |
| | butylparabene | | | | | | | | | | |
| 18 | Methylisothiazolone; 2-methyl- | 0.05-0.1 | | 50-100 | −0.06 | | | 0.500 | 0.075 | | 0.008 |
| | 1,2-thiazol-3-one | | | | | | | | | | |
| 19 | Methylchloroisothiazolinone | | 0.05-0.1 | | 0.74 | | | | | 0.500 | |
| 20 | Benzisothiazolone, 1,2- | | 0.05-0.25 | | 1.08 | | | | | | |
| | benzothiazol-3-one | | | | | | | | | | |
| 21 | Phenoxyethanol | | 0.1-5 | | 1.26 | | | | | | |
| 22 | Phenylethanol | | 0.1-1 | | 1.61 | | | | | | |
| 23 | Quaternium-15; | 0.05-0.3 | | 20-125 | [symbol] | | | | | | |
| | Dowicil ® 200 | | | | | | | | | | |
| 24 | Nipaguard ® CMB or DCB or PBI | 0.03-0.3 | | 0.03-0.3** | | | | | | | |
| 25 | Euxyl ® K 145 | 0.05-0.15 | | 0.05-0.15** | | | | | | | |
| | Co-solvent | | | | | | | | | | |
| 26 | Benzyl alcohol | | 0-0.5 | | 1.23 | | | | 5.000 | | |
| 27 | Ethanol | | 0-15 | 0-15** | −0.05 | | | | | | |
| 28 | Propanediol | | 0-15 | 0-10** | −0.71 | | | | | | |

Table 3B.
Non-exclusive listing of excipients recommended for inclusion into preparations of the
invention (PART B)

| Embod. Nr.: | | | | | |
|---|---|---|---|---|---|
| | 17; 18; 35; | | 16; 34; | | |
| 17; 18; | 41; 48; 56; | 17; 18; | 38; 56; | 17; 18; | 17; 18; |
| 35; 41; | 59; 94; 97; | 35; 41; | 54; 97; | 35; 41; | 35; 41; |
| 48; 56; | 98; 111; | 48; 56; | 111; | 48; 56; | 48; 56; |
| 59; 94; | 114; 115; | 59; 94; | 114; | 59; 94; | 59; 94; |
| 97; 98 | 122, 123 | 97; 98 | 123 | 97; 98 | 97; 98 |

| Nrs. | | w [% rel.] | [mM] | log P | Concentrations used [g L−1] |
|---|---|---|---|---|---|
| | | to aggregate m. | | | |
| | Humectant | | | | |
| 29 | Glycerol | | 0-15** | | |
| 30 | Urea | | 0-15** | | |
| 31 | Triacetin, glycerin triacetate | | 2.5** | | |
| 32 | Tetramethylurea | | 0-10** | | |
| 33 | Hyaluronic acid | | 0-5** | | |
| | Antioxidant | | | | |
| 34 | 2,3,5-trimethylbenzene-1,4-diol | 0.1-10 | | 2.12 | |
| 35 | Butylated hydroxyanisole, BHA | 0.1-8 | | 3.13 | |
| 36 | 4-hexoxy-2,3,6-trimethylphenol, HTHQ | 0.1-8 | | 4.81 | |
| 37 | Butylated hydroxytoluene, BHT | 0.1-4 | | 5.06 | |
| 38 | 6-isopropyl-m-cresol; thymol | 0.1-1 | | 3.21 | |
| 39 | Alkyl-ascorbate | 0.1-1 | | | |
| 40 | Tocopherol | 0-5 | | 9.76 | |
| 41 | Tocopherol-PEG | 0-5 | | | |
| 42 | Ascorbic acid | | 0-2** | | |
| 43 | Metabisulphite | | 1-5** | | |
| 44 | Bisulphite | | 1-5** | | |
| 45 | Thiourea | | 1-10** | −0.96 | |
| 46 | 3-sulfanylpropane-1,2-diol | | 1-20** | −0.57 | |
| | Chellator (secondary “antioxidant”) | | | | |
| 47 | Br-Benzyl-teta | | 1-8 | −8 | |
| 48 | Edetic acid (EDTA | | 1-10 | | |
| 49 | Egtazic acid (EGTA) | | 1-10 | | |
| 50 | Ethyleneglycol-bis-N,N′-tetraacetic acid | | 1-10 | | |
| 51 | Deferoxamine | | 0.1-5 | | |
| | Fragrance | | | | |
| 52 | Linalool | | 0.5-10 | | |
| 53 | Myrcene/myrcenol | | 0.25-10 | | |
| 54 | Lavender oil | | 0.25-10 | | |
| 55 | Menthol | | 0.5-10 | | |
| 56 | cis-3-Hexen-1-ol | | 0.5-10 | | |
| 57 | Geraniol | | 0.5-10 | | |

Table 3C.
Non-exclusive listing of excipients recommended for inclusion into preparations of the
invention (PART C)

| | | | | | Embod. Nr.: | | | | | | | |
|---|---|---|---|---|---|---|---|---|---|---|---|---|
| | | | | | 17; 18; | 18; 35; | 16; 34; | | | | | |
| | | | | | 35; 41; | 41; 48; | 38; 56; | 18; 35; | 17; 35; | | 17; 35; | 40; |
| | | | | | 48; 56; | 56; 59; | 54; 97; | 41; 48; | 41; 48; | 16; 34; | 41; 48; | 56; |
| | | | | | 59; 94; | 93; 94; | 111; 114; | 56; 59; | 59; 93; | 38; 56; | 59; 93; | 93 |
| Nrs. | | | w [% rel.] | [mM] | 97; 98 | 98 | 115; 122 | 97; 98 | 98 | 54; 97 | 98 | |
| | | to aggregate m. | | | | | | | | | | |
| | Microbicide | | | | | | | | | | | |
| 1 | Acetic acid | 0.1-0.5 | | 10-66* | | | | | | | | |
| 2 | Benzoic acid | 0.05-0.5 | 0.05-0.5 | | | | | | | | | |
| 3 | Dehydroacetic acid | 0.2-1.0 | | 10-75* | | | | | | | | |
| 4 | Formic acid | 0.1-0.5 | | 20-100* | | | | | | | | |
| 5 | Propionic acid | 0.2 | 0.2 | 20-100 | | | | | | | | |
| 6 | Sorbic acid | | 0.025-0.1 | ( [symbol] ) | | | | | | | | |
| 7 | 2- | 0.1-0.8** | | 10-80* | | | | | 5.000 | | | |
| | (hydroxymethyl- | | | | | | | | | | | |
| | amino)acetic | | | | | | | | | | | |
| | adic; N-(hydroxymethyl) | | | | | | | | | | | |
| 8 | Dimethoxane; | | 0.05-0.3 | | | | | | | | | |
| | (2,6-dimethyl-1,3-dioxan- | | | | | | | | | | | |
| | 4-yl) acetate | | | | | | | | | | | |
| 9 | Benzalkonium | | 0.01-0.2 | | | | | | | | | |
| 10 | Benzethonium | | 0.01-0.1 | | | | | | | | | |
| 10 | 5-Bromo-5-nitro-1,3- | 25-150 | 0.05-0.3 | (0.05-0.3) | | | | | | | | |
| | dioxane, bronidox | m [symbol] | | | | | | | | | | |
| 11 | 2-bromo-2-nitropropane- | 0.05-0.3 | | 25-150 | | | | | | | | 1.00 |
| | 1,3-diol, bronopol | | | | | | | | | | | |
| 12 | Diazodinyl Urea | 0.2-0.5 | | 75-175 | 5.000 | 2.000 | | | | | | |
| | (Germall II) | | | | | | | | | | | |
| 13 | 1,3-Dimethylol-5,5- | | 0.15-0.4 | (7.5-30) | | | | | | | 1.500 | |
| | dimethylhydantoin; glydant | | | | | | | | | | | |
| 14 | Methyl 4-hydroxybenzoate; | | 0.1-5 | | | 0.450 | | | | | | |
| | methylparabene | | | | | | | | | | | |
| 15 | Ethyl 4-hydroxybenzoate; | | 0.1-3 | | | | | | | | | |
| | ethylparabene | | | | | | | | | | | |
| 16 | Propyl 4-hydroxybenzoate; | | 0.1-1.5 | | | 0.450 | | | | | | |
| | propylparabene | | | | | | | | | | | |
| 17 | Butyl 4-hydroxybenzoate; | | 0.1-3 | | | 0.100 | | | | | | |
| | butylparabene | | | | | | | | | | | |
| 18 | Methylisothiazolone; | 0.05-0.1 | | 50-100 | | | | | | | | |
| | 2-methyl-1,2-thiazol-3-one | | | | | | | | | | | |
| 19 | Methylchloro- | | 0.05-0.1 | | | | | | | | | |
| | isothiazolinone | | | | | | | | | | | |
| 20 | Benzisothiazolone, | | 0.05-0.25 | | | | | | | | | |
| | 1,2-benzothiazol-3-one | | | | | | | | | | | |
| 21 | Phenoxyethanol | | 0.1-5 | | | | | | | 1.000 | | |
| 22 | Phenylethanol | | 0.1-1 | | | | | | | | | |
| 23 | Quaternium-15; | 0.05-0.3 | | 20-125 | | | 2.000 | | | | | |
| | Dowicil ® 200 | | | | | | | | | | | |
| 24 | Nipaguard ® CMB or | 0.03-0.3 | | 0.03-0.3** | | | | 0.015* | | | | |
| | DCB or PBI | | | | | | | | | | | |
| 25 | Euxyl ® K 145 | 0.05-0.15 | | 0.05-0.15** | | | | 1.500 | | | | |

| | | Parabenes total | | | 1.000 | |
|---|---|---|---|---|---|---|
| | Co-solvent | | | | | |
| 26 | Benzyl alcohol | | 0-0.5 | | | 5.000 |
| 27 | Ethanol | | 0-15 | 0-15** | | |
| 28 | Propanediol | | 0-15 | 0-10** | | |

Table 3D.
Non-exclusive listing of excipients recommended for inclusion into preparations of the
invention (PART D)

| | | | Embod. Nr.: | | | | | | | |
|---|---|---|---|---|---|---|---|---|---|---|
| | | | 17; 18; | 18; 35; | 16; 34; | | | | | |
| | | | 35; 41; | 41; 48; | 38; 56; | 18; 35; | 17; 35; | | 17; 35; | 40; |
| | | | 48; 56; | 56; 59; | 54; 97; | 41; 48; | 41; 48; | 16; 34; | 41; 48; | 56; |
| | | | 59; 94; | 93; 94; | 111; 114; | 56; 59; | 59; 93; | 38; 56; | 59; 93; | 93 |
| Nrs. | w [% rel.] | [mM] | 97; 98 | 98 | 115; 122 | 97; 98 | 98 | 54; 97 | 98 | |

| | | to aggregate m. | |
|---|---|---|---|
| | Humectant | | |
| 29 | Glycerol | | 0-15** |
| 30 | Urea | | 0-15** |
| 31 | Triacetin, glycerin triacetate | | 2.5** |
| 32 | Tetramethylurea | | 0-10** |
| 33 | Hyaluronic acid | | 0-5** |
| | Antioxidant | | |
| 34 | 2,3,5-trimethylbenzene-1,4-diol | 0.1-10 | |
| 35 | Butylated hydroxyanisole, BHA | 0.1-8 | |
| 36 | 4-hexoxy-2,3,6-trimethylphenol, HTHQ | 0.1-8 | |
| 37 | Butylated hydroxytoluene, BHT | 0.1-4 | |
| 38 | 6-isopropyl-m-cresol; thymol | 0.1-1 | |
| 39 | Alkyl-ascorbate | 0.1-1 | |
| 40 | Tocopherol | 0-5 | |
| 41 | Tocopherol-PEG | 0-5 | |
| 42 | Ascorbic acid | | 0-2** |
| 43 | Metabisulphite | | 1-5** |
| 44 | Bisulphite | | 1-5** |
| 45 | Thiourea | | 1-10** |
| 46 | 3-sulfanylpropane-1,2-diol | | 1-20** |
| | Chellator (secondary “antioxidant”) | | |
| 47 | Br-Benzyl-teta | | 1-8 |
| 48 | Edetic acid (EDTA | | 1-10 |
| 49 | Egtazic acid (EGTA) | | 1-10 |
| 50 | Ethyleneglycol-bis-N,N′-tetraacetic acid | | 1-10 |
| 51 | Deferoxamine | | 0.1-5 |
| | Fragrance | | |
| 52 | Linalool | | 0.5-10 |
| 53 | Myrcene/myrcenol | | 0.25-10 |
| 54 | Lavender oil | | 0.25-10 |
| 55 | Menthol | | 0.5-10 |
| 56 | cis-3-Hexen-1-ol | | 0.5-10 |
| 57 | Geraniol | | 0.5-10 |

Table 3E.
Non-exclusive listing of excipients recommended for inclusion into preparations of the
invention (PART E)

| | | | Embod. Nr.: | | | | | | |
|---|---|---|---|---|---|---|---|---|---|
| | | | | 16; 17; 18; | 16; 17; 18; | 18; | 35; | 35; | 16; 17; 18; 34; |
| | | | 17; 18; | 34; 35; 38; | 34; 35; 38; | 35; | 38; | 38; | 35; 38; 41; 48; |
| | | | 35; 41; | 41; 48; 54; | 41; 48; 54; | 41; | 48; | 48; | 54; 56; 59; 94; |
| | | | 48; 59; | 56; 59; 94; | 56; 59; 94; | 56; | 59; | 59; | 97; 98; 111; |
| Nrs. | w [% rel.] | [mM] | 93 | 97; 98 | 97; 98 | 59 | 93 | 93 | 122 |

| | | to aggregate m. | | | |
|---|---|---|---|---|---|
| | Microbicide | | | | |
| 1 | Acetic acid | 0.1-0.5 | | 10-66* | 4.000 |
| 2 | Benzoic acid | 0.05-0.5 | 0.05-0.5 | | |
| 3 | Dehydroacetic acid | 0.2-1.0 | | 10-75* | |
| 4 | Formic acid | 0.1-0.5 | | 20-100* | |
| 5 | Propionic acid | 0.2 | 0.2 | 20-100 | |
| 6 | Sorbic acid | | 0.025-0.1 | ( [symbol] ) | |
| 7 | 2-(hydroxymethylamino)acetic adic; | 0.1-0.8** | | 10-80* | |
| | N-(hydroxymethyl) | | | | |
| 8 | Dimethoxane; (2,6-dimethyl-1,3- | | 0.05-0.3 | | |
| | dioxan-4-yl) acetate | | | | |
| 9 | Benzalkonium | | 0.01-0.2 | | |
| 10 | Benzethonium | | 0.01-0.1 | | |
| 10 | 5-Bromo-5-nitro-1,3-dioxane, | 25-150 ml | 0.05-0.3 | (0.05-0.3) | |
| | bronidox | | | | |
| 11 | 2-bromo-2-nitropropane-1,3-diol, | 0.05-0.3 | | 25-150 | |
| | bronopol | | | | |
| 12 | Diazodinyl Urea (Germall II) | 0.2-0.5 | | 75-175 | |
| 13 | 1,3-Dimethylol-5,5- | | 0.15-0.4 | (7.5-30) | |
| | dimethylhydantoin; glydant | | | | |
| 14 | Methyl 4-hydroxybenzoate; | | 0.1-5 | | |
| | methylparabene | | | | |
| 15 | Ethyl 4-hydroxybenzoate; | | 0.1-3 | | |
| | ethylparabene | | | | |
| 16 | Propyl 4-hydroxybenzoate; | | 0.1-1.5 | | |
| | propylparabene | | | | |
| 17 | Butyl 4-hydroxybenzoate; | | 0.1-3 | | |
| | butylparabene | | | | |
| 18 | Methylisothiazolone; 2-methyl-1,2- | 0.05-0.1 | | 50-100 | |
| | thiazol-3-one | | | | |
| 19 | Methylchloroisothiazolinone | | 0.05-0.1 | | |
| 20 | Benzisothiazolone, 1,2- | | 0.05-0.25 | | |
| | benzothiazol-3-one | | | | |
| 21 | Phenoxyethanol | | 0.1-5 | | |
| 22 | Phenylethanol | | 0.1-1 | | |
| 23 | Quaternium-15; Dowicil ® 200 | 0.05-0.3 | | 20-125 | |
| 24 | Nipaguard ® CMB or DCB or PBI | 0.03-0.3 | | 0.03-0.3** | |
| 25 | Euxyl ® K 145 | 0.05-0.15 | | 0.05-0.15** | |

| | | Parabenes total | | |
|---|---|---|---|---|
| | Co-solvent | | | |
| 26 | Benzyl alcohol | | 0-0.5 | |
| 27 | Ethanol | | 0-15 | 0-15** |
| 28 | Propanediol | | 0-15 | 0-10** |

Table 3F.
Non-exclusive listing of excipients recommended for inclusion into preparations of the
invention (PART F)

| | | | | Embod. Nr.: | | | | | | |
|---|---|---|---|---|---|---|---|---|---|---|
| | | | | | 16; 17; 18; | 16; 17; 18; | 18; | 35; | 35; | 16; 17; 18; 34; |
| | | | | 17; 18; | 34; 35; 38; | 34; 35; 38; | 35; | 38; | 38; | 35; 38; 41; 48; |
| | | | | 35; 41; | 41; 48; 54; | 41; 48; 54; | 41; | 48; | 48; | 54; 56; 59; 94; |
| | | | | 48; 59; | 56; 59; 94; | 56; 59; 94; | 56; | 59; | 59; | 97; 98; 111; |
| Nrs. | | w [% rel.] | [mM] | 93 | 97; 98 | 97; 98 | 59 | 93 | 93 | 122 |
| | | to aggregate m. | | | | | | | | |
| | Humectant | | | | | | | | | |
| 29 | Glycerol | | 0-15** | | 5.00***^{:+} | 10.00*** | | | | |
| 30 | Urea | | 0-15** | | 5.00***^{:+} | 10.00*** | | | | |
| 31 | Triacetin, glycerin triacetate | | 2.5** | | | | 2.5 | | | |
| 32 | Tetramethylurea | | 0-10** | | | | | 5.0 | | |
| 33 | Hyaluronic acid | | 0-5** | | | | | | | |
| | Antioxidant | | | | | | | | | |
| 34 | 2,3,5-trimethylbenzene-1,4-diol | 0.1-10 | | | | | | | 0.3 | |
| 35 | Butylated hydroxyanisole, BHA | 0.1-8 | | | | | | | | 0.250 |
| 36 | 4-hexoxy-2,3,6-trimethylphenol, HTHQ | 0.1-8 | | | | | | | | |
| 37 | Butylated hydroxytoluene, BHT | 0.1-4 | | | | | | | | |
| 38 | 6-isopropyl-m-cresol; thymol | 0.1-1 | | | | | | | | |
| 39 | Alkyl-ascorbate | 0.1-1 | | | | | | | | |
| 40 | Tocopherol | 0-5 | | | | | | | | |
| 41 | Tocopherol-PEG | 0-5 | | | | | | | | |
| 42 | Ascorbic acid | | 0-2** | | | | | | | |
| 43 | Metabisulphite | | 1-5** | | | | | | | |
| 44 | Bisulphite | | 1-5** | | | | | | | |
| 45 | Thiourea | | 1-10** | | | | | | | |
| 46 | 3-sulfanylpropane-1,2-diol | | 1-20** | | | | | | | |
| | Chellator (secondary “antioxidant”) | | | | | | | | | |
| 47 | Br-Benzyl-teta | | 1-8 | | | | | | | |
| 48 | Edetic acid (EDTA | | 1-10 | | | | | | | |
| 49 | Egtazic acid (EGTA) | | 1-10 | | | | | | | |
| 50 | Ethyleneglycol-bis-N,N′-tetraacetic acid | | 1-10 | | | | | | | |
| 51 | Deferoxamine | | 0.1-5 | | | | | | | |
| | Fragrance | | | | | | | | | |
| 52 | Linalool | | 0.5-10 | | | | | | | |
| 53 | Myrcene/myrcenol | | 0.25-10 | | | | | | | |
| 54 | Lavender oil | | 0.25-10 | | | | | | | |
| 55 | Menthol | | 0.5-10 | | | | | | | |
| 56 | cis-3-Hexen-1-ol | | 0.5-10 | | | | | | | |
| 57 | Geraniol | | 0.5-10 | | | | | | | |

Table 3G.
Non-exclusive listing of excipients recommended for inclusion into preparations of the
invention (PART G)

| | | | Embod. Nr.: | | | |
|---|---|---|---|---|---|---|
| | | | 17; | | | |
| | | | 35; | 16; 17; 18; 34; | 16; 17; 18; | 16; 17; 18; |
| | | | 41; | 35; 38; 41; 48; | 34; 35; 38; | 34; 35; 38; |
| | | | 48; | 54; 56; 59; 94; | 41; 48; 54; | 41; 48; 54; |
| | | | 59; | 97; 98; 114; | 56; 59; 94; | 56; 59; 94; |
| Nrs. | w [% rel.] | [mM] | 93 | 123 | 97; 98 | 97; 98 |

| | | to aggregate m. | | |
|---|---|---|---|---|
| | Microbicide | | | |
| 1 | Acetic acid | 0.1-0.5 | | 10-66* |
| 2 | Benzoic acid | 0.05-0.5 | 0.05-0.5 | |
| 3 | Dehydroacetic acid | 0.2-1.0 | | 10-75* |
| 4 | Formic acid | 0.1-0.5 | | 20-100* |
| 5 | Propionic acid | 0.2 | 0.2 | 20-100 |
| 6 | Sorbic acid | | 0.025-0.1 | (5-25) |
| 7 | 2-(hydroxymethylamino)acetic adic; N-(hydroxymethyl) | 0.1-0.8** | | 10-80* |
| 8 | Dimethoxane; (2,6-dimethyl-1,3-dioxan-4-yl) acetate | | 0.05-0.3 | |
| 9 | Benzalkonium | | 0.01-0.2 | |
| 10 | Benzethonium | | 0.01-0.1 | |
| 10 | 5-Bromo-5-nitro-1,3-dioxane, bronidox | 25-150 ml | 0.05-0.3 | (0.05-0.3) |
| 11 | 2-bromo-2-nitropropane-1,3-diol, bronopol | 0.05-0.3 | | 25-150 |
| 12 | Diazodinyl Urea (Germall II) | 0.2-0.5 | | 75-175 |
| 13 | 1,3-Dimethylol-5,5-dimethylhydantoin; glydant | | 0.15-0.4 | (7.5-30) |
| 14 | Methyl 4-hydroxybenzoate; methylparabene | | 0.1-5 | |
| 15 | Ethyl 4-hydroxybenzoate; ethylparabene | | 0.1-3 | |
| 16 | Propyl 4-hydroxybenzoate; propylparabene | | 0.1-1.5 | |
| 17 | Butyl 4-hydroxybenzoate; butylparabene | | 0.1-3 | |
| 18 | Methylisothiazolone; 2-methyl-1,2-thiazol-3-one | 0.05-0.1 | | 50-100 |
| 19 | Methylchloroisothiazolinone | | 0.05-0.1 | |
| 20 | Benzisothiazolone, 1,2-benzothiazol-3-one | | 0.05-0.25 | |
| 21 | Phenoxyethanol | | 0.1-5 | |
| 22 | Phenylethanol | | 0.1-1 | |
| 23 | Quaternium-15; Dowicil ® 200 | 0.05-0.3 | | 20-125 |
| 24 | Nipaguard ® CMB or DCB or PBI | 0.03-0.3 | | 0.03-0.3** |
| 25 | Euxyl ® K 145 | 0.05-0.15 | | 0.05-0.15** |
| | | Parabenes total | | |
| | Co-solvent | | | |
| 26 | Benzyl alcohol | | 0-0.5 | |
| 27 | Ethanol | | 0-15 | 0-15** |
| 28 | Propanediol | | 0-15 | 0-10** |

Table 3H.
Non-exclusive listing of excipients recommended for inclusion into preparations of the
invention (PART H)

| | | | | Embod. Nr.: | | | | |
|---|---|---|---|---|---|---|---|---|
| | | | | | | 16; 17; 18; 34; | 16; 17; 18; | 16; 17; 18; |
| | | | | | | 35; 38; 41; 48; | 34; 35; 38; | 34; 35; 38; |
| | | | | | | 54; 56; 59; 94; | 41; 48; 54; | 41; 48; 54; |
| | | | | 17; 35; 41; | | 97; 98; 114; | 56; 59; 94; | 56; 59; 94; |
| Nrs. | | w [% rel.] | [mM] | 48; 59; 93 | | 123 | 97; 98 | 97; 98 |
| | | to aggregate m. | | | | | | |
| | Humectant | | | | | | | |
| 29 | Glycerol | | 0-15** | | | | | |
| 30 | Urea | | 0-15** | | | | | |
| 31 | Triacetin, glycerin triacetate | | 2.5** | | | | | |
| 32 | Tetramethylurea | | 0-10** | | | | | |
| 33 | Hyaluronic acid | | 0-5** | | | | | |
| | Antioxidant | | | | | | | |
| 34 | 2,3,5-trimethylbenzene-1,4-diol | 0.1-10 | | | | | | |
| 35 | Butylated hydroxyanisole, BHA | 0.1-8 | | | | | | |
| 36 | 4-hexoxy-2,3,6-trimethylphenol, HTHQ | 0.1-8 | | | | | | |
| 37 | Butylated hydroxytoluene, BHT | 0.1-4 | | 0.200 | | | | |
| 38 | 6-isopropyl-m-cresol; thymol | 0.1-1 | | | | | | |
| 39 | Alkyl-ascorbate | 0.1-1 | | | | | | |
| 40 | Tocopherol | 0-5 | | | | | | |
| 41 | Tocopherol-PEG | 0-5 | | | | | | |
| 42 | Ascorbic acid | | 0-2** | | | | | |
| 43 | Metabisulphite | | 1-5** | | | | | |
| 44 | Bisulphite | | 1-5** | | | | | |
| 45 | Thiourea | | 1-10** | | | | | |
| 46 | 3-sulfanylpropane-1,2-diol | | 1-20** | | | | | |
| | Chellator (secondary “antioxidant”) | | | | | | | |
| 47 | Br-Benzyl-teta | | 1-8 | | | | | |
| 48 | Edetic acid (EDTA | | 1-10 | | 3**** | | | |
| 49 | Egtazic acid (EGTA) | | 1-10 | | | | | |
| 50 | Ethyleneglycol-bis-N,N′-tetraacetic acid | | 1-10 | | | | | |
| 51 | Deferoxamine | | 0.1-5 | | | | | |
| | Fragrance | | | | | | | |
| 52 | Linalool | | 0.5-10 | | | 1.000 | | |
| 53 | Myrcene/myrcenol | | 0.25-10 | | | | 0.500 | |
| 54 | Lavender oil | | 0.25-10 | | | | | 0.250 |
| 55 | Menthol | | 0.5-10 | | | | | |
| 56 | cis-3-Hexen-1-ol | | 0.5-10 | | | | | |
| 57 | Geraniol | | 0.5-10 | | | | | |

[symbol]

indicates data missing or illegible when filed

### 6.5 Biological Examples

The persistence of a hydrated suspension of highly adaptable vesicles on the skin was tested by measuring the difference between the skin surface temperature at a treated area and an untreated area. The results are given as a function of time after a non-occlusive application of a representative aggregate formulation of the invention in Table 4. After a short drying period (−15≦t/min≦0), during which the excess water evaporates (but vesicle hydration is maintained), a measurable temperature difference remains, consistent with a longer preservation of aggregates on skin surface.

TABLE 4
The cutaneous temperature difference:
ΔT = T_{treated site} − T_{untreated site} (n = 6).

| Time/min | ΔT/° C. |
|---|---|
| −15 | −2.50 |
| 0 | −0.21 |
| 30 | −0.11 |
| 60 | −0.15 |

To further biological characterisation of various adaptable vesicle preparations of the invention, their local anti-inflammatory activity was compared with several positive and negative controls, relying on a mustard-oil challenge test (cf. Cevc, 2012, op. cit.). One active preparation was topical Voltaren® Emulgel® (Novartis, “Volt. Em.”) containing the NSAID diclofenac (1.16%) as a diethylamine salt. Another positive control was a semisolid suspension containing 2.29% ketoprofen in ultra deformable aggregates within a gel (“KTAG”) having an overall composition similar to known compositions. Yet another control was hydrocortisone in an ethanol-based solution (Ebenol® Spray, Strathmann). Untreated but challenged sites were negative controls. A suspension with a similar composition was the ketoprofen-containing positive control, but without the drug (that would make the aggregates more adaptable and making vesicles more similar to conventional liposomes) was used as another control. Unless stated otherwise, all tested formulations were applied 1 h after inflammation induction (“post-treatment”) to exclude false positive results caused by irritant binding to aggregates or any of their components.

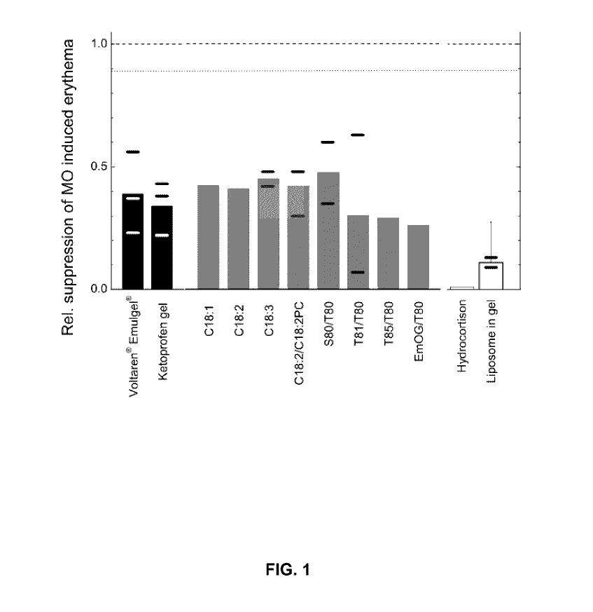

The results obtained with several simple preparations (fluid suspensions) and several preparations supplemented with select additives derivable from Table 3, including but not limited to a thickener (semisolid suspensions) are illustrated in FIG. 1. These results cover formulations with comparable headgroups (fatty acid/fatty soap mixtures) and differing degrees of chain unsaturation (i.e., 1, 2, or 3 double bonds per chain, corresponding to embodiments 126=C18:1, 127=C18:2, 128=C18:3 of Table 2) and similar chains with different headgroups (127=C18:2, 125=C18:2/C18:2 PC; 126=C18:1, 34=S80/T80, 39=T81/80, 43=T85/80, 56=EmOG/T80). Similar results were obtained in confirming experiments carried out using the T80=Tween 80-containing Example 34 with 5 wt.-% ethanol or with the suspensions of Example 39 where dehydroacetic acid replaces phosphate buffer (both modifications required around 30% T80 content reduction, however, since they both boosted vesicle adaptability, as shown by the resulting shorter vesicularisation times)(data not shown).

Collectively, the results illustrated in FIG. 1 demonstrate that various tested formulations containing adaptable aggregates as specified in Tables 1 to 3 suppress local inflammation more effectively than less adaptable aggregates, such as therapeutically ineffective liposomes. The results in FIG. 1 further show that the observed effects are distinct from the putative phospholipid-dependent anti-inflammatory action already known in the art. Moreover, the “Area Under the Curve” calculated from the time-course of erythema (AUC) even suggests that all the tested preparations containing adaptable vesicles are nearly as therapeutically effective as topical NSAID (ketoprofen or diclofenac) treatments, since the drug-free preparations similarly reduced the overall inflammatory response by about 40%, independent of precise composition of the highly adaptable vesicles of the invention. This result suggests that the observed beneficial clinical result is not due to any particular formulation component alone. Rather, the advantageous result appears to originate in the collective physical properties of the aggregates made according to this invention.

Another observation, made about 24 h post irritation, is that those sites characterised with a lower level of erythema were also less hyperalgesic (i.e. less sensitive to local irritation by heat or rubbing). Owing to the qualitative nature of these observations, these results are not detailed further herein.

A different type of assay tested the “pretreatment efficiency” of the above-described three different aggregate preparations, where lysophospholipid-fatty acid/soap mixture (i.e. Example 125) was again compared with drug-free liposomes. The observed difference of about 20% between the pretreatment effect of adaptable aggregates (C18:2 PC: AAUC: 48%) and of liposome-like vesicles (AAUC: 28%) is approximately two times greater than the AUC variability of around 10%, as determined by a separate data analysis. These observations confirm that the compositions of the invention effectively suppress local inflammation in a manner that is superior to the drug-free preparations containing known phospholipid-based vesicles.

Additional confirmation of local irritation/inflammation suppression effect using the inventive formulations was made using different in vivo method, i.e. the UVB-induced skin erythema and hyperalgesia test (Rother & Rother, 2011 Pain Res 4: 357). Consistent with the results shown in FIG. 1, this latter test, using physical rather than chemical skin irritation, confirmed the suppression of mild erythema and hyperalgesia, i.e. a therapeutic effect, of Example 34 (=S80/T80, but with added fragrance, microbicide, humectant and thickening agent of Table 3) to be similar to Voltaren® Emulgel® (Novartis). In contrast, locally applied hydrocortisone solution was practically indistinguishable from an untreated control. This confirmed, first, therapeutic efficacy of non-phospholipid adaptable vesicles in vivo that was unknown from scientific literature before, and, second, compatibility of adaptable vesicle technology described herein with the additives identified in Table 3.

Further evidence showing therapeutic anti-inflammatory and pain-suppressing effects of various formulations comprising NSAID-free highly adaptable aggregates of the invention is derived from an informal study involving several osteoarthritic patients.

One treated subject (allergic to, thus untreatable by, NSAIDs) has suffered from chronic pain associated with osteoarthritis, especially in the hands. This patient underwent a treatment regimen of a1-2 times daily application of a preparation corresponding to Example 34 according to the invention, which included several key additives depicted in Table 3 (i.e. a thickener, microbicide, fragrance, humectant). The applied dose per area also followed the guidance herein. Clinical symptoms of the disease following application thereafter improved significantly and the swelling decreased.

A second subjected suffered from a less chronic osteoarthritis manifesting itself in occasional localised, mild, flares. This patient treated one such flare in the thumb region using a preparation of the present invention (Example 43+thickener, microbicide, fragrance, humectant). The disease-associated pain improved after few days of treatment, declined once treatment was discontinued, but improved once this therapy was resumed.

Additional evidence supporting the therapeutic aspects of the invention involves a subject who experienced pain in one shoulder following unusually strong physical activity. Application of the inventive preparations (Example 34, twice daily, ca. 10 mg total amphipat mass per cm²) clearly demonstrated an improvement in these indications within 3-4 days from the first application of the aggregate composition.

1. Composition for use in diminishing inflammation and/or pain in a mammal comprising adaptable vesicular aggregates, wherein the composition is pharmacological agent-free and preferably phospholipid-free.

2. The composition for the use of claim 1,
wherein the relative molar concentrations of phospholipids, if any, compared with the concentration of all other aggregate-forming amphipats in the composition taken together is below 66 mol-%.

3. The composition for the use of claim 1,
wherein the vesicularisation time of said aggregates in a polar fluid is at least 5-times shorter than the vesicularisation time of comparably suspended vesicles containing>90% pure soybean phosphatidylcholine liposomes.

4. The composition for the use of claim 1,
wherein said aggregates have an average diameter of between 20 nm and 1 μm nm), and
wherein said amphipats occupy an average area per fluid chain in the bilayer of between about 0.35 nm² and about 0.55 nm², preferably of 0.43±0.05 nm².

5. The composition for the use of claim 1, wherein said aggregates comprise:
at least one amphipat characterised by a Hydrophilic-Lipophilic-Balance (HLB) number in the range of about 13.5>HLB>6.5, preferably in the range of about 12.5>HLB>7.5, and
wherein dispersion of the composition in a polar fluid yields bilayer vesicle aggregates capable of crossing pores smaller than the aggregate diameter without experiencing more than 50% fragmentation.

6. Composition for use in diminishing inflammation and/or pain in a mammal comprising adaptable vesicular aggregates, wherein said aggregates comprise at least one amphipat,
wherein the at least one amphipat contains at least one fluid hydrophobic segment with nC carbon atoms, and
wherein the hydrophobic segment of the amphipat is directly or indirectly attached to at least one hydrophilic headgroup having about 5nC/24 to about 8.5nC/24 polarity units per hydrophobic segment, and optionally
wherein the at least one amphipat can be supplemented with one or more additional amphipats, each having one or more polar headgroup, wherein the concentration of said additional amphipats, if any, is selected such that the average total of all polarity units on all amphipats is about 8.5 nC/24 polarity units per hydrophobic segment.

7. The composition for the use of claim 1, wherein said aggregates comprise:
one or more amphipats that can be dispersed into bilayer vesicles in a polar fluid, wherein said dispersion occurs at least two-fold faster when exposed to an external stress, such as a vigorous mechanical agitation, compared to a similarly concentrated and buffered reference aggregates composition made of at least 90% pure soybean phosphatidylcholine,

8. The composition for the use according to claims 1,
wherein the composition further comprises non-ionic and/or zwitterionic and/or amphoteric amphipats, and
wherein the headgroup of a non-ionic amphipat is comprised of one or more hydrophilic segments attached to one or more fluid hydrophobic segments that together have a total of between at least 8 up to about 24 carbon atoms, and
wherein the total number of side-chains and/or side-groups and/or double bonds, if any, in the hydrophobic segments is between 1 and 3.

9. The composition for the use of claim 8,
wherein the at least one hydrophilic segment is a pharmacologically acceptable polar group or a polymer thereof, selectable from:
a lower, linear or branched, alkyl chain alcohol that is hydroxylated on at least 50% of its carbons or
a sugar or an oligomer or lactone of said sugar, or
an amine oxide or its alkyl or dialkyl derivative, or
an amino or imino acid, or
a betaine or sulphobetaine, or
an aminoalkane sulphonic or sulphinic acid, an 1-amino-1-sulphosulphanylalkane, a dimethylammonio-1-alkanesulphonic, dimethylammonio-1-phosphonic, or dimethylammonio-1-acetic acid, a phospho-S,S-dimethyl mercapto short chain alkanol, or a secondary or ternary sulpho- or sulphono-short chain (poly)alkanolamine.

10. Composition for use in diminishing inflammation and/or pain in a mammal, comprising adaptable vesicular aggregates, wherein said aggregates comprise at least one amphipat, and:
the at least one amphipat has n fluid hydrophobic segments with a total of nC carbon atoms that are attached directly or indirectly to a zwitterionic or anionic headgroup of the at least one amphipat, said headgroup comprising an anionic phospho-, sulpho-, or arseno-moiety and optionally a cationic moiety, and
wherein the cationic moiety in the headgroup, if any, is a ternary or quaternary amine attached through a linker to the anionic moiety and
wherein the anionic moiety in the head group can be alkylated, coupled to a lower alkyl alcohol, an amino acid, a sugar, or to an oligomer thereof, and
wherein the at least one amphipat is optionally supplemented with a further amphipat, which is more polar if n=2 and less polar if n=1, and
wherein the overall polarity units count is between around 5nC/24 and about 8.5nC/24 per hydrophobic segment and the molar ratio of the first and the optional second amphipat, if more polar, exceeds 1/1.25.

11. The composition for the use of claim 10,
wherein the first amphipat comprises two hydrophobic segments having a total of at least 20 carbon atoms and the zwitterionic headgroup is a phosphoalkanol-dimethylamine or sulphoalkanol-dimethylamine or a phosphoalkanol-trimethylamine or sulphoalkanol-trimethylamine, and
wherein the second amphipat is a surfactant with an area per chain exceeding the area per chain of the first amphipat, and
wherein the relative concentration of the first amphipat is selected such that the overall average area per chain in the mixed aggregate is 0.43±0.05 nm².

12. The composition for the use of claim 1, wherein the total dry mass of aggregate forming components is between about 1 wt.-% and 40 wt.-%.

13. The composition for the use of claim 1, wherein the composition pH is between 3 and 9.5, and
wherein for uncharged aggregates comprising ester-bonded molecules, the pH is between about 5 to about 8, and
wherein for the positively charged aggregates containing amphipats having hydrolysable headgroup-fatty-chain bonds, the pH is between about 3 and about 6, and
wherein for the negatively charged aggregates comprised of amphipats with hydrolysable headgroup-fatty-chain bonds the pH is between around 7 and around 9.5.

14. The composition for the use of claim 1, wherein the aggregate composition has an average diameter between around 20 nm and around 1000 nm.

15. The composition for the use of claim 1, wherein the aggregate composition is packaged into a multiple-dosing container.

16. The composition for the use of claim 1, wherein the aggregate composition is administered on mammalian skin without an occlusive dressing in a quantity yielding a total amphipat mass per unit area of between 0.01 mg cm⁻² and 2.5 mg cm⁻², and more specifically 0.15±0.075 mg cm⁻² for superficial tissue treatment and about 1.5±0.75 mg cm⁻² for deep tissue treatment.

17. A composition for the use of claim 16, wherein the administration is repeated from 1 to 6 times daily

18. A composition for the use of claim 17, wherein an overall treatment duration is between 1 and 3 weeks for acute indications, and between 4 and 156 weeks for chronic indications.

19. The composition for the use of claim 1, wherein at least one additive is added to the aggregate composition and selected to act as a buffer and/or an antioxidant and/or a microbicide and/or a humectant and/or a fragrance and/or a co-solvent.

20. A composition for the use of claim 19, wherein the at least one additive increases the average area per hydrophobic chain of the aggregate forming amphipats to thereby improve the aggregate adaptability.

21. (canceled)

22. (canceled)
